(19) United States
(12) Patent Application Publication
(10) Pub. No.: US 2018/0221407 A1
(43) Pub. Date: Aug. 9, 2018

(54) OPHTHALMIC COMPOSITIONS FOR THERAPEUTIC AND PROPHYLACTIC USES

(71) Applicant: THE ADMINISTRATORS OF THE TULANE EDUCATIONAL FUND, New Orleans, LA (US)

(72) Inventors: Chad ROY, Madisonville, LA (US); Rebecca METZINGER, New Orleans, LA (US)

(21) Appl. No.: 15/887,870

(22) Filed: Feb. 2, 2018

Related U.S. Application Data
(60) Provisional application No. 62/454,585, filed on Feb. 3, 2017.
(60) Provisional application No. 62/583,350, filed on Nov. 8, 2017.

(51) Int. Cl. A61K 33/20; A61P 27/02; A61P 29/00; A61K 9/08; A61K 31/14; A61P 27/04; A61K 9/00; A61P 31/04; A61P 31/12; A61P 31/10; A61K 47/02; A61P 31/18

(52) U.S. Cl. CPC A61K 33/20; A61P 27/02; A61P 29/00; A61K 9/08; A61K 31/14; A61P 31/18; A61K 9/0048; A61P 31/04; A61P 31/12; A61P 31/10; A61K 47/02; A61P 27/04

(57) ABSTRACT

Disclosed herein are topical ophthalmic compositions comprising ammonium chloride, a quaternary ammonium salt, and stabilized chlorine dioxide. Also disclosed herein are methods of treating or preventing active infections of the tissues comprising the eye and surrounding parenchyma from bacterial, fungal, mycobacterial, mycoplasmal, viral, and protozoan amoeba microorganisms.

### CROSS-REFERENCE TO RELATED APPLICATIONS

This patent application claims the benefit of U.S. Provisional Patent Application No. 62/454,585, filed Feb. 3, 2017; and U.S. Provisional Patent Application No. 62/583,350, filed Nov. 8, 2017; each of which is incorporated herein by reference in their entirety.

### BACKGROUND

The present invention relates to pharmaceutical compositions and methods for treating or preventing eye conditions selected from keratitis, conjunctivitis, and blepharitis. The pharmaceutical compositions described herein comprise (a) a chlorite salt; (b) a quaternary ammonium salt; and (c) ammonium chloride.

### BRIEF SUMMARY OF THE INVENTION

Disclosed herein is a method of treating or preventing an eye condition in a subject in need thereof, the method comprising administering to the subject in need thereof a pharmaceutical composition comprising:
(a) a chlorite salt;
(b) a quaternary ammonium salt; and
(c) ammonium chloride.

In some embodiments of a method of treating or preventing an eye condition, the eye condition is an inflammation. In some embodiments of a method of treating or preventing an eye condition, the inflammation is inflammation of the eyelid. In some embodiments of a method of treating or preventing an eye condition, the inflammation of the eyelid is blepharitis. In some embodiments of a method of treating or preventing an eye condition, the blepharitis is associated with an internal hordeolum. In some embodiments of a method of treating or preventing an eye condition, the blepharitis is associated with an external hordeolum (stye). In some embodiments of a method of treating or preventing an eye condition, the blepharitis is associated with a chalazion. In some embodiments of a method of treating or preventing an eye condition, the blepharitis is posterior blepharitis. In some embodiments of a method of treating or preventing an eye condition, the blepharitis is anterior blepharitis. In some embodiments of a method of treating or preventing an eye condition, the inflammation of the eyelid is meibomianitis. In some embodiments of a method of treating or preventing an eye condition, the inflammation is inflammation of the interior of the eye. In some embodiments of a method of treating or preventing an eye condition, the inflammation is endophthalmitis. In some embodiments of a method of treating or preventing an eye condition, the inflammation is inflammation of the conjuctiva. In some embodiments of a method of treating or preventing an eye condition, the inflammation is conjunctivitis. In some embodiments of a method of treating or preventing an eye condition, the inflammation is inflammation of the cornea. In some embodiments of a method of treating or preventing an eye condition, the inflammation is keratitis. In some embodiments of a method of treating or preventing an eye condition, the inflammation is caused by a bacterial infection. In some embodiments of a method of treating or preventing an eye condition, the inflammation is caused by a viral infection. In some embodiments of a method of treating or preventing an eye condition, the inflammation is caused by a fungal infection. In some embodiments of a method of treating or preventing an eye condition, the inflammation is caused by a parasitic infection. In some embodiments of a method of treating or preventing an eye condition, the inflammation is caused by an amoebic infection.

Also disclosed herein is a method of treating or preventing an infection in a subject in need thereof, the method comprising administering to the subject in need thereof a pharmaceutical composition comprising:
(a) a chlorite salt;
(b) a quaternary ammonium salt; and
(c) ammonium chloride.

In some embodiments of a method of treating or preventing an infection, the infection is a bacterial infection. In some embodiments of a method of treating or preventing an infection, the infection is a fungal infection. In some embodiments of a method of treating or preventing an infection, the infection is a mycobacterial infection. In some embodiments of a method of treating or preventing an infection, the infection is a mycoplasma infection. In some embodiments of a method of treating or preventing an infection, the infection is a viral infection. In some embodiments of a method of treating or preventing an infection, the infection is caused by a protozoan microorganism. In some embodiments of a method of treating or preventing an infection, the infection is an eye infection. In some embodiments of a method of treating or preventing an infection, the infection is associated with conjunctivitis, keratitis, blepharitis, meibomianitis, or endophthalmitis. In some embodiments of a method of treating or preventing an infection, the infection is associated with acanthamoebic keratitis.

In some embodiments of a method of disclosed herein, the chlorite salt is present in an amount ranging from about 0.0001% to about 1% (w/w). In some embodiments of a method of disclosed herein, the chlorite salt is present in an amount ranging from about 0.0001% to about 0.1% (w/w). In some embodiments of a method of disclosed herein, the chlorite salt is present in an amount ranging from about 0.0001% to about 0.01% (w/w). In some embodiments of a method of disclosed herein, the chlorite salt is present in an amount ranging from about 0.0001% to about 0.005% (w/w). In some embodiments of a method of disclosed herein, the chlorite salt is present in an amount ranging from about 0.0001% to about 0.001% (w/w). In some embodiments of a method of disclosed herein, the chlorite salt is present in an amount ranging from about 0.0001% to about 0.0005% (w/w). In some embodiments of a method of disclosed herein, the chlorite salt is present in an amount of about 0.0003% (w/w). In some embodiments of a method of disclosed herein, the chlorite salt is present in an amount of about 0.0006% (w/w). In some embodiments of a method of disclosed herein, the chlorite salt is present in an amount of about 0.0009% (w/w). In some embodiments of a method of disclosed herein, the chlorite salt is an alkali metal chlorite salt. In some embodiments of a method of disclosed herein, the alkali metal chlorite salt is sodium chlorite. In some embodiments of a method of disclosed herein, the sodium chlorite is provided as a stabilized chlorine dioxide solution. In some embodiments of a method of disclosed herein, the stabilized chlorine dioxide is present in an amount ranging from about 0.005% to about 1% (w/w). In some embodiments of a method of disclosed herein, the stabilized chlorine dioxide is present in an amount ranging from about 0.005% to about 0.1% (w/w). In some embodiments of a method of disclosed herein, the stabilized chlorine dioxide is present in an amount ranging from about 0.01% to about 0.1% (w/w). In some embodiments of a method of disclosed herein, the stabilized chlorine dioxide is present in an amount ranging from about 0.005% to about 0.01% (w/w). In some embodiments of a method of disclosed herein, the stabilized chlorine dioxide is present in an amount ranging from about 0.009% to about 0.011% (w/w). In some embodiments of a method of disclosed herein, the stabilized chlorine dioxide is present in an amount of about 0.01% (w/w). In some embodiments of a method of disclosed herein, the stabilized chlorine dioxide is present in an amount ranging from about 0.01% to about 0.05% (w/w). In some embodiments of a method of disclosed herein, the stabilized chlorine dioxide is present in an amount of about 0.02% (w/w). In some embodiments of a method of disclosed herein, the stabilized chlorine dioxide is present in an amount of about 0.03% (w/w). In some embodiments of a method of disclosed herein, the stabilized chlorine dioxide solution comprises chlorine dioxide. In some embodiments of a method of disclosed herein, the quaternary ammonium salt is present in an amount ranging from about 0.0001% to about 0.5% (w/w). In some embodiments of a method of disclosed herein, the quaternary ammonium salt is present in an amount ranging from about 0.0001% to about 0.1% (w/w). In some embodiments of a method of disclosed herein, the quaternary ammonium salt is present in an amount ranging from about 0.0001% to about 0.01% (w/w). In some embodiments of a method of disclosed herein, the quaternary ammonium salt is present in an amount ranging from about 0.0001% to about 0.002% (w/w). In some embodiments of a method of disclosed herein, the quaternary ammonium salt is present in an amount ranging from about 0.0005% to about 0.002% (w/w). In some embodiments of a method of disclosed herein, the quaternary ammonium salt is present in an amount ranging from about 0.0005% to about 0.0012% (w/w). In some embodiments of a method of disclosed herein, the quaternary ammonium salt is present in an amount ranging from about 0.0005% to about 0.005% (w/w). In some embodiments of a method of disclosed herein, the quaternary ammonium salt is present in an amount ranging from about 0.001% to about 0.005% (w/w). In some embodiments of a method of disclosed herein, the quaternary ammonium salt is present in an amount of about 0.001% (w/w). In some embodiments of a method of disclosed herein, the quaternary ammonium salt is present in an amount of about 0.003% (w/w). In some embodiments of a method of disclosed herein, the quaternary ammonium salt comprises C12 or C14 alkyl chain. In some embodiments of a method of disclosed herein, the quaternary ammonium salt is not benzalkonium chloride. In some embodiments of a method of disclosed herein, the quaternary ammonium salt is a polymeric quaternary ammonium salt. In some embodiments of a method of disclosed herein, the quaternary ammonium salt is C12-C14-alkyl(ethylbenzyl)dimethylammonium chloride. In some embodiments of a method of disclosed herein, the ammonium chloride is present in an amount ranging from about 0.001% to about 2% (w/w). In some embodiments of a method of disclosed herein, the ammonium chloride is present in an amount ranging from about 0.001% to about 1% (w/w). In some embodiments of a method of disclosed herein, the ammonium chloride is present in an amount ranging from about 0.01% to about 1% (w/w). In some embodiments of a method of disclosed herein, the ammonium chloride is present in an amount ranging from about 0.1% to about 1% (w/w). In some embodiments of a method of disclosed herein, the ammonium chloride is present in an amount ranging from about 0.01% to about 0.3% (w/w). In some embodiments of a method of disclosed herein, the ammonium chloride is present in an amount ranging from about 0.1% to about 0.3% (w/w). In some embodiments of a method of disclosed herein, the ammonium chloride is present in an amount of about 0.2% (w/w). In some embodiments of a method of disclosed herein, the ammonium chloride is present in an amount ranging from about 0.001% to about 0.05% (w/w). In some embodiments of a method of disclosed herein, the ammonium chloride is present in an amount ranging from about 0.01% to about 0.05% (w/w). In some embodiments of a method of disclosed herein, the ammonium chloride is present in an amount of about 0.025% (w/w). In some embodiments of a method of disclosed herein, the pharmaceutical composition further comprises a pharmaceutically acceptable excipient. In some embodiments of a method of disclosed herein, the pharmaceutically acceptable excipient is selected from a surfactant, a chelating agent, a tonicity agent, a viscosity agent, a non-aqueous solvent, a buffer, a pH adjusting agent, an antioxidant, and any combinations thereof. In some embodiments of a method of disclosed herein, the pharmaceutically acceptable excipient is selected from a surfactant, a chelating agent, a tonicity agent, a buffer, and any combinations thereof. In some embodiments of a method of disclosed herein, the pharmaceutical composition further comprises a surfactant. In some embodiments of a method of disclosed herein, the surfactant is selected from sodium lauryl sulfate, docusate sodium, polyoxyalkyl ethers, polyoxylalkyl phenyl ethers, polyoxyl castor oils, polyoxyl hydrogenated castor oils, polyoxyl 40 stearates, polyoxy sorbitan esters, sorbitan esters, polysorbates, sorbitan monolaureates, poloxamines, poloxamers, and any combinations thereof. In some embodiments of a method of disclosed herein, the surfactant is Tetronic® 908. In some embodiments of a method of disclosed herein, the Tetronic® 908 is present in an amount ranging from about 0.1% and about 1% (w/w). In some embodiments of a method of disclosed herein, the Tetronic® 908 is present in an amount of about 0.25% (w/w). In some embodiments of a method of disclosed herein, the Tetronic® 908 is present in an amount ranging from about 0.01% and about 0.5% (w/w). In some embodiments of a method of disclosed herein, the Tetronic® 908 is present in an amount of about 0.02% (w/w). In some embodiments of a method of disclosed herein, the pharmaceutical composition further comprises a tonicity agent. In some embodiments of a method of disclosed herein, the tonicity agent is selected from sodium chloride, potassium chloride, mannitol, dextrose, glycerin, propylene glycol, and any combinations thereof. In some embodiments of a method of disclosed herein, the tonicity agent is sodium chloride. In some embodiments of a method of disclosed herein, the sodium chloride is present in an amount ranging from about 0.05% and about 1% (w/w). In some embodiments of a method of disclosed herein, the sodium chloride is present in an amount of about 0.1% (w/w). In some embodiments of a method of disclosed herein, the tonicity agent is potassium chloride. In some embodiments of a method of disclosed herein, the potassium chloride is present in an amount ranging from about 0.05% and about 1% (w/w). In some embodiments of a method of disclosed herein, the potassium chloride is present in an amount of about 0.2% (w/w). In some embodiments of a method of disclosed herein, the tonicity agent is propylene glycol. In some embodiments of a method of disclosed herein, the propylene glycol is present in an amount ranging from about 0.05% and about 1% (w/w). In some embodiments of a method of disclosed herein, the propylene glycol is present in an amount of about 0.75% (w/w). In some embodiments of a method of disclosed herein, the propylene glycol is present in an amount ranging from about 0.001% and about 0.1% (w/w). In some embodiments of a method of disclosed herein, the propylene glycol is present in an amount of about 0.0075% (w/w). In some embodiments of a method of disclosed herein, the pharmaceutical composition further comprises a chelating agent. In some embodiments of a method of disclosed herein, the chelating agent is selected from EDTA, disodium edetate, and any combinations thereof. In some embodiments of a method of disclosed herein, the chelating agent is present in an amount ranging from about 0.01% and about 1% (w/w). In some embodiments of a method of disclosed herein, the chelating agent is present in an amount of about 0.05% (w/w). In some embodiments of a method of disclosed herein, the pharmaceutical composition further comprises a buffer to maintain the pH between about 7 and about 8. In some embodiments of a method of disclosed herein, the pH is about 7.4. In some embodiments of a method of disclosed herein, the pharmaceutical composition is in the form of a solution, an emulsion, a solid dispersion, a colloidal dispersion, a suspension, a gel, an ointment, a cream, a lyophilized powder, a nanoparticle formulation, or any combinations thereof. In some embodiments of a method of disclosed herein, the pharmaceutical composition is applied with a pad, cloth, wipe, sponge, brush, or cotton tipped applicator. In some embodiments of a method of disclosed herein, the pharmaceutical composition is in the form of a surgical wash or irrigation solution. In some embodiments of a method of disclosed herein, the surgical wash or irrigation solution is applied to the eye of the subject in need thereof. In some embodiments of a method of disclosed herein, the surgical wash or irrigation solution is applied to the eye of the subject in need thereof prior to a surgery. In some embodiments of a method of disclosed herein, the surgical wash or irrigation solution is applied to the eye of the subject in need thereof after a surgery. In some embodiments of a method of disclosed herein, the surgery is laser eye surgery, cataract surgery, glaucoma surgery, refractive surgery, corneal surgery, vitreo-retinal surgery, eye muscle surgery, oculoplastic surgery, surgery involving the lacrimal apparatus, or eye removal. In some embodiments of a method of disclosed herein, the pharmaceutical composition is applied by topical administration. In some embodiments of a method of disclosed herein, the infection or eye condition is blepharitis. In some embodiments of a method of disclosed herein, the infection or eye condition is meibomianitis. In some embodiments of a method of disclosed herein, the infection or eye condition is endophthalmitis. In some embodiments of a method of disclosed herein, the infection or eye condition is conjunctivitis. In some embodiments of a method of disclosed herein, the infection or eye condition is keratitis.

Also disclosed herein is a method of treating or preventing an eye condition in a subject in need thereof, the method comprising administering to the subject in need thereof a pharmaceutical composition comprising:
(a) a chlorite salt;
(b) a quaternary ammonium salt; and
(c) ammonium chloride.

In some embodiments of a method of treating or preventing an eye condition, the eye condition is an inflammation. In some embodiments of a method of treating or preventing an eye condition, the inflammation is conjunctivitis, keratitis, blepharitis, meibomianitis, endophthalmitis, or any combination thereof. In some embodiments of a method of treating or preventing an eye condition, the inflammation is conjunctivitis. In some embodiments of a method of treating or preventing an eye condition, the inflammation is acanthamoebic keratitis. In some embodiments of a method of treating or preventing an eye condition, the inflammation is caused by a bacterial infection. In some embodiments of a method of treating or preventing an eye condition, the inflammation is caused by a viral infection. In some embodiments of a method of treating or preventing an eye condition, the inflammation is caused by a fungal infection. In some embodiments of a method of treating or preventing an eye condition, the chlorite salt is present in an amount ranging from about 0.0001% to about 1% (w/w). In some embodiments of a method of treating or preventing an eye condition, the chlorite salt is an alkali metal chlorite salt. In some embodiments of a method of treating or preventing an eye condition, the alkali metal chlorite salt is sodium chlorite. In some embodiments of a method of treating or preventing an eye condition, the sodium chlorite is provided as a stabilized chlorine dioxide solution. In some embodiments of a method of treating or preventing an eye condition, the stabilized chlorine dioxide is present in an amount ranging from about 0.005% to about 1% (w/w). In some embodiments of a method of treating or preventing an eye condition, the quaternary ammonium salt is present in an amount ranging from about 0.0001% to about 0.5% (w/w). In some embodiments of a method of treating or preventing an eye condition, the quaternary ammonium salt comprises C12 or C14 alkyl chain. In some embodiments of a method of treating or preventing an eye condition, the quaternary ammonium salt is not benzalkonium chloride. In some embodiments of a method of treating or preventing an eye condition, the quaternary ammonium salt is a polymeric quaternary ammonium salt. In some embodiments of a method of treating or preventing an eye condition, the quaternary ammonium salt is C12-C14-alkyl(ethylbenzyl)dimethylammonium chloride. In some embodiments of a method of treating or preventing an eye condition, the ammonium chloride is present in an amount ranging from about 0.001% to about 2% (w/w). In some embodiments of a method of treating or preventing an eye condition, the pharmaceutical composition further comprises a pharmaceutically acceptable excipient. In some embodiments of a method of treating or preventing an eye condition, the pharmaceutically acceptable excipient is selected from a surfactant, a chelating agent, a tonicity agent, a viscosity agent, a non-aqueous solvent, a buffer, a pH adjusting agent, an antioxidant, and any combinations thereof. In some embodiments of a method of treating or preventing an eye condition, the pharmaceutical composition further comprises a buffer to maintain the pH between about 7 and about 8. In some embodiments of a method of treating or preventing an eye condition, the pH is about 7.4. In some embodiments of a method of treating or preventing an eye condition, the pharmaceutical composition is in the form of a solution, an emulsion, a solid dispersion, a colloidal dispersion, a suspension, a gel, an ointment, a cream, a lyophilized powder, a nanoparticle formulation, or any combinations thereof. In some embodiments of a method of treating or preventing an eye condition, the pharmaceutical composition is applied with a pad, cloth, wipe, sponge, brush, or cotton tipped applicator. In some embodiments of a method of treating or preventing an eye condition, the pharmaceutical composition is in the form of a surgical wash or irrigation solution. In some embodiments of a method of treating or preventing an eye condition, the surgical wash or irrigation solution is applied to the eye of the subject in need thereof. In some embodiments of a method of treating or preventing an eye condition, the surgical wash or irrigation solution is applied to the eye of the subject in need thereof prior to a surgery. In some embodiments of a method of treating or preventing an eye condition, the surgical wash or irrigation solution is applied to the eye of the subject in need thereof after a surgery. In some embodiments of a method of treating or preventing an eye condition, the pharmaceutical composition is applied by topical administration.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the description of specific embodiments presented herein.

FIG. 1 shows cytotoxicity rankings of the two presently-disclosed multipurpose disinfection solutions (SOL01 and SOL02) and three commercially available multipurpose disinfection solutions: BioTrue (SOL05)<Opti-Free (SOL03)≤ReNu (SOL04)≤SOL01<SOL02.

FIG. 2 shows the cytotoxic activity of the two presently-disclosed multipurpose disinfection solutions (SOL01 and SOL02).

FIG. 3 shows a comparison of the two presently-disclosed multipurpose disinfection solutions (SOL01 and SOL02) and three commercially available multipurpose disinfection solutions (BioTrue (SOL05), Opti-Free (SOL03), and ReNu (SOL04)).

FIG. 4 shows the results of one hour of contact time with the two presently-disclosed multipurpose disinfection solutions (SOL01 and SOL02) and three commercially available multipurpose disinfection solutions (BioTrue (SOL05), Opti-Free (SOL03), and ReNu (SOL04)) for P. aeruginosa (ATCC 9027).

FIG. 5 shows the results of one hour of contact time with the two presently-disclosed multipurpose disinfection solutions (SOL01 and SOL02) and three commercially available multipurpose disinfection solutions (BioTrue (SOL05), Opti-Free (SOL03), and ReNu (SOL04)) for S. aureus (ATCC 6538).

FIG. 6 shows the results of two hours of contact time with the two presently-disclosed multipurpose disinfection solutions (SOL01 and SOL02) and two commercially available multipurpose disinfection solutions (BioTrue (SOL05), Opti-Free (SOL03), and ReNu (SOL04)) for P. aeruginosa (ATCC 9027).

FIG. 7 shows the results of four hours of contact time the two presently-disclosed multipurpose disinfection solutions (SOL01 and SOL02) and three commercially available multipurpose disinfection solutions(BioTrue (SOL05), Opti-Free (SOL03), and ReNu (SOL04)) for P. aeruginosa (ATCC 9027).

FIG. 8 shows the results of four hours of contact time with the two presently-disclosed multipurpose disinfection solutions (SOL01 and SOL02) and three commercially available multipurpose disinfection solutions (BioTrue (SOL05), Opti-Free (SOL03), and ReNu (SOL04)) for S. aureus (ATCC 6538).

FIG. 9 shows the results of one hour and four hours of contact time with two presently-disclosed multipurpose disinfection solutions (SOL01 and SOL02) and three commercially available multipurpose disinfection solutions (BioTrue (SOL05), Opti-Free (SOL03), and ReNu (SOL04)) for S. marcescens.

FIG. 10 shows the results of one hour and four hours of contact time with two presently-disclosed multipurpose disinfection solutions (SOL01 and SOL02) and three commercially available multipurpose disinfection solutions (BioTrue (SOL05), Opti-Free (SOL03), and ReNu (SOL04)) for C. albicans.

FIG. 11 shows the results of one hour and four hours of contact time with two presently-disclosed multipurpose disinfection solutions (SOL01 and SOL02) and three commercially available multipurpose disinfection solutions (BioTrue (SOL05), Opti-Free (SOL03), and ReNu (SOL04)) for F. solani.

FIG. 12A shows the results of 96 h incubation of STR-325 and two commercially available multipurpose disinfection solutions (ReNu and Biotrue) for A. castellanii.

FIG. 12B shows the results of lh incubation of STR-325 and two commercially available multipurpose disinfection solution (ReNu and Biotrue) for A. castellanii.

FIG. 13 shows the results of STR-325, chlorhexidine, and povidone-iodine for A. castellanii.

FIG. 14 shows the %cyst kills results of STR-325 and two commercially available multipurpose disinfection solution (ReNu and Biotrue) for A. castellanii.

FIG. 15 shows the results of 30 min incubation of Formulation A and two commercially available multipurpose disinfection solution (ReNu and Biotrue) for A. castellanii. Microscopic examination was done using Trypan Blue. Dark inclusions denote death of the Acanthamoeba.

### DETAILED DESCRIPTION

Detailed descriptions of one or more embodiments are provided herein. It is to be understood, however, that the present disclosure is embodied in various forms. Therefore, specific details disclosed herein are not to be interpreted as limiting, but rather as a basis for the claims and as a representative basis for teaching one skilled in the art to employ the present disclosure in any appropriate manner.

Wherever the phrase “for example,” “such as,” “including,” and the like are used herein, the phrase “and without limitation” is understood to follow unless explicitly stated otherwise Similarly “an example,” “exemplary,” and the like are understood to be non-limiting.

The term “substantially” allows for deviations from the descriptor that don't negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The term “about” is used to indicate that a value includes the standard level of error for the device or method being employed to determine the value. In some embodiments, the level of error is 10%. In some embodiments, the level of error is 9%. In some embodiments, the level of error is 8%. In some embodiments, the level of error is 7%. In some embodiments, the level of error is 6%. In some embodiments, the level of error is 5%.

The terms “comprising,” “including,” “having,” “involving” (and similarly “comprises,” “includes,” “has,” and “involves”), and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

“Accelerated conditions” as used herein include temperature and/or relative humidity (RH) that are at or above ambient levels (e.g. 25±5° C.; 55±10% RH). In some instances, an accelerated condition is at about 25° C., about 30° C., about 35° C., about 40° C., about 45° C., about 50° C., about 55° C. or about 60° C. In other instances, an accelerated condition is above 55% RH, about 65% RH, about 70% RH, about 75% RH, or about 80% RH. In further instances, an accelerated condition is about 40° C. or 60° C. at ambient humidity. In yet further instances, an accelerated condition is about 40° C. at 75±5% RH humidity.

“Refrigerated condition” as used herein refer to 5±3° C. In some embodiments, refrigerated condition is about 2° C., about 2.1° C., about 2.2° C., about 2.3° C., about 2.4° C., about 2.5° C., about 2.6° C., about 2.7° C., about 2.8° C., about 2.9° C., about 3° C., about 3.1° C., about 3.2° C., about 3.3° C., about 3.4° C., about 3.5° C., about 3.6° C., about 3.7° C., about 3.8° C., about 3.9° C., about 4° C., about 4.1° C., about 4.2° C., about 4.3° C., about 4.4° C., about 4.5° C., about 4.6° C., about 4.7° C., about 4.8° C., about 4.9° C., about 5° C., about 5.1° C., about 5.2° C., about 5.3° C., about 5.4° C., about 5.5° C., about 5.6° C., about 5.7° C., about 5.8° C., about 5.9° C., about 6° C., about 6.1° C., about 6.2° C., about 6.3° C., about 6.4° C., about 6.5° C., about 6.6° C., about 6.7° C., about 6.8° C., about 6.9° C., about 7° C., about 7.1° C., about 7.2° C., about 7.3° C., about 7.4° C., about 7.5° C., about 7.6° C., about 7.7° C., about 7.8° C., about 7.9° C., or about 8° C.

The present disclosure recognizes that chemotherapeutic agents for the treatment of infectious conjunctivitis are available. The mechanism of action of each therapeutic target depends on the particular type of infectious organism responsible for the ongoing infection. These include antibiotics for treatment of bacterial infection, antifungals for treatment of fungal infections, and antivirals for the treatment of viral infections. It is recognized that there is no commercially available treatment for an infection that may be caused by microorganism classified in a different Kingdom. The present disclosure recognizes that biocidal agents, such as povidone-iodine and biguanide organic salts, are used for the treatment of ophthalmic conjunctivitis. It is however recognized that these biocidal agents possess a singular, weak mechanism of action for microbial killing in general and are both associated with overt cellular and tissue toxicity, and clinical pain upon administration. In addition, current biocides for this use possess minimal efficacy against recalcitrant organisms such as Gram-negative bacteria (e.g., Pseudomonas aeruginosa) or multiform infectious protozoa (e.g., Acanthamoeba spp.).

The present disclosure recognizes that diagnosis leading to the identification of the microbial source of infectious conjunctivitis is difficult clinically. In lieu of accurate and precise diagnosis from laboratory-based clinical microbiological identification, the health practitioner usually provides recommended therapeutic relief based upon a combination of clinical experience and a clinical examination of the infected eye. In the majority of cases topically applied antibiotics are prescribed as a default therapy with the presumptive anticipation that the causative agent of the conjunctivitis is bacterial. This approach and therapeutic choice provides minimal benefit to individuals suffering from infectious conjunctivitis from a source other than bacterial, as antibiotic therapies are not effective against fungal, viral, or amoebic infection.

In addition, clinical standard-of-care dictates that the currently commercially available biocidal agents are generally not prescribed as first-line treatment for unclassified infectious conjunctivitis due to the low efficacy and toxicity associated with use. These agents are used in the event that either an unusually recalcitrant organism has been identified as the source of the infection and first-line targeted therapeutics have failed to resolve the infection, or the causative agent of the infection is an infectious protozoa. When prescribed, biocidal therapy is considered the “last resort” for clinical resolution of infectious conjunctivitis. It is recognized that therapeutic use and prescriptive regiment associated with administration of either povidone-iodine or biguanide organic salts is the source of significant ocular pain upon administration and is represented by generalized inflammation throughout the treated area stemming from the toxicity associated with either agent's use.

The present disclosure recognizes that there is no currently marketed broad-spectrum ophthalmic chemotherapeutic agent that is effective in killing and/or deactivating all infectious causes of conjunctivitis, keratitis, or blepharitis that are defined within the scope of the aforementioned Kingdom classifications. Nor is there a solution available that can be used preoperatively or in other situations requiring sterile field that can be safely used as a prophylactic against microbial invasions and ensuing infections.

### Pharmaceutical Compositions

Disclosed herein are pharmaceutical compositions comprising (a) a chlorite salt; (b) a quaternary ammonium salt; and (c) ammonium chloride. In some embodiments, the pharmaceutical composition further comprises an aqueous carrier. In some embodiments, the pharmaceutical composition further comprises an aqueous carrier that is water. In some embodiments, the pharmaceutical composition further comprises a pharmaceutically acceptable excipient. In some embodiments, the pharmaceutically acceptable excipient is selected from a surfactant, a tonicity agent, a viscosity agent, a chelating agent, a non-aqueous solvent, a buffer, a pH adjusting agent, an antioxidant, and any combinations thereof. Exemplary pharmaceutically acceptable excipients of the disclosure include those found in Remington: The Science and Practice of Pharmacy, Twenty Second Ed. (London, UK: Pharmaceutical Press, 2013) incorporated herein by reference for such disclosure.

In some embodiments, the pharmaceutical composition comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride.

In some embodiments, the pharmaceutical composition comprises (a) about 0.0003% (w/w) of a chlorite salt; (b) about 0.001% (w/w) of a quaternary ammonium salt; and (c) about 0.2% (w/w) of ammonium chloride.

In some embodiments, the pharmaceutical composition comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride.

In some embodiments, the pharmaceutical composition comprises (a) about 0.0003% (w/w) of sodium chlorite; (b) about 0.001% (w/w) of a quaternary ammonium salt; and (c) about 0.2% (w/w) of ammonium chloride.

In some embodiments, the pharmaceutical composition comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride.

In some embodiments, the pharmaceutical composition comprises (a) about 0.01% (w/w) of stabilized chlorine dioxide; (b) about 0.001% (w/w) of a quaternary ammonium salt; and (c) about 0.2% (w/w) of ammonium chloride.

In some embodiments, the pharmaceutical composition comprises (a) about 0.02% (w/w) of stabilized chlorine dioxide; (b) about 0.001% (w/w) of a quaternary ammonium salt; and (c) about 0.025% (w/w) of ammonium chloride.

In some embodiments, the pharmaceutical composition comprises (a) about 0.03% (w/w) of stabilized chlorine dioxide; (b) about 0.003% (w/w) of a quaternary ammonium salt; and (c) about 0.025% (w/w) of ammonium chloride.

### Chlorite Salt

In some aspects, the pharmaceutical composition comprises a chlorite salt. In some embodiments, the chlorite salt is an alkaline earth metal chlorite salt. In some embodiments, the chlorite salt is an alkali metal chlorite salt. In some embodiments, the chlorite salt is sodium chlorite. In some embodiments, the chlorite salt, e.g., sodium chlorite, is present in the pharmaceutical composition in an amount ranging from about 0.0001% to about 1% (w/w). In some embodiments, the chlorite salt, e.g., sodium chlorite, is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% (w/w). In some embodiments, the chlorite salt, e.g., sodium chlorite, is present in the pharmaceutical composition in an amount ranging from about 0.0001% to about 0.1% (w/w). In some embodiments, the chlorite salt, e.g., sodium chlorite, is present in the pharmaceutical composition in an amount ranging from about 0.0001% to about 0.01% (w/w). In some embodiments, the chlorite salt, e.g., sodium chlorite, is present in the pharmaceutical composition in an amount ranging from about 0.0001% to about 0.005% (w/w). In some embodiments, the chlorite salt, e.g., sodium chlorite, is present in the pharmaceutical composition in an amount ranging from about 0.0001% to about 0.001% (w/w). In some embodiments, the chlorite salt, e.g., sodium chlorite, is present in the pharmaceutical composition in an amount ranging from about 0.0001% to about 0.001% (w/w). In some embodiments, the chlorite salt, e.g., sodium chlorite, is present in the pharmaceutical composition in an amount ranging from about 0.0001% to about 0.0005% (w/w). In some embodiments, the chlorite salt, e.g., sodium chlorite, is present in the pharmaceutical composition in an amount of about 0.0003% (w/w). In some embodiments, the chlorite salt, e.g., sodium chlorite, is present in the pharmaceutical composition in an amount of about 0.0006% (w/w). In some embodiments, the chlorite salt, e.g., sodium chlorite, is present in the pharmaceutical composition in an amount of about 0.0009% (w/w).

In some embodiments, the chlorite salt, e.g., sodium chlorite, is present in the pharmaceutical composition in an amount ranging from about 0.0001% to about 10% (w/w). In some embodiments, the chlorite salt, e.g., sodium chlorite, is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w). In some embodiments, the chlorite salt, e.g., sodium chlorite, is present in the pharmaceutical composition in an amount ranging from about 0.0001% to about 5% (w/w). In some embodiments, the chlorite salt, e.g., sodium chlorite, is present in the pharmaceutical composition in an amount ranging from about 0.0001% to about 4% (w/w). In some embodiments, the chlorite salt, e.g., sodium chlorite, is present in the pharmaceutical composition in an amount ranging from about 0.0001% to about 3% (w/w). In some embodiments, the chlorite salt, e.g., sodium chlorite, is present in the pharmaceutical composition in an amount ranging from about 0.0001% to about 2% (w/w). In some embodiments, the chlorite salt, e.g., sodium chlorite, is present in the pharmaceutical composition in an amount ranging from about 0.0001% to about 1% (w/w). In some embodiments, the chlorite salt, e.g., sodium chlorite, is present in the pharmaceutical composition in an amount ranging from about 0.0001% to about 0.1% (w/w). In some embodiments, the chlorite salt, e.g., sodium chlorite, is present in the pharmaceutical composition in an amount ranging from about 0.0001% to about 0.01% (w/w). In some embodiments, the chlorite salt, e.g., sodium chlorite, is present in the pharmaceutical composition in an amount ranging from about 0.0001% to about 0.001% (w/w). In some embodiments, the chlorite salt, e.g., sodium chlorite, is present in the pharmaceutical composition in an amount ranging from about 0.001% to about 5% (w/w). In some embodiments, the chlorite salt, e.g., sodium chlorite, is present in the pharmaceutical composition in an amount ranging from about 0.001% to about 4% (w/w). In some embodiments, the chlorite salt, e.g., sodium chlorite, is present in the pharmaceutical composition in an amount ranging from about 0.001% to about 3% (w/w). In some embodiments, the chlorite salt, e.g., sodium chlorite, is present in the pharmaceutical composition in an amount ranging from about 0.001% to about 2% (w/w). In some embodiments, the chlorite salt, e.g., sodium chlorite, is present in the pharmaceutical composition in an amount ranging from about 0.001% to about 1% (w/w). In some embodiments, the chlorite salt, e.g., sodium chlorite, is present in the pharmaceutical composition in an amount ranging from about 0.001% to about 0.1% (w/w). In some embodiments, the chlorite salt, e.g., sodium chlorite, is present in the pharmaceutical composition in an amount ranging from about 0.001% to about 0.01% (w/w). In some embodiments, the chlorite salt, e.g., sodium chlorite, is present in the pharmaceutical composition in an amount ranging from about 0.01% to about 5% (w/w). In some embodiments, the chlorite salt, e.g., sodium chlorite, is present in the pharmaceutical composition in an amount ranging from about 0.01% to about 4% (w/w). In some embodiments, the chlorite salt, e.g., sodium chlorite, is present in the pharmaceutical composition in an amount ranging from about 0.01% to about 3% (w/w). In some embodiments, the chlorite salt, e.g., sodium chlorite, is present in the pharmaceutical composition in an amount ranging from about 0.01% to about 2% (w/w). In some embodiments, the chlorite salt, e.g., sodium chlorite, is present in the pharmaceutical composition in an amount ranging from about 0.01% to about 1% (w/w). In some embodiments, the chlorite salt, e.g., sodium chlorite, is present in the pharmaceutical composition in an amount ranging from about 0.01% to about 0.1% (w/w). In some embodiments, the chlorite salt, e.g., sodium chlorite, is present in the pharmaceutical composition in an amount ranging from about 0.001% to about 5% (w/w). In some embodiments, the chlorite salt, e.g., sodium chlorite, is present in the pharmaceutical composition in an amount ranging from about 0.01% to about 5% (w/w). In some embodiments, the chlorite salt, e.g., sodium chlorite, is present in the pharmaceutical composition in an amount ranging from about 0.1% to about 5% (w/w). In some embodiments, the chlorite salt, e.g., sodium chlorite, is present in the pharmaceutical composition in an amount ranging from about 1% to about 5% (w/w). In some embodiments, the chlorite salt, e.g., sodium chlorite, is present in the pharmaceutical composition in an amount ranging from about 2% to about 5% (w/w). In some embodiments, the chlorite salt, e.g., sodium chlorite, is present in the pharmaceutical composition in an amount ranging from about 3% to about 5% (w/w). In some embodiments, the chlorite salt, e.g., sodium chlorite, is present in the pharmaceutical composition in an amount ranging from about 4% to about 5% (w/w). In some embodiments, the chlorite salt, e.g., sodium chlorite, is present in the pharmaceutical composition in an amount ranging from about 0.001% to about 4% (w/w). In some embodiments, the chlorite salt, e.g., sodium chlorite, is present in the pharmaceutical composition in an amount ranging from about 0.01% to about 4% (w/w). In some embodiments, the chlorite salt, e.g., sodium chlorite, is present in the pharmaceutical composition in an amount ranging from about 0.1% to about 4% (w/w). In some embodiments, the chlorite salt, e.g., sodium chlorite, is present in the pharmaceutical composition in an amount ranging from about 1% to about 4% (w/w). In some embodiments, the chlorite salt, e.g., sodium chlorite, is present in the pharmaceutical composition in an amount ranging from about 2% to about 4% (w/w). In some embodiments, the chlorite salt, e.g., sodium chlorite, is present in the pharmaceutical composition in an amount ranging from about 3% to about 4% (w/w). In some embodiments, the chlorite salt, e.g., sodium chlorite, is present in the pharmaceutical composition in an amount ranging from about 0.001% to about 3% (w/w). In some embodiments, the chlorite salt, e.g., sodium chlorite, is present in the pharmaceutical composition in an amount ranging from about 0.01% to about 3% (w/w). In some embodiments, the chlorite salt, e.g., sodium chlorite, is present in the pharmaceutical composition in an amount ranging from about 0.1% to about 3% (w/w). In some embodiments, the chlorite salt, e.g., sodium chlorite, is present in the pharmaceutical composition in an amount ranging from about 1% to about 3% (w/w). In some embodiments, the chlorite salt, e.g., sodium chlorite, is present in the pharmaceutical composition in an amount ranging from about 2% to about 3% (w/w). In some embodiments, the chlorite salt, e.g., sodium chlorite, is present in the pharmaceutical composition in an amount ranging from about 0.001% to about 2% (w/w). In some embodiments, the chlorite salt, e.g., sodium chlorite, is present in the pharmaceutical composition in an amount ranging from about 0.01% to about 2% (w/w). In some embodiments, the chlorite salt, e.g., sodium chlorite, is present in the pharmaceutical composition in an amount ranging from about 0.1% to about 2% (w/w). In some embodiments, the chlorite salt, e.g., sodium chlorite, is present in the pharmaceutical composition in an amount ranging from about 1% to about 2% (w/w). In some embodiments, the chlorite salt, e.g., sodium chlorite, is present in the pharmaceutical composition in an amount ranging from about 0.001% to about 1% (w/w). In some embodiments, the chlorite salt, e.g., sodium chlorite, is present in the pharmaceutical composition in an amount ranging from about 0.01% to about 1% (w/w). In some embodiments, the chlorite salt, e.g., sodium chlorite, is present in the pharmaceutical composition in an amount ranging from about 0.1% to about 1% (w/w).

In some aspects, the pharmaceutical composition described herein comprises stabilized chlorine dioxide as a source of sodium chlorite. As used herein “stabilized chlorine dioxide” refers to an aqueous sodium chlorite (NaClO₂) solution. In some embodiments, stabilized chlorine dioxide is prepared by buffering sodium chlorite with a carbonate or a phosphate, and hydrogen peroxide. In some embodiments, stabilized chlorine dioxide further comprises sodium chlorate (NaClO₃). In some embodiments, stabilized chlorine dioxide further comprises sodium chloride (NaCl). In some embodiments, stabilized chlorine dioxide is commercially available and comprises from about 2% to about 4% sodium chlorite. In some embodiments, stabilized chlorine dioxide is commercially available and comprises about 3% sodium chlorite. In some embodiments and under the right pH conditions, stabilized chlorine dioxide further comprises chlorine dioxide (ClO₂). In some embodiments, the oxychlorine-based component of the composition described herein (e.g. sodium chlorite, stabilized chlorine dioxide, or chlorine dioxide) inhibits the cellular protein synthesis. In some embodiments, the oxychlorine-based component of the composition described herein (e.g. sodium chlorite, stabilized chlorine dioxide, or chlorine dioxide) inhibits the destruction of disulfide bonds. In some embodiments, the stabilized chlorine dioxide is present in the pharmaceutical composition in an amount ranging from about 0.005% to about 1% (w/w). In some embodiments, the stabilized chlorine dioxide is present in the pharmaceutical composition in an amount of about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% (w/w). In some embodiments, the stabilized chlorine dioxide is present in the pharmaceutical composition in an amount ranging from about 0.005% to about 0.1% (w/w). In some embodiments, the stabilized chlorine dioxide is present in the pharmaceutical composition in an amount ranging from about 0.01% to about 0.1% (w/w). In some embodiments, the stabilized chlorine dioxide is present in the pharmaceutical composition in an amount ranging from about 0.005% to about 0.01% (w/w). In some embodiments, the stabilized chlorine dioxide is present in the pharmaceutical composition in an amount ranging from about 0.009% to about 0.011% (w/w). In some embodiments, the stabilized chlorine dioxide is present in the pharmaceutical composition in an amount of about 0.01% (w/w). In some embodiments, the stabilized chlorine dioxide is present in the pharmaceutical composition in an amount ranging from about 0.001% to about 0.005% (w/w). In some embodiments, the stabilized chlorine dioxide is present in the pharmaceutical composition in an amount of about 0.02% (w/w). In some embodiments, the stabilized chlorine dioxide is present in the pharmaceutical composition in an amount of about 0.03% (w/w).

In some embodiments, the stabilized chlorine dioxide is present in the pharmaceutical composition in an amount ranging from about 0.0001% to about 10% (w/w). In some embodiments, the stabilized chlorine dioxide is present in the pharmaceutical composition in an amount ranging from about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w). In some embodiments, the stabilized chlorine dioxide is present in the pharmaceutical composition in an amount ranging from about 0.0001% to about 5% (w/w). In some embodiments, the stabilized chlorine dioxide is present in the pharmaceutical composition in an amount ranging from about 0.0001% to about 4% (w/w). In some embodiments, the stabilized chlorine dioxide is present in the pharmaceutical composition in an amount ranging from about 0.0001% to about 3% (w/w). In some embodiments, the stabilized chlorine dioxide is present in the pharmaceutical composition in an amount ranging from about 0.0001% to about 2% (w/w). In some embodiments, the stabilized chlorine dioxide is present in the pharmaceutical composition in an amount ranging from about 0.0001% to about 1% (w/w). In some embodiments, the stabilized chlorine dioxide is present in the pharmaceutical composition in an amount ranging from about 0.0001% to about 0.1% (w/w). In some embodiments, the stabilized chlorine dioxide is present in the pharmaceutical composition in an amount ranging from about 0.0001% to about 0.01% (w/w). In some embodiments, the stabilized chlorine dioxide is present in the pharmaceutical composition in an amount ranging from about 0.0001% to about 0.001% (w/w). In some embodiments, the stabilized chlorine dioxide is present in the pharmaceutical composition in an amount ranging from about 0.001% to about 5% (w/w). In some embodiments, the stabilized chlorine dioxide is present in the pharmaceutical composition in an amount ranging from about 0.001% to about 4% (w/w). In some embodiments, the stabilized chlorine dioxide is present in the pharmaceutical composition in an amount ranging from about 0.001% to about 3% (w/w). In some embodiments, the stabilized chlorine dioxide is present in the pharmaceutical composition in an amount ranging from about 0.001% to about 2% (w/w). In some embodiments, the stabilized chlorine dioxide is present in the pharmaceutical composition in an amount ranging from about 0.001% to about 1% (w/w). In some embodiments, the stabilized chlorine dioxide is present in the pharmaceutical composition in an amount ranging from about 0.001% to about 0.1% (w/w). In some embodiments, the stabilized chlorine dioxide is present in the pharmaceutical composition in an amount ranging from about 0.001% to about 0.01% (w/w). In some embodiments, the stabilized chlorine dioxide is present in the pharmaceutical composition in an amount ranging from about 0.01% to about 5% (w/w). In some embodiments, the stabilized chlorine dioxide is present in the pharmaceutical composition in an amount ranging from about 0.01% to about 4% (w/w). In some embodiments, the stabilized chlorine dioxide is present in the pharmaceutical composition in an amount ranging from about 0.01% to about 3% (w/w). In some embodiments, the stabilized chlorine dioxide is present in the pharmaceutical composition in an amount ranging from about 0.01% to about 2% (w/w). In some embodiments, the stabilized chlorine dioxide is present in the pharmaceutical composition in an amount ranging from about 0.01% to about 1% (w/w). In some embodiments, the stabilized chlorine dioxide is present in the pharmaceutical composition in an amount ranging from about 0.01% to about 0.1% (w/w). In some embodiments, the stabilized chlorine dioxide is present in the pharmaceutical composition in an amount ranging from about 0.001% to about 5% (w/w). In some embodiments, the stabilized chlorine dioxide is present in the pharmaceutical composition in an amount ranging from about 0.01% to about 5% (w/w). In some embodiments, the stabilized chlorine dioxide is present in the pharmaceutical composition in an amount ranging from about 0.1% to about 5% (w/w). In some embodiments, the stabilized chlorine dioxide is present in the pharmaceutical composition in an amount ranging from about 1% to about 5% (w/w). In some embodiments, the stabilized chlorine dioxide is present in the pharmaceutical composition in an amount ranging from about 2% to about 5% (w/w). In some embodiments, the stabilized chlorine dioxide is present in the pharmaceutical composition in an amount ranging from about 3% to about 5% (w/w). In some embodiments, the stabilized chlorine dioxide is present in the pharmaceutical composition in an amount ranging from about 4% to about 5% (w/w). In some embodiments, the stabilized chlorine dioxide is present in the pharmaceutical composition in an amount ranging from about 0.001% to about 4% (w/w). In some embodiments, the stabilized chlorine dioxide is present in the pharmaceutical composition in an amount ranging from about 0.01% to about 4% (w/w). In some embodiments, the stabilized chlorine dioxide is present in the pharmaceutical composition in an amount ranging from about 0.1% to about 4% (w/w). In some embodiments, the stabilized chlorine dioxide is present in the pharmaceutical composition in an amount ranging from about 1% to about 4% (w/w). In some embodiments, the stabilized chlorine dioxide is present in the pharmaceutical composition in an amount ranging from about 2% to about 4% (w/w). In some embodiments, the stabilized chlorine dioxide is present in the pharmaceutical composition in an amount ranging from about 3% to about 4% (w/w). In some embodiments, the stabilized chlorine dioxide is present in the pharmaceutical composition in an amount ranging from about 0.001% to about 3% (w/w). In some embodiments, the stabilized chlorine dioxide is present in the pharmaceutical composition in an amount ranging from about 0.01% to about 3% (w/w). In some embodiments, the stabilized chlorine dioxide is present in the pharmaceutical composition in an amount ranging from about 0.1% to about 3% (w/w). In some embodiments, the stabilized chlorine dioxide is present in the pharmaceutical composition in an amount ranging from about 1% to about 3% (w/w). In some embodiments, the stabilized chlorine dioxide is present in the pharmaceutical composition in an amount ranging from about 2% to about 3% (w/w). In some embodiments, the stabilized chlorine dioxide is present in the pharmaceutical composition in an amount ranging from about 0.001% to about 2% (w/w). In some embodiments, the stabilized chlorine dioxide is present in the pharmaceutical composition in an amount ranging from about 0.01% to about 2% (w/w). In some embodiments, the stabilized chlorine dioxide is present in the pharmaceutical composition in an amount ranging from about 0.1% to about 2% (w/w). In some embodiments, the stabilized chlorine dioxide is present in the pharmaceutical composition in an amount ranging from about 1% to about 2% (w/w). In some embodiments, the stabilized chlorine dioxide is present in the pharmaceutical composition in an amount ranging from about 0.001% to about 1% (w/w). In some embodiments, the stabilized chlorine dioxide is present in the pharmaceutical composition in an amount ranging from about 0.01% to about 1% (w/w). In some embodiments, the stabilized chlorine dioxide is present in the pharmaceutical composition in an amount ranging from about 0.1% to about 1% (w/w).

### Quaternary Ammonium Salt

In some aspects, the pharmaceutical composition comprises a quaternary ammonium salt. In some embodiments, the quaternary ammonium salt is a salt of a quaternary ammonium cation. As used herein “quaternary ammonium cations” also known as quats, refer to positively charged polyatomic ions of the structure NR₄⁺, R being an alkyl group or an aryl group. Unlike the ammonium ion (NH₄⁺) and the primary, secondary, or tertiary ammonium cations, the quaternary ammonium cations are permanently charged, independent of the pH of their solution. In some embodiments, the quaternary ammonium salt is a polymeric quaternary ammonium salt. In some embodiments, the quaternary ammonium salt comprises a C12 or C14 alkyl chain. In some embodiments, the quaternary ammonium salt is not benzalkonium chloride. In some embodiments, the quaternary ammonium salt does not cause sensitivity. In some embodiments, the quaternary ammonium salt is C12-C14-alkyl(ethylbenzyl)dimethylammonium chloride. In some embodiments, the quaternary ammonium salt, e.g., C12-C14-alkyl(ethylbenzyl)dimethylammonium chloride, is present in the pharmaceutical composition in an amount ranging from about 0.0001% to about 0.5% (w/w). In some embodiments, the quaternary ammonium salt, e.g., C12-C14-alkyl(ethylbenzyl)dimethylammonium chloride is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, or about 0.5% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.1% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.01% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.002% (w/w). In some embodiments, the quaternary ammonium salt, e.g., C12-C14-alkyl(ethylbenzyl)dimethylammonium chloride, is present in the pharmaceutical composition in an amount ranging from about 0.0005% to about 0.002% (w/w). In some embodiments, the quaternary ammonium salt, e.g., C12-C14-alkyl(ethylbenzyl)dimethylammonium chloride, is present in the pharmaceutical composition in an amount ranging from about 0.0005% to about 0.0012% (w/w). In some embodiments, the quaternary ammonium salt, e.g., C12-C14-alkyl(ethylbenzyl)dimethylammonium chloride, is present in the pharmaceutical composition in an amount ranging from about 0.0005% to about 0.005% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 0.005% (w/w). In some embodiments, the quaternary ammonium salt, e.g., C12-C14-alkyl(ethylbenzyl)dimethylammonium chloride, is present in the pharmaceutical composition in an amount of about 0.001% (w/w). In some embodiments, the quaternary ammonium salt, e.g., C12-C14-alkyl(ethylbenzyl)dimethylammonium chloride, is present in the pharmaceutical composition in an amount of about 0.0008% (w/w). In some embodiments, the quaternary ammonium salt, e.g., C12-C14-alkyl(ethylbenzyl)dimethylammonium chloride, is present in the pharmaceutical composition in an amount of about 0.003% (w/w).

In some embodiments, the quaternary ammonium salt, e.g., C12-C14-alkyl(ethylbenzyl)dimethylammonium chloride, is present in the pharmaceutical composition in an amount ranging from about 0.0001% to about 10% (w/w). In some embodiments, the quaternary ammonium salt, e.g., C12-C14-alkyl(ethylbenzyl)dimethylammonium chloride, is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 1% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.1% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.01% (w/w). In some embodiments, the quaternary ammonium salt, e.g., C12-C14-alkyl(ethylbenzyl)dimethylammonium chloride, is present in the pharmaceutical composition in an amount ranging from about 0.0001% to about 0.001% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 1% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 0.1% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 0.01% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 1% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 0.1% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.1% to about 5% (w/w). In some embodiments, the quaternary ammonium salt, e.g., C12-C14-alkyl(ethylbenzyl)dimethylammonium chloride, is present in the pharmaceutical composition in an amount ranging from about 1% to about 5% (w/w). In some embodiments, the quaternary ammonium salt, e.g., C12-C14-alkyl(ethylbenzyl)dimethylammonium chloride, is present in the pharmaceutical composition in an amount ranging from about 2% to about 5% (w/w). In some embodiments, the quaternary ammonium salt, e.g., C12-C14-alkyl(ethylbenzyl)dimethylammonium chloride, is present in the pharmaceutical composition in an amount ranging from about 3% to about 5% (w/w). In some embodiments, the quaternary ammonium salt, e.g., C12-C14-alkyl(ethylbenzyl)dimethylammonium chloride, is present in the pharmaceutical composition in an amount ranging from about 4% to about 5% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.1% to about 4% (w/w). In some embodiments, the quaternary ammonium salt, e.g., C12-C14-alkyl(ethylbenzyl)dimethylammonium chloride, is present in the pharmaceutical composition in an amount ranging from about 1% to about 4% (w/w). In some embodiments, the quaternary ammonium salt, e.g., C12-C14-alkyl(ethylbenzyl)dimethylammonium chloride, is present in the pharmaceutical composition in an amount ranging from about 2% to about 4% (w/w). In some embodiments, the quaternary ammonium salt, e.g., C12-C14-alkyl(ethylbenzyl)dimethylammonium chloride, is present in the pharmaceutical composition in an amount ranging from about 3% to about 4% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.1% to about 3% (w/w). In some embodiments, the quaternary ammonium salt, e.g., C12-C14-alkyl(ethylbenzyl)dimethylammonium chloride, is present in the pharmaceutical composition in an amount ranging from about 1% to about 3% (w/w). In some embodiments, the quaternary ammonium salt, e.g., C12-C14-alkyl(ethylbenzyl)dimethylammonium chloride, is present in the pharmaceutical composition in an amount ranging from about 2% to about 3% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.1% to about 2% (w/w). In some embodiments, the quaternary ammonium salt, e.g., C12-C14-alkyl(ethylbenzyl)dimethylammonium chloride, is present in the pharmaceutical composition in an amount ranging from about 1% to about 2% (w/w). In some embodiments, the quaternary ammonium salt, e.g., C12-C14-alkyl(ethylbenzyl)dimethylammonium chloride, is present in the pharmaceutical composition in an amount ranging from about 0.001% to about 1% (w/w). In some embodiments, the quaternary ammonium salt, e.g., C12-C14-alkyl(ethylbenzyl)dimethylammonium chloride, is present in the pharmaceutical composition in an amount ranging from about 0.01% to about 1% (w/w). In some embodiments, the quaternary ammonium salt, e.g., C12-C14-alkyl(ethylbenzyl)dimethylammonium chloride, is present in the pharmaceutical composition in an amount ranging from about 0.1% to about 1% (w/w). In some embodiments, the quaternary ammonium salt destroys phospholipids within the microbial cell wall, prompting autolysis and microbial cell entry for the oxychlorine-based component in the formulation (e.g. sodium chlorite, stabilized chlorine dioxide, or chlorine dioxide).

In some embodiments, the quaternary ammonium salt destroys phospholipids within the microbial cell wall, prompting autolysis and microbial cell entry for the oxychlorine-based component in the formulation (e.g. sodium chlorite, stabilized chlorine dioxide, or chlorine dioxide).

### Ammonium Chloride

In some aspects, the pharmaceutical composition comprises ammonium chloride. As used herein “ammonium chloride” refers to NH₄Cl. In some embodiments, the ammonium chloride is present in the pharmaceutical composition in an amount ranging from about 0.001% to about 2% (w/w). In some embodiments, the ammonium chloride is present in the pharmaceutical composition in an amount of about 0.001%, about 0.005%, about 0.01%, about 0.015%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, about 0.1%, about 0.11%, about 0.12%, about 0.13%, about 0.14%, about 0.15%, about 0.16%, about 0.17%, about 0.18%, about 0.19%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% (w/w). In some embodiments, the ammonium chloride is present in the pharmaceutical composition in an amount ranging from about 0.001% to about 1% (w/w). In some embodiments, the ammonium chloride is present in the pharmaceutical composition in an amount ranging from about 0.01% to about 1% (w/w). In some embodiments, the ammonium chloride is present in the pharmaceutical composition in an amount ranging from about 0.1% to about 1% (w/w). In some embodiments, the ammonium chloride is present in the pharmaceutical composition in an amount ranging from about 0.01% to about 0.3% (w/w). In some embodiments, the ammonium chloride is present in the pharmaceutical composition in an amount ranging from about 0.1% to about 0.3% (w/w). In some embodiments, the ammonium chloride is present in the pharmaceutical composition in an amount of about 0.2% (w/w). In some embodiments, the ammonium chloride is present in the pharmaceutical composition in an amount ranging from about 0.001% to about 0.05% (w/w). In some embodiments, the ammonium chloride is present in the pharmaceutical composition in an amount ranging from about 0.001% to about 0.05% (w/w). In some embodiments, the ammonium chloride is present in the pharmaceutical composition in an amount ranging from about 0.01% to about 0.05% (w/w). In some embodiments, the ammonium chloride is present in the pharmaceutical composition in an amount of about 0.025% (w/w).

In some embodiments, the ammonium chloride is present in the pharmaceutical composition in an amount ranging from about 0.0001% to about 10% (w/w). In some embodiments, the ammonium chloride is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w). In some embodiments, the ammonium chloride is present in the pharmaceutical composition in an amount ranging from about 0.0001% to about 5% (w/w). In some embodiments, the ammonium chloride is present in the pharmaceutical composition in an amount ranging from about 0.0001% to about 4% (w/w). In some embodiments, the ammonium chloride is present in the pharmaceutical composition in an amount ranging from about 0.0001% to about 3% (w/w). In some embodiments, the ammonium chloride is present in the pharmaceutical composition in an amount ranging from about 0.0001% to about 2% (w/w). In some embodiments, the ammonium chloride is present in the pharmaceutical composition in an amount ranging from about 0.0001% to about 1% (w/w). In some embodiments, the ammonium chloride is present in the pharmaceutical composition in an amount ranging from about 0.0001% to about 0.1% (w/w). In some embodiments, the ammonium chloride is present in the pharmaceutical composition in an amount ranging from about 0.0001% to about 0.01% (w/w). In some embodiments, the ammonium chloride is present in the pharmaceutical composition in an amount ranging from about 0.0001% to about 0.001% (w/w). In some embodiments, the ammonium chloride is present in the pharmaceutical composition in an amount ranging from about 0.001% to about 5% (w/w). In some embodiments, the ammonium chloride is present in the pharmaceutical composition in an amount ranging from about 0.001% to about 4% (w/w). In some embodiments, the ammonium chloride is present in the pharmaceutical composition in an amount ranging from about 0.001% to about 3% (w/w). In some embodiments, the ammonium chloride is present in the pharmaceutical composition in an amount ranging from about 0.001% to about 2% (w/w). In some embodiments, the ammonium chloride is present in the pharmaceutical composition in an amount ranging from about 0.001% to about 1% (w/w). In some embodiments, the ammonium chloride is present in the pharmaceutical composition in an amount ranging from about 0.001% to about 0.1% (w/w). In some embodiments, the ammonium chloride is present in the pharmaceutical composition in an amount ranging from about 0.001% to about 0.01% (w/w). In some embodiments, the ammonium chloride is present in the pharmaceutical composition in an amount ranging from about 0.01% to about 5% (w/w). In some embodiments, the ammonium chloride is present in the pharmaceutical composition in an amount ranging from about 0.01% to about 4% (w/w). In some embodiments, the ammonium chloride is present in the pharmaceutical composition in an amount ranging from about 0.01% to about 3% (w/w). In some embodiments, the ammonium chloride is present in the pharmaceutical composition in an amount ranging from about 0.01% to about 2% (w/w). In some embodiments, the ammonium chloride is present in the pharmaceutical composition in an amount ranging from about 0.01% to about 1% (w/w). In some embodiments, the ammonium chloride is present in the pharmaceutical composition in an amount ranging from about 0.01% to about 0.1% (w/w). In some embodiments, the ammonium chloride is present in the pharmaceutical composition in an amount ranging from about 0.001% to about 5% (w/w). In some embodiments, the ammonium chloride is present in the pharmaceutical composition in an amount ranging from about 0.01% to about 5% (w/w). In some embodiments, the ammonium chloride is present in the pharmaceutical composition in an amount ranging from about 0.1% to about 5% (w/w). In some embodiments, the ammonium chloride is present in the pharmaceutical composition in an amount ranging from about 1% to about 5% (w/w). In some embodiments, the ammonium chloride is present in the pharmaceutical composition in an amount ranging from about 2% to about 5% (w/w). In some embodiments, the ammonium chloride is present in the pharmaceutical composition in an amount ranging from about 3% to about 5% (w/w). In some embodiments, the ammonium chloride is present in the pharmaceutical composition in an amount ranging from about 4% to about 5% (w/w). In some embodiments, the ammonium chloride is present in the pharmaceutical composition in an amount ranging from about 0.001% to about 4% (w/w). In some embodiments, the ammonium chloride is present in the pharmaceutical composition in an amount ranging from about 0.01% to about 4% (w/w). In some embodiments, the ammonium chloride is present in the pharmaceutical composition in an amount ranging from about 0.1% to about 4% (w/w). In some embodiments, the ammonium chloride is present in the pharmaceutical composition in an amount ranging from about 1% to about 4% (w/w). In some embodiments, the ammonium chloride is present in the pharmaceutical composition in an amount ranging from about 2% to about 4% (w/w). In some embodiments, the ammonium chloride is present in the pharmaceutical composition in an amount ranging from about 3% to about 4% (w/w). In some embodiments, the ammonium chloride is present in the pharmaceutical composition in an amount ranging from about 0.001% to about 3% (w/w). In some embodiments, the ammonium chloride is present in the pharmaceutical composition in an amount ranging from about 0.01% to about 3% (w/w). In some embodiments, the ammonium chloride is present in the pharmaceutical composition in an amount ranging from about 0.1% to about 3% (w/w). In some embodiments, the ammonium chloride is present in the pharmaceutical composition in an amount ranging from about 1% to about 3% (w/w). In some embodiments, the ammonium chloride is present in the pharmaceutical composition in an amount ranging from about 2% to about 3% (w/w). In some embodiments, the ammonium chloride is present in the pharmaceutical composition in an amount ranging from about 0.001% to about 2% (w/w). In some embodiments, the ammonium chloride is present in the pharmaceutical composition in an amount ranging from about 0.01% to about 2% (w/w). In some embodiments, the ammonium chloride is present in the pharmaceutical composition in an amount ranging from about 0.1% to about 2% (w/w). In some embodiments, the ammonium chloride is present in the pharmaceutical composition in an amount ranging from about 1% to about 2% (w/w). In some embodiments, the ammonium chloride is present in the pharmaceutical composition in an amount ranging from about 0.001% to about 1% (w/w). In some embodiments, the ammonium chloride is present in the pharmaceutical composition in an amount ranging from about 0.01% to about 1% (w/w). In some embodiments, the ammonium chloride is present in the pharmaceutical composition in an amount ranging from about 0.1% to about 1% (w/w).

In some embodiments, ammonium chloride enhances the effectiveness for autolysis on hard-to-kill Gm-bacteria, spore formers, fungi, and recalcitrant organism such as pathogenic amoeba.

### Surfactant

In some aspects, the pharmaceutical composition further comprises a surfactant. In some embodiments, a surfactant is used to disperse insoluble ingredients or to aid in solubilization. In some embodiments, the surfactant is an anionic surfactant. In some embodiments, the surfactant is a cationic surfactant. In some embodiments, the surfactant is a zwitterionic surfactant. In some embodiments, the surfactant is a nonionic surfactant. In some embodiments, the surfactant is selected from sodium lauryl sulfate, docusate sodium, polyoxyalkyl ethers, polyoxylalkyl phenyl ethers, polyoxyl castor oils, polyoxyl hydrogenated castor oils, polyoxyl 40 stearates, polyoxy sorbitan esters, sorbitan esters, polysorbates, sorbitan monolaureates, poloxamines, poloxamers, and any combinations thereof. In some embodiments, the surfactant is a polyoxyethylene-polyoxypropylene block copolymer. In some embodiments, the surfactant is a poloxamer. In some embodiments, the surfactant is a poloxamine. In some embodiments, the surfactant is Tetronic® 908. In some embodiments, the pharmaceutical composition comprises a surfactant, e.g., Tetronic® 908, in an amount ranging from about 0.1% and about 1% (w/w). In some embodiments, the pharmaceutical composition comprises a surfactant, e.g., Tetronic® 908, in an amount of about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, or about 1% (w/w). In some embodiments, the pharmaceutical composition comprises a surfactant, e.g., Tetronic® 908 in an amount of about 0.25% (w/w). In some embodiments, the surfactant is Tetronic® 908. In some embodiments, the pharmaceutical composition comprises a surfactant, e.g., Tetronic® 908, in an amount ranging from about 0.01% and about 0.5% (w/w). In some embodiments, the pharmaceutical composition comprises a surfactant, e.g., Tetronic® 908, in an amount of about 0.01%, about 0.015%, about 0.02%, about 0.025%, about 0.03%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, about 0.1% (w/w), about 0.15% (w/w), about 0.2% (w/w), about 0.25% (w/w), about 0.30% (w/w), about 0.35% (w/w), about 0.4% (w/w), about 0.45% (w/w), or about 0.5% (w/w). In some embodiments, the pharmaceutical composition comprises a surfactant, e.g., Tetronic® 908 in an amount of about 0.02% (w/w).

### Tonicity Agent

In some aspects, the pharmaceutical composition further comprises a tonicity agent. Tonicity refers to the osmotic pressure exerted by salts in aqueous solutions. An ophthalmic solution is isotonic with another solution when the magnitudes of the colligative properties of the solutions are equal. An ophthalmic solution is considered isotonic when its tonicity equal to that of a 0.9% sodium chloride solution (290 mOsm). The osmotic pressure of the aqueous intraocular fluid is slightly higher than that of normal tears, measuring about 305 mOsm. In some embodiments, the tonicity agent is selected from sodium chloride, potassium chloride, mannitol, dextrose, glycerin, propylene glycol, and any combinations thereof. In some embodiments, the pharmaceutical composition comprises a tonicity agent in an amount ranging from about 0.001% to about 1% (w/w). In some embodiments, the pharmaceutical composition comprises a tonicity agent in an amount of about 0.001%, about 0.0015%, about 0.002%, about 0.0025%, about 0.003%, about 0.0035%, about 0.004%, about 0.0045%, about 0.005%, about 0.0055%, about 0.006%, about 0.0065%, about 0.007%, about 0.0075%, about 0.008%, about 0.0085%, about 0.009%, about 0.0095%, about 0.01%, about 0.015%, about 0.02%, about 0.025%, about 0.03%, about 0.035%, about 0.04%, about 0.045%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% (w/w). In some embodiments, the tonicity agent is sodium chloride and is present in an amount ranging from about 0.001% to about 0.1% (w/w). In some embodiments, the tonicity agent is sodium chloride and is present in an amount ranging from about 0.001% to about 0.01% (w/w). In some embodiments, the tonicity agent is sodium chloride and is present in an amount ranging from about 0.005% to about 0.01% (w/w). In some embodiments, the tonicity agent is sodium chloride and is present in an amount ranging from about 0.05% to about 1% (w/w). In some embodiments, the tonicity agent is sodium chloride and is present in an amount of about 0.1% (w/w). In some embodiments, the tonicity agent is potassium chloride and is present in an amount ranging from about 0.05% to about 1% (w/w). In some embodiments, the tonicity agent is potassium chloride and is present in an amount of about 0.2% (w/w). In some embodiments, the tonicity agent is propylene glycol and is present in an amount ranging from about 0.05% to about 1% (w/w). In some embodiments, the tonicity agent is propylene glycol and is present in an amount of about 0.75% (w/w). In some embodiments, the tonicity agent is propylene glycol and is present in an amount ranging from about 0.001% to about 0.1% (w/w). In some embodiments, the tonicity agent is propylene glycol and is present in an amount of about 0.0075% (w/w). In some embodiments, a mixture of sodium chlorite, potassium chloride, and propylene glycol is used as a tonicity agent.

Chelating agent

In some aspects, the pharmaceutical composition further comprises a chelating agent. In some embodiments, the chelating agent is EDTA. In some embodiments, the chelating agent is disodium edetate. In some embodiments, the chelating agent is tetrasodium edetate. In some embodiments, the chelating agent is present in the pharmaceutical composition in an amount ranging from about 0.01% to about 1% (w/w). In some embodiments, the chelating agent is present in the pharmaceutical composition in an amount of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% (w/w). In some embodiments, the chelating agent is EDTA and is present in the pharmaceutical composition in an amount of about 0.05% (w/w). In some embodiments, the chelating agent is disodium edetate and is present in the pharmaceutical composition in an amount of about 0.05% (w/w).

### Buffer

In some aspects, the pharmaceutical composition further comprises a buffer. In some embodiments, a buffer is used to maintain the pH between about 6 and about 8. In some embodiments, a buffer is used to maintain the pH at about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8. In some embodiments, a buffer is used to maintain the pH between about 7 and about 8. In some embodiments, a buffer is used to maintain the pH between about 7 and about 7.4. In some embodiments, a buffer is used to maintain the pH at about 7. In some embodiments, a buffer is used to maintain the pH at a value equivalent to the tear fluid value (7.4). Exemplary buffers include, but are not limited to: phosphate buffer, citrate buffer, acetate buffer, sulfonate buffer, borate buffer, and any combinations thereof. In some embodiments, the buffer is a phosphate buffer. In some embodiments, the buffer comprises sodium phosphate monobasic (monohydrate) in an amount ranging from about 0.005% to about 0.5% (w/w). In some embodiments, the buffer comprises sodium phosphate monobasic (monohydrate) in an amount of about 0.012% (w/w). In some embodiments, the buffer comprises sodium phosphate dibasic (heptahydrate) in an amount ranging from about 0.005% to about 0.5% (w/w). In some embodiments, the buffer comprises sodium phosphate dibasic (heptahydrate) in an amount of about 0.195% (w/w).

### Viscosity Agent

In some aspects, the pharmaceutical composition further comprises a viscosity agent. Viscosity agents are used in ophthalmic solutions to increase their viscosity. This enables the formulation to remain in the eye longer and gives more time for the drug to exert its therapeutic activity or undergo absorption. The viscosity of a solution is given in poise units. The unit centipoise (cp or the plural cps) is equal to 0.01 poise and is most often used in pharmaceutical applications. Compounds used to enhance viscosity are available in various grades such as 15 cps, 100 cps, etc. In some embodiments, the desired viscosity in an ophthalmic solution is between 25 and 50 cps. The actual concentration of the enhancer required to produce that viscosity will depend on the grade of the viscosity agent. Examples of viscosity agents include, but are not limited to polyvinylalcohol (PVA), polyvinylpyrrolidone (PVP), polyethylene glycol (PEG), polyacrylic acid (PAA), cellulose derivatives (such as hydroxyethylcellulose, hydroxypropylmethylcellulose, and methylcellulose), and any combinations thereof. In some embodiments, the viscosity agent is present in the pharmaceutical composition in an amount ranging from about 0.1% to about 5% (w/w). In some embodiments, the viscosity agent is present in the pharmaceutical composition in an amount of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% (w/w).

### Preservatives

In some aspects, the pharmaceutical composition further comprises a preservative. Ophthalmic solutions are generally packaged in multiple dose containers. In some embodiments, a preservative is added since there is the possibility of inadvertent bacterial contamination of the formulation with repeated patient use. In some embodiments, the preservative used does not cause patient sensitivity or is not incompatible with the other ingredients in the pharmaceutical composition. Examples of preservatives include, but are not limited to benzalkonium chloride, benzethonium chloride, chlorobutanol, phenylmercuric acetate, phenylmercuric nitrate, thimerosal, methylparaben, propylparaben, and any combinations thereof. In some embodiments, the preservative is not benzalkonium chloride. In some embodiments, the preservative is present in the pharmaceutical composition in an amount ranging from about 0.001% to about 1% (w/w). In some embodiments, the viscosity agent is present in the pharmaceutical composition in an amount of about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% (w/w).

### Non-Aqueous Solvent

In some aspects, the pharmaceutical composition further comprises a non-aqueous solvent. Examples of non-aqueous solvents include, but are not limited to, vegetable oils, C₁₄-C₂₀ fatty acid esters, C₆-C₁₂ fatty acid esters of glycerol, and any combinations thereof. In some embodiments, the non-aqueous solvent is a vegetable oil selected from castor oil, coconut oil, cinnamon oil, corn oil, olive oil, cotton seed oil, soybean oil, and any combinations thereof. In some embodiments, the non-aqueous is a C₁₄-C₂₀ fatty acid ester selected from lauric acid, myristic acid, palmitic acid, palmitoleic acid, stearic acid, oleic acid, linoleic acid, α-linolenic acid, y-linolenic acid, eicosapentaenoic acid, ethyl oleate, isopropyl myristate, and any combinations thereof. In some embodiments, the non-aqueous solvent is a C₆-C₁₂ fatty acid ester of glycerol selected from Labrafac® PG, Labrafac® lipophile WL 1349, Miglyol® 812, Miglyol® 818, and any combinations thereof. In some embodiments, the non-aqueous solvent is present in the pharmaceutical composition in an amount ranging from about 0.01% to about 10 (w/v), about 0.1% to about 5% (w/v), or about 0.1% to about 2.5% (w/v).

### pH Adjusting Agent

In some aspects, the pharmaceutical composition further comprises a pH adjusting agent. In some embodiments, the pH adjusting agent is hydrochloric acid. In some embodiments, the pH adjusting agent is sodium hydroxide.

### Antioxidant

In some aspects, the pharmaceutical composition further comprises an antioxidant. Examples of antioxidants include, but are not limited to sodium bisulfite, sodium metabisulfite, thiourea, and any combinations thereof. In some embodiments, the antioxidant is present in the pharmaceutical composition in an amount ranging from about 0.01% to about 1% (w/w). In some embodiments, the antioxidant is present in the pharmaceutical composition in an amount of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% (w/w).

### Dosage Forms

Disclosed herein are dosage forms that are applied topically. In some embodiments, the pharmaceutical composition is applied to the eye by topical administration. In some embodiments, the pharmaceutical composition is applied to the eye and is not in contact with a contact lens before administration. In some embodiments, the pharmaceutical composition is applied to the eye and is not in contact with a contact lens after administration. Also disclosed herein are dosage forms that are injected. In some embodiments, the pharmaceutical composition is injected into the eye by an intravitreal injection. In some embodiments, the pharmaceutical composition is injected into the eye and is not in contact with a contact lens before administration. In some embodiments, the pharmaceutical composition is injected into the eye and is not in contact with a contact lens after administration.

In some aspects, the pharmaceutical composition is in the form of a solution, an emulsion, a solid dispersion, a colloidal dispersion, a suspension, a gel, an ointment, a cream, a lyophilized powder, a nanoparticle formulation, intravitreal injection, or any combinations thereof. In some embodiments, the pharmaceutical composition is impregnated onto a cloth, a wipe, a pad, a sponge, a brush, or a cotton tipped applicator. In some embodiments, the pharmaceutical composition is applied with a cloth, a wipe, a pad, a sponge, a brush, or a cotton tipped applicator. In some embodiments, the pharmaceutical composition is in the form of a surgical wash or an irrigation solution. Exemplary dosage forms of the disclosure include those found in Remington: The Science and Practice of Pharmacy, Twenty Second Ed. (London, UK: Pharmaceutical Press, 2013) incorporated herein by reference for such disclosure.

In some embodiments the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) a chlorite salt; (b) a quaternary ammonium salt; and (c) ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.0003% (w/w) of a chlorite salt; (b) about 0.001% (w/w) of a quaternary ammonium salt; and (c) about 0.2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.0003% (w/w) of sodium chlorite; (b) about 0.001% (w/w) of a quaternary ammonium salt; and (c) about 0.2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.01% (w/w) of stabilized chlorine dioxide; (b) about 0.001% (w/w) of a quaternary ammonium salt; and (c) about 0.2% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.02% (w/w) of stabilized chlorine dioxide; (b) about 0.001% (w/w) of a quaternary ammonium salt; and (c) about 0.025% (w/w) of ammonium chloride. In some embodiments, the pharmaceutical composition in the form of a solution, an emulsion, a solid dispersion, a colloidal dispersion, a suspension, a gel, an ointment, a cream, a lyophilized powder, a nanoparticle formulation, intravitreal injection, or any combinations thereof comprises (a) about 0.03% (w/w) of stabilized chlorine dioxide; (b) about 0.003% (w/w) of a quaternary ammonium salt; and (c) about 0.025% (w/w) of ammonium chloride.

In some embodiments the pharmaceutical composition is applied with a pad, cloth, wipe, sponge, brush, or cotton tipped applicator, or any combinations thereof. In some embodiments the pharmaceutical composition is applied with a pad, cloth, wipe, sponge, brush, or cotton tipped applicator, or any combinations thereof comprises (a) a chlorite salt; (b) a quaternary ammonium salt; and (c) ammonium chloride. In some embodiments, the pharmaceutical composition is applied with a pad, cloth, wipe, sponge, brush, or cotton tipped applicator, or any combinations thereof comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments, the pharmaceutical composition is applied with a pad, cloth, wipe, sponge, brush, or cotton tipped applicator, or any combinations thereof comprises (a) about 0.0003% (w/w) of a chlorite salt; (b) about 0.001% (w/w) of a quaternary ammonium salt; and (c) about 0.2% (w/w) of ammonium chloride. In some embodiments, the pharmaceutical composition is applied with a pad, cloth, wipe, sponge, brush, or cotton tipped applicator, or any combinations thereof comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments, the pharmaceutical composition is applied with a pad, cloth, wipe, sponge, brush, or cotton tipped applicator, or any combinations thereof comprises (a) about 0.0003% (w/w) of sodium chlorite; (b) about 0.001% (w/w) of a quaternary ammonium salt; and (c) about 0.2% (w/w) of ammonium chloride. In some embodiments, the pharmaceutical composition is applied with a pad, cloth, wipe, sponge, brush, or cotton tipped applicator, or any combinations thereof comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments, the pharmaceutical composition is applied with a pad, cloth, wipe, sponge, brush, or cotton tipped applicator, or any combinations thereof comprises (a) about 0.01% (w/w) of stabilized chlorine dioxide; (b) about 0.001% (w/w) of a quaternary ammonium salt; and (c) about 0.2% (w/w) of ammonium chloride. In some embodiments, the pharmaceutical composition is applied with a pad, cloth, wipe, sponge, brush, or cotton tipped applicator, or any combinations thereof comprises (a) about 0.02% (w/w) of stabilized chlorine dioxide; (b) about 0.001% (w/w) of a quaternary ammonium salt; and (c) about 0.025% (w/w) of ammonium chloride. In some embodiments, the pharmaceutical composition is applied with a pad, cloth, wipe, sponge, brush, or cotton tipped applicator, or any combinations thereof comprises (a) about 0.03% (w/w) of stabilized chlorine dioxide; (b) about 0.003% (w/w) of a quaternary ammonium salt; and (c) about 0.025% (w/w) of ammonium chloride. In some embodiments the pharmaceutical composition is impregnated on a pad, cloth, wipe, sponge, brush, or cotton tipped applicator, or any combinations thereof. In some embodiments the pharmaceutical composition is impregnated on a pad, cloth, wipe, sponge, brush, or cotton tipped applicator, or any combinations thereof comprises (a) a chlorite salt; (b) a quaternary ammonium salt; and (c) ammonium chloride. In some embodiments, the pharmaceutical composition is impregnated on a pad, cloth, wipe, sponge, brush, or cotton tipped applicator, or any combinations thereof comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments, the pharmaceutical composition is impregnated on a pad, cloth, wipe, sponge, brush, or cotton tipped applicator, or any combinations thereof comprises (a) about 0.0003% (w/w) of a chlorite salt; (b) about 0.001% (w/w) of a quaternary ammonium salt; and (c) about 0.2% (w/w) of ammonium chloride. In some embodiments, the pharmaceutical composition is impregnated on a pad, cloth, wipe, sponge, brush, or cotton tipped applicator, or any combinations thereof comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments, the pharmaceutical composition is impregnated on a pad, cloth, wipe, sponge, brush, or cotton tipped applicator, or any combinations thereof comprises (a) about 0.0003% (w/w) of sodium chlorite; (b) about 0.001% (w/w) of a quaternary ammonium salt; and (c) about 0.2% (w/w) of ammonium chloride. In some embodiments, the pharmaceutical composition is impregnated on a pad, cloth, wipe, sponge, brush, or cotton tipped applicator, or any combinations thereof comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments, the pharmaceutical composition is impregnated on a pad, cloth, wipe, sponge, brush, or cotton tipped applicator, or any combinations thereof comprises (a) about 0.01% (w/w) of stabilized chlorine dioxide; (b) about 0.001% (w/w) of a quaternary ammonium salt; and (c) about 0.2% (w/w) of ammonium chloride. In some embodiments, the pharmaceutical composition is impregnated on a pad, cloth, wipe, sponge, brush, or cotton tipped applicator, or any combinations thereof comprises (a) about 0.02% (w/w) of stabilized chlorine dioxide; (b) about 0.001% (w/w) of a quaternary ammonium salt; and (c) about 0.025% (w/w) of ammonium chloride. In some embodiments, the pharmaceutical composition is impregnated on a pad, cloth, wipe, sponge, brush, or cotton tipped applicator, or any combinations thereof comprises (a) about 0.03% (w/w) of stabilized chlorine dioxide; (b) about 0.003% (w/w) of a quaternary ammonium salt; and (c) about 0.025% (w/w) of ammonium chloride.

In some embodiments the pharmaceutical composition is in the form of a surgical wash or an irrigation solution. In some embodiments the pharmaceutical composition in the form of a surgical wash or an irrigation solution comprises (a) a chlorite salt; (b) a quaternary ammonium salt; and (c) ammonium chloride. In some embodiments the pharmaceutical composition in the form of a surgical wash or an irrigation solution comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments the pharmaceutical composition in the form of a surgical wash or an irrigation solution comprises (a) about 0.0003% (w/w) of a chlorite salt; (b) about 0.001% (w/w) of a quaternary ammonium salt; and (c) about 0.2% (w/w) of ammonium chloride. In some embodiments the pharmaceutical composition in the form of a surgical wash or an irrigation solution comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments the pharmaceutical composition in the form of a surgical wash or an irrigation solution comprises (a) about 0.0003% (w/w) of sodium chlorite; (b) about 0.001% (w/w) of a quaternary ammonium salt; and (c) about 0.2% (w/w) of ammonium chloride. In some embodiments the pharmaceutical composition in the form of a surgical wash or an irrigation solution comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments the pharmaceutical composition in the form of a surgical wash or an irrigation solution comprises about 0.01% (w/w) of stabilized chlorine dioxide; (b) about 0.001% (w/w) of a quaternary ammonium salt; and (c) about 0.2% (w/w) of ammonium chloride. In some embodiments, the pharmaceutical composition in the form of a surgical wash or an irrigation solution comprises (a) about 0.02% (w/w) of stabilized chlorine dioxide; (b) about 0.001% (w/w) of a quaternary ammonium salt; and (c) about 0.025% (w/w) of ammonium chloride. In some embodiments, the pharmaceutical composition in the form of a surgical wash or an irrigation solution comprises (a) about 0.03% (w/w) of stabilized chlorine dioxide; (b) about 0.003% (w/w) of a quaternary ammonium salt; and (c) about 0.025% (w/w) of ammonium chloride.

### Solutions

In some aspects, the pharmaceutical composition is formulated as an ophthalmic solution. Ophthalmic solutions are sterile solutions intended for instillation in the eye. Included in this dosage form category are solid preparations that, when reconstituted according to the label instructions, result in a solution. In addition to sterility, these dosage forms require the careful consideration of such other pharmaceutical factors as the need for antimicrobial agents, osmolarity, buffering, viscosity, and proper packaging. The corneal contact time of topical ophthalmic solutions increases with the viscosity of the formulations up to 20 centipoise (cP). Further increases result in reflex tearing and blinking in order to regain the original viscosity of the lacrimal fluid (1.05-5.97 cP). The bioavailability increase associated with this longer precorneal permanence allows the frequency of composition application to be reduced. Synthetic polymers, such as polyvinylalcohol (PVA), polyvinylpyrrolidone (PVP), polyethylene glycol (PEG), polyacrylic acid (PAA), and many cellulose derivatives, are commonly employed as viscosity enhancers because of their physiologic compatibility and satisfactory physicochemical properties. A more sophisticated approach consists of using polymers that provide the liquid formulation with semisolid consistency only when it is placed in the conjunctival or corneal area. In this way, easy instillation of the solution is followed by prolonged permanence as a result of the viscoelastic properties of the formed hydrogel. This in situ gelling phenomenon is caused by a change in the conformation of the polymer(s) that can be triggered by external stimuli such as temperature, pH, ionic content, and lacrimal fluid upon delivery into the eye. Additionally, some polymers can interact, via noncovalent bonds, with conjunctival mucin and maintain the formulations in contact with corneal tissues until mucin turnover leads to their removal.

### Suspensions

In some aspects, the pharmaceutical composition is formulated as an ophthalmic suspension. Ophthalmic suspensions are used to increase the corneal contact time of the active ingredient(s) and thus provide a more sustained action. Included in this dosage form category are those solid preparations that, when reconstituted according to the label instructions, result in a suspension. In some embodiments, the ophthalmic suspension is required when the active ingredient is insoluble in the desired vehicle or is unstable in solution. In some embodiments, the suspensions are prepared with the insoluble active ingredient(s) in a micronized form to prevent irritation or scratching of the cornea. Suspensions are commonly formulated by dispersing micronized active ingredient(s) powder (less than 10 μm in diameter) in a suitable aqueous vehicle. Ophthalmic suspensions are thought to be acceptable as delivery systems since it is assumed that active ingredient(s) particles persist in the conjunctival sac giving rise to a sustained-release effect. Particle size in suspensions for ocular active ingredient(s) delivery is important. An increase in active ingredient(s) particle size enhances the ocular bioavailability. In some embodiments, a particle size above 10 μm in diameter results in a foreign body sensation in the eye following ocular application, causing reflex tearing. A reduction in particle size generally improves the patient comfort and acceptability of suspension formulations. In some embodiments, surfactants are included in the ophthalmic suspension to disperse the active ingredient(s) effectively during manufacture and during product use. Is some embodiments, nonionic surfactants are used because they tend to be less toxic. The level of surfactant included in the formulation should be carefully evaluated, as excessive amounts can lead to irritation in the eye, foaming during manufacture and upon shaking the product, or interactions with other excipients. Consideration must be given to establishing good physical stability of a suspension. If the particles settle and eventually produce a cake at the bottom of the container, they must redisperse readily to achieve dosage uniformity. In some embodiments, viscosity-enhancing agents are used to keep the particles suspended. Ophthalmic suspensions possess the same characteristic of sterility as ophthalmic solutions, with proper consideration given also to preservation, osmolarity, buffering, viscosity, and packaging. In some embodiments, the ophthalmic suspension contains particles of such chemical characteristics and small dimensions that they are nonirritating to the eyes. In some embodiments, the ophthalmic suspension is formulated so that the suspended particles do not agglomerate into larger ones upon storage. In some embodiments, sterile powders for reconstitution (resulting in a solution or suspension, after reconstitution) are used for active ingredient(s) that have limited stability in liquid form. In some embodiments, the sterile powder is manufactured by lyophilization in the individual container. In some embodiments, the composition in powdered form has a much longer shelf life than in solution or suspension.

### Ointments

In some aspects, the pharmaceutical composition is formulated as an ophthalmic ointment. Ophthalmic ointments are sterile. In some embodiments, the ointment is terminally sterilized, or, alternatively, is manufactured from sterile ingredients in an aseptic environment. In some embodiments, filtration through a suitable membrane or dry heat sterilization is used. The ointment base selected for an ophthalmic ointment must be nonirritating to the eye and must permit the diffusion of the active ingredient(s) throughout the secretions bathing the eye. Ointment bases utilized for ophthalmics have a melting or softening point close to body temperature. Ophthalmic ointments have a longer ocular contact time when compared to many ophthalmic solutions. Studies have shown that the ocular contact time is two to four times greater when ointments are used than when a saline solution is used.

### Gels

In some aspects, the pharmaceutical composition is formulated as an ophthalmic gel. Ophthalmic gels are composed of mucoadhesive polymers that provide localized delivery of an active ingredient to the eye. Such polymers have a property known as bioadhesion meaning attachment of a drug carrier to a specific biological tissue. These polymers are able to extend the contact time of the active ingredient(s) with the biological tissues and thereby improve ocular bioavailability. The choice of the polymer plays a critical role in the release kinetics of the active ingredient(s) from the dosage form. Several bioadhesive polymers are available with varying degree of mucoadhesive performance. Some examples are carboxymethylcellulose, carbopol, polycarbophil, and sodium alginate.

### Emulsions

In some aspects, the pharmaceutical composition is formulated as an ophthalmic emulsion. Ophthalmic emulsions are prepared by dissolving or dispersing the active ingredient(s) into an oil phase, adding suitable emulsifying and suspending agents and mixing with water vigorously to form a uniform oil-in-water emulsion. Each phase is typically sterilized prior to or during charging into the mixing vessel. In some embodiments, high-shear homogenation is employed to reduce oil droplet size to sub-micron size which may improve the physical stability of the oil micelles so they do not coalesce. In some embodiments, the resulting dosage form contains small oil droplets, uniformly suspended. In some embodiments, the active ingredient(s) are added to the phase in which it is soluble at the beginning of the manufacturing process, or they added after the emulsion is prepared by a suitable dispersion process. To prevent flocculation, creaming, and coalescence of the emulsions, manufacturers commonly add surfactants to increase the kinetic stability of the emulsion so that the emulsion does not change significantly with time. In some embodiments, emulsions exhibit three types of instability: flocculation, creaming, and coalescence. Flocculation describes the process by which the dispersed phase comes out of suspension in the form of flakes. Emulsions can also undergo creaming, where one of the phases migrate to the top (or the bottom, depending on the relative densities of the two phases) of the emulsion. Coalescence is another form of instability in which small droplets within the media continuously combine to form progressively larger droplets.

### Inserts

In some aspects, the pharmaceutical composition is formulated as an ophthalmic insert. Ophthalmic inserts and ocular systems are solid dosage forms of appropriate size and shape that are placed in the conjunctival fornix, in the lachrymal punctum, or on the cornea. They can be classified as erodible (soluble) and nonerodible (insoluble). These devices allow for accurate dose delivery and can notably increase ocular bioavailability. In some embodiments, the active ingredient(s) release from soluble inserts involves two steps: (1) fast release of a portion of the active ingredient(s) as the tear fluid penetrates into the system; and (2) slow release as a gel layer is formed on the surface of the insert. Collagen shields made from porcine sclera collagen or bovine corium tissue and devices obtained by molding, extrusion, or compression (minitablets) of gelling polymers belong to this category. In some embodiments, bioerodible polymers (e.g. crosslinked gelatin derivatives, poly vinyl alcohol, hypromellose, and polyesters) are used to prepare erodible inserts. These matrices act as simple reservoirs or interact with the active ingredient(s) through labile bonds; the ease with which these bonds can be broken regulates release of the active ingredient(s). In some embodiments, the matrices dissolve within 12-24 h. In some embodiments, insoluble inserts have a reservoir or matrix structure. They release the active ingredient(s) for longer periods of time.

### Intravitreal Injections

Intravitreal injection is a method of delivering a therapeutic agent directly to an interior portion of the eye by using a needle passing through the pars plana. As a parenteral formulation with additional constraints dictated by this delivery route, multiple features have to be balanced in order to develop a successful formulation. Some of these considerations included low dosing volumes (≤0.1 mL), a limited repertoire of safe excipients for intravitreal injection, and the unique physical chemical properties of the active ingredient(s).

### Stability

Disclosed herein are stable pharmaceutical compositions. The pharmaceutical compositions described herein are stable in various storage conditions including refrigerated, ambient, and accelerated conditions.

In some embodiments, stable as used herein refers to a pharmaceutical composition having about 5% w/w or less total impurities at the end of a given storage period. Stability is assessed by HPLC or any other known testing method. In some embodiments, the stable pharmaceutical composition has about 5% w/w, about 4% w/w, about 3% w/w, about 2.5% w/w, about 2% w/w, about 1.5% w/w, about 1% w/w, or about 0.5% w/w total impurities at the end of a given storage period. In other embodiments, the pharmaceutical composition has about 5% w/w total impurities. In yet other embodiments, the pharmaceutical composition has about 4% w/w total impurities. In yet other embodiments, pharmaceutical composition has about 3% w/w total impurities. In yet other embodiments, the pharmaceutical composition has about 2% w/w total impurities. In yet other embodiments, the pharmaceutical composition has about 1% w/w total impurities. In yet other embodiments, the pharmaceutical composition has about 0.9% w/w total impurities. In yet other embodiments, the pharmaceutical composition has about 0.8% w/w total impurities. In yet other embodiments, the pharmaceutical composition has about 0.7% w/w total impurities. In yet other embodiments, the pharmaceutical composition has about 0.6% w/w total impurities. In yet other embodiments, the pharmaceutical composition has about 0.5% w/w total impurities. In yet other embodiments, the pharmaceutical composition has about 0.4% w/w total impurities. In yet other embodiments, the pharmaceutical composition has about 0.3% w/w total impurities. In yet other embodiments, the pharmaceutical composition has about 0.2% w/w total impurities. In yet other embodiments, the pharmaceutical composition has about 0.1% w/w total impurities. In yet other embodiments, the pharmaceutical composition has about 0.09% w/w total impurities. In yet other embodiments, the pharmaceutical composition has about 0.08% w/w total impurities. In yet other embodiments, the pharmaceutical composition has about 0.07% w/w total impurities. In yet other embodiments, the pharmaceutical composition has about 0.06% w/w total impurities. In yet other embodiments, the pharmaceutical composition has about 0.05% w/w total impurities. In yet other embodiments, the pharmaceutical composition has about 0.04% w/w total impurities. In yet other embodiments, the pharmaceutical composition has about 0.03% w/w total impurities. In yet other embodiments, the pharmaceutical composition has about 0.02% w/w total impurities. In yet other embodiments, the pharmaceutical composition has about 0.01% w/w total impurities. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

In some embodiments, stable as used herein refers to a pharmaceutical composition having about 10% or less loss of biocidal activity at the end of a given storage period. Biocidal activity is assessed by known testing method. In some embodiments, the stable pharmaceutical composition has about 10%, about 9.5%, about 9%, about 8.5%, about 8%, about 7.5%, about 7%, about 6.5%, about 6%, about 5.5%, about 5%, about 4.5%, about 4%, about 3.5%, about 3%, about 2.5%, about 2%, about 1.5%, about 1%, or about 0.5% loss of biocidal activity at the end of a given storage period. In some embodiments, the stable pharmaceutical composition has zero loss of biocidal activity at the end of a given storage period. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

In some embodiments, stable as used herein refers to a pharmaceutical composition having no sign of precipitation at the end of a given storage period. Precipitation is assessed by known testing method. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

### Method of Treatment

Also disclosed herein are methods of treating or preventing an eye condition in a subject in need thereof, the method comprising administering to the subject in need thereof a pharmaceutical composition described herein. In some embodiments of a method of treating or preventing an eye condition in a subject in need thereof, the pharmaceutical composition comprises (a) a chlorite salt; (b) a quaternary ammonium salt; and (c) ammonium chloride. In some embodiments of a method of treating or preventing an eye condition in a subject in need thereof, the pharmaceutical composition comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments of a method of treating or preventing an eye condition in a subject in need thereof, the pharmaceutical composition comprises (a) about 0.0003% (w/w) of a chlorite salt; (b) about 0.001% (w/w) of a quaternary ammonium salt; and (c) about 0.2% (w/w) of ammonium chloride. In some embodiments of a method of treating or preventing an eye condition in a subject in need thereof, the pharmaceutical composition comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments of a method of treating or preventing an eye condition in a subject in need thereof, the pharmaceutical composition comprises (a) about 0.0003% (w/w) of sodium chlorite; (b) about 0.001% (w/w) of a quaternary ammonium salt; and (c) about 0.2% (w/w) of ammonium chloride. In some embodiments of a method of treating or preventing an eye condition in a subject in need thereof, the pharmaceutical composition comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments of a method of treating or preventing an eye condition in a subject in need thereof, the pharmaceutical composition comprises (a) about 0.01% (w/w) of stabilized chlorine dioxide; (b) about 0.001% (w/w) of a quaternary ammonium salt; and (c) about 0.2% (w/w) of ammonium chloride. In some embodiments of a method of treating or preventing an eye condition in a subject in need thereof, the pharmaceutical composition comprises (a) about 0.02% (w/w) of stabilized chlorine dioxide; (b) about 0.001% (w/w) of a quaternary ammonium salt; and (c) about 0.025% (w/w) of ammonium chloride. In some embodiments of a method of treating or preventing an eye condition in a subject in need thereof, the pharmaceutical composition comprises (a) about 0.03% (w/w) of stabilized chlorine dioxide; (b) about 0.003% (w/w) of a quaternary ammonium salt; and (c) about 0.025% (w/w) of ammonium chloride.

In some embodiments of a method of treating or preventing an eye condition in a subject in need thereof, the eye condition is an inflammation. In some embodiments of a method of treating or preventing an eye condition in a subject in need thereof, the inflammation is inflammation of the eyelid. In some embodiments of a method of treating or preventing an eye condition in a subject in need thereof, the inflammation of the eyelid is blepharitis. In some embodiments of a method of treating or preventing an eye condition in a subject in need thereof, the blepharitis is associated with an internal hordeolum. In some embodiments of a method of treating or preventing an eye condition in a subject in need thereof, the blepharitis is associated with an external hordeolum (stye). In some embodiments of a method of treating or preventing an eye condition in a subject in need thereof, the blepharitis is associated with a chalazion. In some embodiments, the blepharitis is posterior blepharitis. In some embodiments of a method of treating or preventing an eye condition in a subject in need thereof, the blepharitis is anterior blepharitis. In some embodiments of a method of treating or preventing an eye condition in a subject in need thereof, the inflammation of the eyelid is meibomianitis. In some embodiments of a method of treating or preventing an eye condition in a subject in need thereof, the inflammation is inflammation of the interior of the eye. In some embodiments of a method of treating or preventing an eye condition in a subject in need thereof, the inflammation is endophthalmitis. In some embodiments of a method of treating or preventing an eye condition in a subject in need thereof, the inflammation is inflammation of the conjuctiva. In some embodiments of a method of treating or preventing an eye condition in a subject in need thereof, the inflammation is conjunctivitis. In some embodiments of a method of treating or preventing an eye condition in a subject in need thereof, the inflammation is inflammation of the cornea. In some embodiments of a method of treating or preventing an eye condition in a subject in need thereof, the inflammation is keratitis. In some embodiments of a method of treating or preventing an eye condition in a subject in need thereof, the inflammation is caused by a bacterial infection. In some embodiments of a method of treating or preventing an eye condition in a subject in need thereof, the inflammation is caused by a viral infection. In some embodiments of a method of treating or preventing an eye condition in a subject in need thereof, the inflammation is caused by a fungal infection. In some embodiments of a method of treating or preventing an eye condition in a subject in need thereof, the inflammation is caused by a parasitic infection. In some embodiments of a method of treating or preventing an eye condition in a subject in need thereof, the inflammation is caused by an amoebic infection.

Also disclosed herein are methods of treating or preventing an infection in a subject in need thereof, the method comprising administering to the subject in need thereof a pharmaceutical composition described herein. In some embodiments of a method of treating or preventing an infection in a subject in need thereof, the pharmaceutical composition comprises (a) a chlorite salt; (b) a quaternary ammonium salt; and (c) ammonium chloride. In some embodiments of a method of treating or preventing an infection in a subject in need thereof, the pharmaceutical composition comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments of a method of treating or preventing an infection in a subject in need thereof, the pharmaceutical composition comprises (a) about 0.0003% (w/w) of a chlorite salt; (b) about 0.001% (w/w) of a quaternary ammonium salt; and (c) about 0.2% (w/w) of ammonium chloride. In some embodiments of a method of treating or preventing an infection in a subject in need thereof, the pharmaceutical composition comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments of a method of treating or preventing an infection in a subject in need thereof, the pharmaceutical composition comprises (a) about 0.0003% (w/w) of sodium chlorite; (b) about 0.001% (w/w) of a quaternary ammonium salt; and (c) about 0.2% (w/w) of ammonium chloride. In some embodiments of a method of treating or preventing an infection in a subject in need thereof, the pharmaceutical composition comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments of a method of treating or preventing an infection in a subject in need thereof, the pharmaceutical composition comprises (a) about 0.01% (w/w) of stabilized chlorine dioxide; (b) about 0.001% (w/w) of a quaternary ammonium salt; and (c) about 0.2% (w/w) of ammonium chloride. In some embodiments of a method of treating or preventing an infection in a subject in need thereof, the pharmaceutical composition comprises (a) about 0.02% (w/w) of stabilized chlorine dioxide; (b) about 0.001% (w/w) of a quaternary ammonium salt; and (c) about 0.025% (w/w) of ammonium chloride. In some embodiments of a method of treating or preventing an infection in a subject in need thereof, the pharmaceutical composition comprises (a) about 0.03% (w/w) of stabilized chlorine dioxide; (b) about 0.003% (w/w) of a quaternary ammonium salt; and (c) about 0.025% (w/w) of ammonium chloride. In some embodiments of a method of treating or preventing an infection in a subject in need thereof, the infection is a bacterial infection. In some embodiments of a method of treating or preventing an infection in a subject in need thereof, the infection is a fungal infection. In some embodiments of a method of treating or preventing an infection in a subject in need thereof, the infection is a mycobacterial infection. In some embodiments of a method of treating or preventing an infection in a subject in need thereof, the infection is a mycoplasmal infection. In some embodiments of a method of treating or preventing an infection in a subject in need thereof, the infection is a viral infection. In some embodiments of a method of treating or preventing an infection in a subject in need thereof, the infection is caused by a protozoan microorganism. In some embodiments of a method of treating or preventing an infection in a subject in need thereof, the infection is caused by a microorganism, which includes but is not limited to organisms under the classification Kingdom Monera, Fungi, Protista, and viruses. In some embodiments of a method of treating or preventing an infection in a subject in need thereof, the infection is an eye infection. In some embodiments of a method of treating or preventing an infection in a subject in need thereof, the pharmaceutical composition described herein is applied to the eye topically. In some embodiments of a method of treating or preventing an infection in a subject in need thereof, the pharmaceutical composition described herein is applied to the eye topically and is not in contact with a contact lens prior to administration. In some embodiments of a method of treating or preventing an infection in a subject in need thereof, the pharmaceutical composition described herein is applied to the eye topically and is not in contact with a contact lens after administration. In some embodiments of a method of treating or preventing an infection in a subject in need thereof, the pharmaceutical composition described herein is applied to the eye via an intravitreal injection. In some embodiments of a method of treating or preventing an infection in a subject in need thereof, the pharmaceutical composition described herein is applied to the eye via an intravitreal injection and is not in contact with a contact lens prior to administration. In some embodiments of a method of treating or preventing an infection in a subject in need thereof, the pharmaceutical composition described herein is applied to the eye via an intravitreal injection and is not in contact with a contact lens after administration. In some embodiments of a method of treating or preventing an infection in a subject in need thereof, the infection is keratitis. In some embodiments of a method of treating or preventing an infection in a subject in need thereof, the infection is conjunctivitis.

In some embodiments of a method of treating or preventing an infection in a subject in need thereof, the infection is a bacterial infection. In some embodiments of a method of treating or preventing an infection in a subject in need thereof, the infection is a fungal infection. In some embodiments of a method of treating or preventing an infection in a subject in need thereof, the infection is a mycobacterial infection. In some embodiments of a method of treating or preventing an infection in a subject in need thereof, the infection is a mycoplasma infection. In some embodiments of a method of treating or preventing an infection in a subject in need thereof, the infection is a viral infection. In some embodiments of a method of treating or preventing an infection in a subject in need thereof, the infection is caused by a protozoan microorganism. In some embodiments of a method of treating or preventing an infection in a subject in need thereof, the infection is an eye infection. In some embodiments of a method of treating or preventing an infection in a subject in need thereof, the infection is associated with conjunctivitis, keratitis, blepharitis, meibomianitis, or endophthalmitis. In some embodiments of a method of treating or preventing an infection in a subject in need thereof, the infection is associated with acanthamoebic keratitis.

### Keratitis

Keratitis is a condition in which the eye's cornea, the front part of the eye, becomes inflamed. The condition is often marked by moderate to intense pain and usually involves any of the following symptoms: pain, impaired eyesight, photophobia, red eye, and a “gritty” sensation. In some embodiments, the keratitis is acute. In some embodiments, the keratitis is acute epithelial keratitis. In some embodiments, the keratitis is nummular keratitis. In some embodiments, the keratitis is interstitial keratitis. In some embodiments, the keratitis is disciform keratitis. In some embodiments, the keratitis is chronic. In some embodiments, the keratitis is neurotrophic keratitis. In some embodiments, the keratitis is mucous plaque keratitis. In some embodiments, the keratitis is caused by an infection. In some embodiments, the keratitis is viral. In some embodiments, the keratitis is herpes simplex keratitis (dendritic keratitis—viral infection of the cornea is often caused by the herpes simplex virus which frequently leaves what is called a ‘dendritic ulcer’). In some embodiments, the keratitis is herpes zoster keratitis, associated with Herpes zoster ophthalmicus, which is a form of shingles. In some embodiments, the keratitis is bacterial keratitis. Bacterial infection of the cornea can follow from an injury or from wearing contact lenses. The bacteria involved are Staphylococcus aureus and for contact lens wearers, Pseudomonas aeruginosa. Pseudomonas aeruginosa contains enzymes that can digest the cornea. In some embodiments, the keratitis is fungal keratitis. In some embodiments, the keratitis is amoebic. In some embodiments, the keratitis is acanthamoebic keratitis. Amoebic infection of the cornea is a serious corneal infection, often affecting contact lens wearers. It is usually caused by Acanthamoeba. In some embodiments, the keratitis is parasitic. In some embodiments, the keratitis is onchocercal keratitis, which follows O. volvulus infection by infected blackfly bite. These blackfly usually dwell near fast-flowing African streams, so the disease is also called “river blindness”. In some embodiments, the keratitis is superficial punctate keratitis. In some embodiments, the keratitis is ulcerative keratitis. In some embodiments, the keratitis is exposure keratitis—due to dryness of the cornea caused by incomplete or inadequate eyelid closure. In some embodiments, the keratitis is photokeratitis—keratitis due to intense ultraviolet radiation exposure (e.g. snow blindness or welder's arc eye).

### Acanthamoeba Keratitis

Acanthamoeba keratitis is a painful, vision-threatening corneal infection caused by pathogenic environmental amoebae. Acanthamoeba spp. are free-living opportunistic protozoa pathogens that are ubiquitous in many diverse environments including air, soil, freshwater, seawater, tap water, bottled water, swimming pools, sewage, and vegetables. Corneal infection with Acanthamoeba spp. was first described in humans during the mid-1970's. Since this time, dramatic increases in the number of human cases have been reported and Acanthamoeba keratitis is now widely recognized as an emerging ocular surface infection.

The frequency of Acanthamoeba keratitis is continuing to increase in many countries, including the United States, as a result of an expanding population of individuals at risk for infection, enhanced recognition of infection, improvements in diagnostic techniques, and sporadic outbreaks of infection associated with alterations of contact lens wear, contact lens handling, and water quality. The true incidence of Acanthamoeba keratitis is not currently known and varies widely among different geographical regions; however, recent estimates include 1 case per 30,000 hydrogel contact lens wearers per year in Europe, England, and Hong Kong; 1 case per 10,000 contact lens wearers per year in the United States; and 1 case per 4,200 outpatient visits to tertiary referral hospitals in Australia.

Acanthamoeba keratitis most commonly occurs in immunocompetent, healthy individuals and the most important and prevalent risk factors are contact lens wear, corneal trauma, and exposure to contaminated water or soil. Acanthamoeba trophozoites and cysts are capable of firmly adhering to soft contact lenses, a characteristic that explains the strong association between contact lens wear and infection. The clinical features of Acanthamoeba keratitis are diverse and non-specific. Corneal epithelial irregularities, epithelial ulcers, stromal infiltrates, stromal ulcers, radial keratoneuritis, and keratomalacia are common. These clinical features mimic infection with bacteria, viruses, and fungi; and amoebic keratitis is often initially misdiagnosed clinically. The diagnosis of Acanthamoeba keratitis is based upon demonstration of the organism or its nucleic acid with culture, cytology, histopathology, PCR, or in vivo corneal confocal microscopy.

Treatment of Acanthamoeba keratitis is lengthy and difficult, as few agents are effective at eliminating the cyst stage of Acanthamoeba within corneal tissues. Various therapeutic agents, including the diamidines, biguanides, imidazoles, and aminoglycosides have been reported with variable treatment results. Adverse ocular reactions attributable to the toxic properties of these compounds occur frequently. Treatment is often continued for a year or more and infection recurrences may develop after apparently successful medical therapy. Therapeutic keratoplasty has been used to manage acute complications associated with Acanthamoeba keratitis, but with generally poor results attributable to graft rejection and extension of Acanthamoeba infection into the graft. Therapeutic keratoplasty to resolve corneal scarring and astigmatism performed following a course of medical anti-amoebic therapy is associated with an improved surgical prognosis; however, exacerbation of ocular inflammation and graft infection still occur frequently.

The principal challenge for improving outcomes of patients with Acanthamoeba keratitis is the development of more effective drugs for the treatment and elimination of Acanthamoeba cysts within corneal tissues. Additionally, the pathogenesis of Acanthamoeba keratitis remains incompletely understood and the relative contribution of Acanthamoeba and host-derived factors in inducing tissue destruction is not clear. A major obstacle to improved understanding and treatment of Acanthamoeba keratitis is the lack of an appropriate animal model for comparative study. Acanthamoeba keratitis has been experimentally induced in pigs, rats, and Chinese hamsters; however, no spontaneous amoeba corneal infections have been identified in these animal species and the models have only been used on a limited basis since their publication. These animal models of Acanthamoeba keratitis develop ocular lesions that are clinically dissimilar from that of humans, require extensive host manipulation to induce infection, and possess ocular anatomy and physiology that is markedly different than humans.

### Conjunctivitis

Conjunctivitis, also known as pink eye, is inflammation of the outermost layer of the white part of the eye and the inner surface of the eyelid. There may also be pain, burning, scratchiness, or itchiness. The affected eye may have increased tears or be “stuck shut” in the morning. Swelling of the white part of the eye may also occur. Itching of the eye is more common in cases due to allergies. Conjunctivitis can affect one or both eyes. The most common infectious causes are viral followed by bacterial. The viral infection may occur along with other symptoms of a common cold. Viral and bacterial cases are easily spread between people. Allergies to pollen or animal hair are also common causes. In some embodiments, the conjunctivitis is viral conjunctivitis. In some embodiments, the conjunctivitis is bacterial conjunctivitis. In some embodiments, the conjunctivitis is allergic conjunctivitis. In some embodiments, the conjunctivitis is chemical conjunctivitis.

### Viral Conjunctivitis

Viral conjunctivitis is often associated with an infection of the upper respiratory tract, a common cold, and/or a sore throat. Its symptoms include excessive watering and itching of the eye. The infection usually begins with one eye, but may spread easily to the other. Viral conjunctivitis shows a fine, diffuse pinkness of the conjunctiva, which is easily mistaken for the ciliary infection of iris (iritis), but there are usually corroborative signs on microscopy, particularly numerous lymphoid follicles on the tarsal conjunctiva, and sometimes a punctate keratitis. Some other viruses that can infect the eye include Herpes simplex virus and Varicella zoster.

### Allergic Conjunctivitis

Allergic conjunctivitis is inflammation of the conjunctiva (the membrane covering the white part of the eye) due to allergy. Allergens differ among patients. Symptoms consist of redness (mainly due to vasodilation of the peripheral small blood vessels), swelling of the conjunctiva, itching, and increased lacrimation (production of tears). If this is combined with rhinitis, the condition is termed “allergic rhinoconjunctivitis”. The symptoms are due to release of histamine and other active substances by mast cells, which stimulate dilation of blood vessels, irritate nerve endings, and increase secretion of tears.

### Bacterial Conjunctivitis

Bacterial conjunctivitis causes the rapid onset of conjunctival redness, swelling of the eyelid, and mucopurulent discharge. Typically, symptoms develop first in one eye, but may spread to the other eye within 2-5 days. Bacterial conjunctivitis due to common pyogenic (pus-producing) bacteria causes marked grittiness/irritation and a stringy, opaque, greyish or yellowish mucopurulent discharge that may cause the lids to stick together, especially after sleep. Severe crusting of the infected eye and the surrounding skin may also occur. The gritty and/or scratchy feeling is sometimes localized enough for patients to insist they must have a foreign body in the eye. The more acute pyogenic infections can be painful. Common bacteria responsible for non-acute bacterial conjunctivitis are Staphylococci, Streptococci, Haemophilus sp. Less commonly Chlamydia trachomatis is involved. Bacteria such as Chlamydia trachomatis or Moraxella can cause a non-exudative but persistent conjunctivitis without much redness. Bacterial conjunctivitis may cause the production of membranes or pseudomembranes that cover the conjunctiva. Pseudomembranes consist of a combination of inflammatory cells and exudates, and are loosely adherent to the conjunctiva, while true membranes are more tightly adherent and cannot be easily peeled away. Cases of bacterial conjunctivitis that involve the production of membranes or pseudomembranes are associated with Neisseria gonorrhoeae, β-hemolytic streptococci, and C. diphtheriae.

### Chemical Conjunctivitis

Chemical eye injury is due to either an acidic or alkali substance getting in the eye Alkali burns are typically worse than acidic burns. Mild burns will produce conjunctivitis, while more severe burns may cause the cornea to turn white. Litmus paper is an easy way to rule out the diagnosis by verifying that the pH is within the normal range of 7.0-7.2. Large volumeirrigation is the treatment of choice and should continue until the pH is 6-8. Local anaesthetic eye drops can be used to decrease the pain. Irritant or toxic conjunctivitis show primarily marked redness. If due to splash injury, it is often present in only the lower conjunctival sac. With some chemicals, above all with caustic alkalis such as sodium hydroxide, there may be necrosis of the conjunctiva with a deceptively white eye due to vascular closure, followed by sloughing of the dead epithelium. This is likely to be associated with slit-lamp evidence of anterior uveitis.

### Blepharitis

Blepharitis is a common eye condition characterized by inflammation of the eyelid, resulting in inflamed, irritated, itchy, and reddened eyelids (characterized by a scaly crust on the lid margins). A number of diseases and conditions can lead to blepharitis. It can be caused by the oil glands at the base of the eyelashes becoming clogged, a bacterial infection, allergies, or other conditions. Often blepharitis is a long-standing chronic inflammation of varying severity. In some embodiments, the blepharitis is seborrhoeic blepharitis. In some embodiments, the blepharitis is staphylococcal blepharitis. In some embodiments, the blepharitis is anterior blepharitis. In some embodiments, the blepharitis is posterior blepharitis. In some embodiments, the blepharitis is parasitic blepharitis.

Blepharitis is typically caused by bacterial infection or blockage of the eyelid's oil glands, although sometimes it is caused by allergies. Various diseases and conditions can lead to blepharitis, such as rosacea, herpes simplex dermatitis, varicella-zoster dermatitis, molluscum contagiosum, allergic dermatitis, contact dermatitis, seborrheic dermatitis, staphylococcal dermatitis, and parasitic infections (e.g., Demodex and Phthiriasis palpebrarum).

The parasite, Demodex folliculorum (D. folliculorum), causes Blepharitis when present in excessive numbers within the dermis of the eye lids and can live for approximately 15 days. The parasite's adult and egg forms live on the hair follicle, inhabiting the sebaceous and aprocrine gland of the human lid. Direct contact allows this pathogen to spread. Factors that allow this pathogen to multiply include hypervascular tissue, poor hygienic conditions, and immune deficiency. In treating Blepharitis caused by D. folliculorum, mechanical cleaning and proper hygiene is important towards decreasing the parasite's numbers.

Blepharitis usually does not cause permanent eyesight damage. Chronic blepharitis may result in damage of varying severity which may have a negative effect upon vision and therefore upon the eyeglass prescription. Long-term untreated blepharitis can lead to eyelid scarring, excess tearing, difficulty wearing contact lenses, development of an internal hordeolum, a stye (external hordeolum: an infection near the base of the eyelashes, resulting in a painful lump on the edge of the eyelid), or a chalazion (a blockage/bacteria infection in a small oil glands at the margin of the eyelid, just behind the eyelashes, leading to a red, swollen eyelid), chronic pink eye (conjunctivitis), keratitis, and cornea ulcer or irritation. The lids may become red and may have ulcerative, non-healing areas which may bleed.

Blepharitis can cause blurred vision due to a poor tear film Also, the tears might seem frothy or bubbly in nature and mild scarring might occur to the eyelids. The symptoms and signs of blepharitis are often erroneously ascribed by the patient as being due to “recurrent conjunctivitis”.

Staphylococcal blepharitis

Staphylococcal blepharitis is caused by infection of the anterior portion of the eyelid by Staphylococcal bacteria. Symptoms include a foreign body sensation, matting of the lashes, and burning. Collarette around eyelashes, a ring-like formation around the lash shaft, can be observed. Other symptoms include loss of eyelashes or broken eyelashes. The condition can sometimes lead to a chalazion or a stye.

Staphylococcal blepharitis is a condition which may start in childhood and continue through adulthood. It is commonly recurrent and it requires special medical care. The prevalence of Staphylococcus aureus in the conjunctival sac and on the lid margin varies among countries, probably due to climate. According to various studies, Staphylococcal blepharitis occurs more commonly in women than men and occurs more frequently in warmer climates. Chronic bacterial blepharitis may lead to ectropion.

### Posterior Blepharitis or Rosacea-Associated Blepharitis

Posterior blepharitis is inflammation of the eyelids secondary to dysfunction of the meibomian glands. Like anterior blepharitis, it is a bilateral chronic condition and is manifested by a broad spectrum of symptoms involving the lids including inflammation and plugging of the meibomian orifices and production of abnormal secretion upon pressure over the glands. It may be associated with skin rosacea, and there is growing evidence that in some cases it is caused by demodex mites.

### Endophthalmitis

Endophthalmitis is an intraocular infection that commonly occurs after cataract surgery or ocular trauma. The causative agent in post-operative endopthalmitis is typically a bacteria, often the causative bacteria is Staphylococcus Epidermidis. In some cases of endogenous endophthalmitis—particularly in immunocompromised patients or those with diabetes—the spread of infection may have been hematogenous (via the blood-stream).

Endophthalmitis is usually accompanied by severe pain, loss of vision, and redness of the conjunctiva and the underlying episclera. Hypopyon can be present in endophthalmitis and should be looked for on examination by a slit lamp.

An eye exam may be indicated in severe forms of candidiasis. 1-3% of cases of candidal blood infections include endophthalmitis.

In some embodiments, the cause of endophthalmitis is bacterial (N. meningitidis, Staphylococcus aureus, S. epidermidis, S. pneumoniae, other streptococcal spp., Propionibacterium acnes, Pseudomonas aeruginosa, other gram negative organisms). In some embodiments, the cause of endophthalmitis is viral (Herpes simplex virus). In some embodiments, the cause of endophthalmitis is fungal (Candida spp. Fusarium). In some embodiments, the cause of endophthalmitis is parasitic (Toxoplasma gondii, Toxocara).

### Meibomianitis

Meibomitis or Meibomianitis is an inflammatory condition of the Meibomian glands which is often seen in patients with acne or rosacea. Along the margin of the lids there are a series of small sebaceous glands called the meibomian glands. The meibomian glands create and distribute an oily substance called lipids. Whenever the eye blinks the produced oil coat the tear layer protecting it from evaporation. When a patient is affected by a inflammation the produced oil is less in quantity and worse in quality as a result quicker tear evaporation. In addition, these oils become stagnant and bacteria, usually staph bacteria, colonize inside the meiboinian glands. Moreover, these bacteria produce toxins that cause the lids to become red and inflamed. The symptoms are different for each patient but most of the times are similar to common blepharitis.

### Uveitis

Uveitis is the inflammation of the uvea, the pigmented layer that lies between the inner retina and the outer fibrous layer composed of the sclera and cornea. The uvea consists of the middle layer of pigmented vascular structures of the eye and includes the iris, ciliary body, and choroid. Uveitis is an ophthalmic emergency and requires a thorough examination by an optometrist or ophthalmologist and urgent treatment to control the inflammation.

Uveitis is classified anatomically into anterior, intermediate, posterior, and panuveitic forms—based on the part of the eye primarily affected.

Anterior uveitis includes iridocyclitis and iritis. Iritis is the inflammation of the anterior chamber and iris. Iridocyclitis presents the same symptoms as iritis, but also includes inflammation in the ciliary body. Anywhere from two-thirds to 90% of uveitis cases are anterior in location. This condition can occur as a single episode and subside with proper treatment or may take on a recurrent or chronic nature. Intermediate uveitis, also known as pars planitis, consists of vitritis—which is inflammation of cells in the vitreous cavity, sometimes with snowbanking, or deposition of inflammatory material on the pars plana. There are also “snowballs,” which are inflammatory cells in the vitreous. Posterior uveitis or chorioretinitis is the inflammation of the retina and choroid. Pan-uveitis is the inflammation of all layers of the uvea.

Also disclosed herein are methods of preventing an eye infection in a subject in need thereof prior to an eye surgery, the method comprising administering to the subject in need thereof a pharmaceutical composition described herein. In some embodiments of a method of preventing an eye infection in a subject in need thereof prior to an eye surgery, the pharmaceutical composition comprises (a) a chlorite salt; (b) a quaternary ammonium salt; and (c) ammonium chloride. In some embodiments of a method of preventing an eye infection in a subject in need thereof prior to an eye surgery, the pharmaceutical composition comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments of a method of preventing an eye infection in a subject in need thereof prior to an eye surgery, the pharmaceutical composition comprises (a) about 0.0003% (w/w) of a chlorite salt; (b) about 0.001% (w/w) of a quaternary ammonium salt; and (c) about 0.2% (w/w) of ammonium chloride. In some embodiments of a method of preventing an eye infection in a subject in need thereof prior to an eye surgery, the pharmaceutical composition comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments of a method of preventing an eye infection in a subject in need thereof prior to an eye surgery, the pharmaceutical composition comprises (a) about 0.0003% (w/w) of sodium chlorite; (b) about 0.001% (w/w) of a quaternary ammonium salt; and (c) about 0.2% (w/w) of ammonium chloride. In some embodiments of a method of preventing an eye infection in a subject in need thereof prior to an eye surgery, the pharmaceutical composition comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments of method of preventing an eye infection in a subject in need thereof prior to an eye surgery, the pharmaceutical composition comprises (a) about 0.01% (w/w) of stabilized chlorine dioxide; (b) about 0.001% (w/w) of a quaternary ammonium salt; and (c) about 0.2% (w/w) of ammonium chloride. In some embodiments of method of preventing an eye infection in a subject in need thereof prior to an eye surgery, the pharmaceutical composition comprises (a) about 0.02% (w/w) of stabilized chlorine dioxide; (b) about 0.001% (w/w) of a quaternary ammonium salt; and (c) about 0.025% (w/w) of ammonium chloride. In some embodiments of method of preventing an eye infection in a subject in need thereof prior to an eye surgery, the pharmaceutical composition comprises (a) about 0.03% (w/w) of stabilized chlorine dioxide; (b) about 0.003% (w/w) of a quaternary ammonium salt; and (c) about 0.025% (w/w) of ammonium chloride.

Also disclosed herein are methods of treating or preventing an eye infection in a subject in need thereof after an eye surgery, the method comprising administering to the subject in need thereof a pharmaceutical composition described herein. In some embodiments of a method of treating or preventing an eye infection in a subject in need thereof after an eye surgery, the pharmaceutical composition comprises (a) a chlorite salt; (b) a quaternary ammonium salt; and (c) ammonium chloride. In some embodiments of a method of treating or preventing an eye infection in a subject in need thereof after an eye surgery, the pharmaceutical composition comprises (a) about 0.0001% to about 1% (w/w) of a chlorite salt; (b) about 0.0001% to about 0.5% (w/w) of a quaternary ammonium salt; and (c) about 0.001% to about 2% (w/w) of ammonium chloride. In some embodiments of a method of treating or preventing an eye infection in a subject in need thereof after an eye surgery, the pharmaceutical composition comprises (a) about 0.0003% (w/w) of a chlorite salt; (b) about 0.001% (w/w) of a quaternary ammonium salt; and (c) about 0.2% (w/w) of ammonium chloride. In some embodiments of a method of treating or preventing an eye infection in a subject in need thereof after an eye surgery, the pharmaceutical composition comprises (a) about 0.0001% to about 1% (w/w) of sodium chlorite; (b) about 0.0001% to about 0.5% (w/w) of a quaternary ammonium salt; and (c) about 0.001% to about 2% (w/w) of ammonium chloride. In some embodiments of a method of treating or preventing an eye infection in a subject in need thereof after an eye surgery, the pharmaceutical composition comprises (a) about 0.0003% (w/w) of sodium chlorite; (b) about 0.001% (w/w) of a quaternary ammonium salt; and (c) about 0.2% (w/w) of ammonium chloride. In some embodiments of a method of treating or preventing an eye infection in a subject in need thereof after an eye surgery, the pharmaceutical composition comprises (a) about 0.005% to about 1% (w/w) of chlorine dioxide; (b) about 0.0001% to about 0.5% (w/w) of a quaternary ammonium salt; and (c) about 0.001% to about 2% (w/w) of ammonium chloride. In some embodiments of method of treating or preventing an eye infection in a subject in need thereof after an eye surgery, the pharmaceutical composition comprises (a) about 0.01% (w/w) of stabilized chlorine dioxide; (b) about 0.001% (w/w) of a quaternary ammonium salt; and (c) about 0.2% (w/w) of ammonium chloride. In some embodiments of method of treating or preventing an eye infection in a subject in need thereof after an eye surgery, the pharmaceutical composition comprises (a) about 0.02% (w/w) of stabilized chlorine dioxide; (b) about 0.001% (w/w) of a quaternary ammonium salt; and (c) about 0.025% (w/w) of ammonium chloride. In some embodiments of method of treating or preventing an eye infection in a subject in need thereof after an eye surgery, the pharmaceutical composition comprises (a) about 0.03% (w/w) of stabilized chlorine dioxide; (b) about 0.003% (w/w) of a quaternary ammonium salt; and (c) about 0.025% (w/w) of ammonium chloride. In some embodiments, the eye surgery is laser eye surgery, cataract surgery, glaucoma surgery, refractive surgery, corneal surgery, vitreo-retinal surgery, eye muscle surgery, oculoplastic surgery, surgery involving the lacrimal apparatus, or eye removal.

### Laser Eye Surgery

Although the terms laser eye surgery and refractive surgery are commonly used as if they were interchangeable, this is not the case. Lasers may be used to treat nonrefractive conditions (e.g. to seal a retinal tear). Laser eye surgery or laser corneal surgery is a medical procedure that uses a laser to reshape the surface of the eye. This is done to correct myopia (short-sightedness), hypermetropia (long sightedness) and astigmatism (uneven curvature of the eye's surface). It is important to note that refractive surgery is not compatible with everyone, and rarely people may find that eyewear is still needed after surgery.

### Cataract Surgery

A cataract is an opacification or cloudiness of the eye's crystalline lens due to aging, disease, or trauma that typically prevents light from forming a clear image on the retina. If visual loss is significant, surgical removal of the lens may be warranted, with lost optical power usually replaced with a plastic intraocular lens (IOL). Owing to the high prevalence of cataracts, cataract extraction is the most common eye surgery.

### Glaucoma Surgery

Glaucoma is a group of diseases affecting the optic nerve that results in vision loss and is frequently characterized by raised intraocular pressure (TOP). There are many types of glaucoma surgery, and variations or combinations of those types, that facilitate the escape of excess aqueous humor from the eye to lower intraocular pressure, and a few that lower TOP by decreasing the production of aqueous humor.

### Canaloplasty

Canaloplasty is an advanced, nonpenetrating procedure designed to enhance drainage through the eye's natural drainage system to provide sustained reduction of TOP. Canaloplasty utilizes microcatheter technology in a simple and minimally invasive procedure. To perform a canaloplasty, an Ophthalmologist creates a tiny incision to gain access to a canal in the eye. A microcatheter circumnavigates the canal around the iris, enlarging the main drainage channel and its smaller collector channels through the injection of a sterile, gel-like material called viscoelastic. The catheter is then removed and a suture is placed within the canal and tightened. By opening up the canal, the pressure inside the eye can be reduced.

### Refractive Surgery

Refractive surgery aims to correct errors of refraction in the eye, reducing or eliminating the need for corrective lenses.

### Corneal Surgery

Corneal surgery includes most refractive surgery as well as the following:
Corneal transplant surgery used to remove a cloudy/diseased cornea and replace it with a clear donor cornea.
Penetrating keratoplasty (PK)
Keratoprosthesis(KPro)
Phototherapeutic keratectomy (PTK)
Pterygium excision
Corneal tattooing
Osteo-Odonto-Keratoprosthesis (OOKP), in which support for an artificial cornea is created from a tooth and its surrounding jawbone. This is a still-experimental procedure used for patients with severely damaged eyes, generally from burns.
Eye color change surgery through an iris implant, known as Brightocular, or stripping away the top layer of eye pigment, known as the Stroma procedure.

### Vitreo-Retinal Surgery

Vitreo-retinal surgery includes the following:
Vitrectomy: Anterior vitrectomy is the removal of the front portion of vitreous tissue. It is used for preventing or treating vitreous loss during cataract or corneal surgery, or to remove misplaced vitreous in conditions such as aphakia pupillary block glaucoma.
Pars plana vitrectomy (PPV), or trans pars plana vitrectomy (TPPV), is a procedure to remove vitreous opacities and membranes through a pars plana incision. It is frequently combined with other intraocular procedures for the treatment of giant retinal tears, tractional retinal detachments, and posterior vitreous detachments.
Pan retinal photocoagulation (PRP) is a type of photocoagulation therapy used in the treatment of diabetic retinopathy.
Retinal detachment repair Ignipuncture is an obsolete procedure that involves cauterization of the retina with a very hot pointed instrument.
A scleral buckle is used in the repair of a retinal detachment to indent or “buckle” the sclera inward, usually by sewing a piece of preserved sclera or silicone rubber to its surface.
Laser photocoagulation, or photocoagulation therapy, is the use of a laser to seal a retinal tear.
Pneumatic retinopexy
Retinal cryopexy, or retinal cryotherapy, is a procedure that uses intense cold to induce a chorioretinal scar and to destroy retinal or choroidal tissue.
Macular hole repair
Partial lamellar sclerouvectomy
Partial lamellar sclerocyclochoroidectomy
Partial lamellar sclerochoroidectomy
Posterior sclerotomy is an opening made into the vitreous through the sclera, as for detached retina or the removal of a foreign body
Radial optic neurotomy
macular translocation surgery through 360 degree retinotomy or through scleral imbrication technique

### Oculoplastic Surgery

Oculoplastic surgery, or oculoplastics, is the subspecialty of ophthalmology that deals with the reconstruction of the eye and associated structures. Oculoplastic surgeons perform procedures such as the repair of droopy eyelids (blepharoplasty), repair of tear duct obstructions, orbital fracture repairs, removal of tumors in and around the eyes, and facial rejuvenation procedures including laser skin resurfacing, eye lifts, brow lifts, and even facelifts.

### Eye Removal

An enucleation is the removal of the eye leaving the eye muscles and remaining orbital contents intact. An evisceration is the removal of the eye's contents, leaving the scleral shell intact. Usually performed to reduce pain in a blind eye. An exenteration is the removal of the entire orbital contents, including the eye, extraocular muscles, fat, and connective tissues; usually for malignant orbital tumors.

### Surgery Involving the Lacrimal Apparatus

Surgery involving the lacrimal apparatus includes the following:
A dacryocystorhinostomy (DCR) or dacryocystorhinotomy is a procedure to restore the flow of tears into the nose from the lacrimal sac when the nasolacrimal duct does not function.
Canaliculodacryocystostomy is a surgical correction for a congenitally blocked tear duct in which the closed segment is excised and the open end is joined to the lacrimal sac.
Canaliculotomy involves slitting of the lacrimal punctum and canaliculus for the relief of epiphora
A dacryoadenectomy is the surgical removal of a lacrimal gland.
A dacryocystectomy is the surgical removal of a part of the lacrimal sac.
A dacryocystostomy is an incision into the lacrimal sac, usually to promote drainage.
A dacryocystotomy is an incision into the lacrimal sac.

### Combinations

Also described herein are pharmaceutical compositions further comprising an additional therapeutic agent. In some embodiments, the additional therapeutic agent is an anesthetic agent. In some embodiments, the anesthetic agent is selected from lidocaine, proparacaine, tetracaine, proparacaine, proparacaine, and any combinations thereof. In some embodiments, the anesthetic agent is lidocaine.

In some embodiments, the additional therapeutic agent is an antiseptic. In some embodiments, the additional therapeutic agent is Povidone-iodine. In some embodiments, the additional therapeutic agent is chlorhexidine.

In some embodiments, the additional therapeutic agent is an antibacterial agent. In some embodiments, the additional therapeutic agent is moxifloxacin, ciprofloxacin, besifloxacin, gatifloxacin, erythromycin, or any combinations thereof.

In some embodiments, the additional therapeutic agent is an antiviral agent. some embodiments, the additional therapeutic agent is gangcyclovir, trifluridine, or any combinations thereof.

In some embodiments, the additional therapeutic agent is an antifungal agent. In some embodiments, the additional therapeutic agent is natamycin, amphotericin B, or any combinations thereof.

### EXAMPLES

### Example 1

### Comparison of SOL01 and SOL02 Against Three Commercially-Available MPDS

Standard methods for assessing the antimicrobial activity and the cytotoxicity of MPDS have been harmonized under International Standards Organization (ISO) standard laboratory procedures using a battery of preselected bacterial and fungal species. The standards for testing provide a standardized comparison among multiple MPDS. The use of standard laboratory procedures for testing provides an opportunity for benchmarking against a common battery of microbial challenge species.

Two presently-disclosed MPDS designated SOL01 (also referred to as “SL1”) and SOL02 (also referred to as “SL2”) were developed to be broadly effective against multiple microbial pathogens by inclusion of multiple chemical stressors rather than a single disinfection agent while simultaneously exerting minimal cytotoxicity upon tissues upon contact. The compositions of SOL01 and SOL02 are shown in the table 1.

TABLE 1

| Ingredient % (w/w) | SOL01 (SL1) | SOL02 (SL2) |
|---|---|---|
| Stabilized chlorine dioxide* | 0.01 | 0.01 |
| C12-C14 | 0.002 | 0.002 |
| alkyl(ethylbenzyl)dimethylammonium | | |
| chloride | | |
| ammonium chloride | 0.005 | 0.005 |
| peracetic acid | 0 | 0.0005 |

*Stabilized chloride dioxide comprises about 3% sodium chlorite

The cytotoxicity and antimicrobial activity of SOL01 and SOL02 were compared against three commercially-available MPDS: ReNu fresh (active ingredient: Biguanide (0.0001%), Bausch & Lomb, Rochester, N.Y.), Opti-Free (active ingredients: Biguanide and Polyquad (polyquaternium; 0.001%), Alcon, Fort Worth, Tex.), and BioTrue (active ingredients: Biguanide (0.00013%), polyquaternium (0.0001%), and Hyaluronan (hyaluronic acid), Bausch & Lomb).

Relative antimicrobial disinfection and cytotoxicity of SOL01 and SOL02 and the three commercially-available MPDS were determined using ISO testing protocols 14729 (antimicrobial) and 10993 (cytotoxicity). The ISO-required battery of microorganisms (S. aureus, P. aeruginosa, S. marcesens, C. albicans, and F. solani) was used to assess the disinfection potential of each MPDS with contact times of one hour and four hours. Vero76 monkey kidney cells and XTT proliferation assay were used to assess in vitro cytotoxicity of each MPDS.

The disinfection potential of SOL01 and SOL02 in the ISO-specific antimicrobial assay demonstrated no bacterial or fungal plate growth for any of the microorganisms tested with as little as one hour contact time. All comparator MPDS, in contrast, showed significant growth at one hour contact times, with only slight reductions of bacterial or fungal growth after the four hours maximum contact time. Two of the three comparator MPDS exceeded 90% cell death at about 25% solution concentration dilution but required about 12% solution concentration dilution in order to minimize cytotoxicity from contact. SOL01 showed minimal cell death at approximately the same dilutions as comparator solutions, whereas SOL02 achieved similar performance with lower concentrations of about 1% to 3%.

The presently-disclosed MPDS (SOL01 and SOL02) combine broad spectrum antimicrobial killing against an array of microorganisms while maintaining minimal cytotoxicity on target tissues as demonstrated in vitro. The performance of SOL01 in the two ISO-recommended standard assays exceeded the three commercially-available comparator MPDS in antimicrobial activity, yet maintained a similar cytotoxicity profile. Optimization of SOL01 and SOL02 for maintaining hygiene of contact lenses and contact lens cases may reduce toxic effects while still maintaining effective prevention of ocular infections from microbial contamination.

### Cytotoxicity

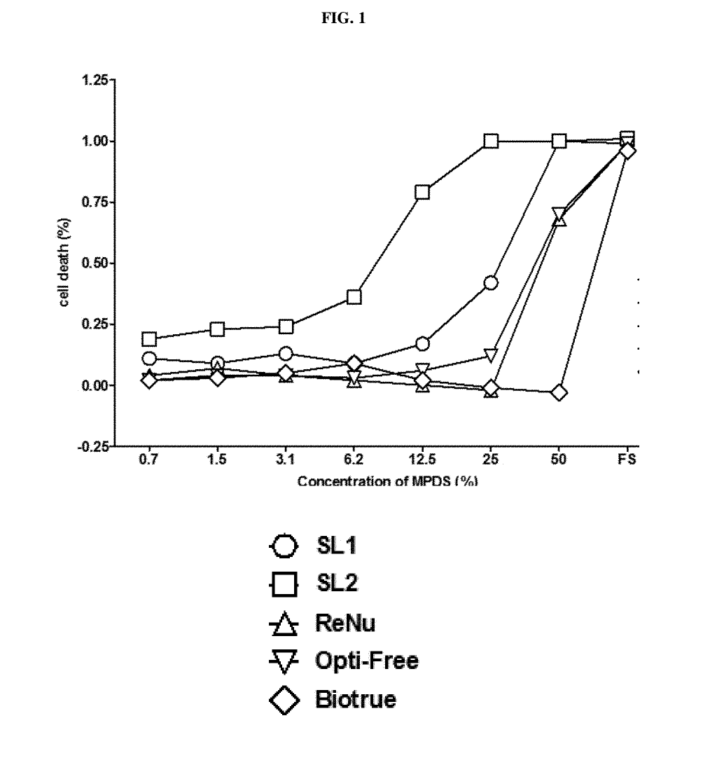

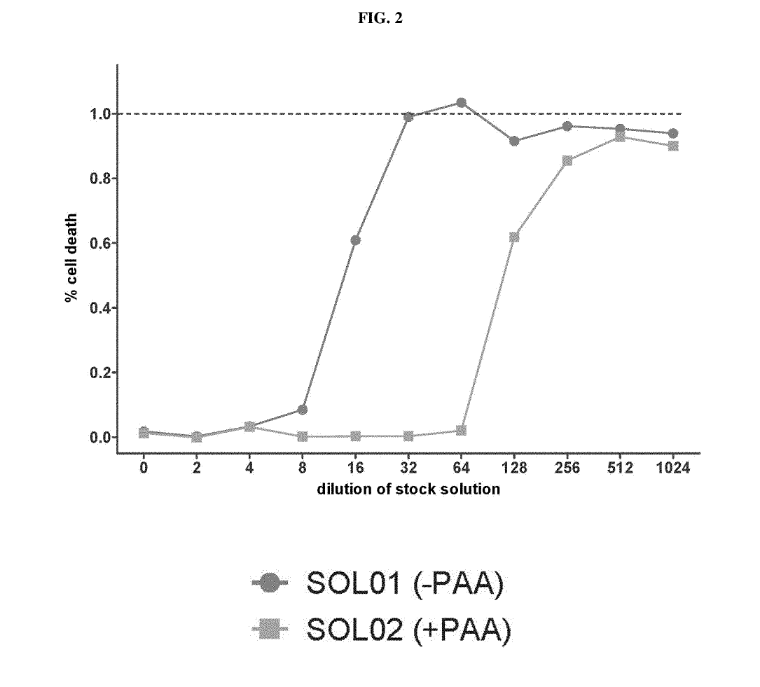

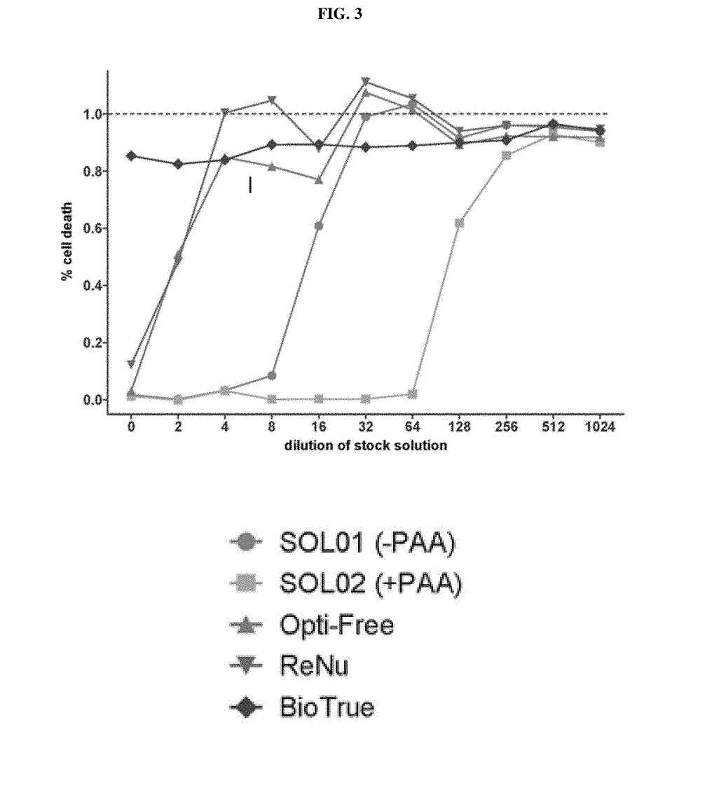

Cytotoxicity activity was evaluated in vitro based upon methods described in ISO standard 10993-5 (Biological Evaluation of Medical Devices—Part 5: Tests for in vitro cytotoxicity). A colony-formation assay was conducted using African Green Monkey kidney cells (Vero76), one cell line recommended for use under this testing regime. Approximately 1.0E+06 Vero76 cells in 2 mL of medium (minimum essential medium+5% fetal calf serum: Life Technologies, CA, USA) were inoculated into 6-well culture plates (Life Technologies, CA, USA) and cultured for about 48 hours at 37° C. at 5% CO₂ until adherent growth was confirmed. After washing and removal of the media, 1 mL of each SOL01, SOL02, and commercial MPDS was diluted with deionized sterile water and then added individually into individual wells at serial dilutions up to 1:512. Physiological saline was used as a negative control. Plates were incubated for about 48 hours at 37° C. at 5% CO₂ and then washed. Substrate was added and read at 490 nm. Proliferation was calculated in triplicate as (mean absorbance)×100/(mean absorbance/negative control group). As shown in FIG. 1, FIG. 2 and FIG. 3, results were plotted, and the relative cytotoxicity of each product compared.

All solutions were cytotoxic at full strength and showed a concentration-dependent increase in cytotoxicity as solution concentration increased. SOL02 was not cytotoxic at low concentrations of about 1% to about 3% but gradually increased to be highly cytotoxic at about 25%. ReNu, Opti-Free, and SOL01 were not cytotoxic at concentrations between about 25% and about 12.5% but increased two-fold in toxicity at about 50% concentration. BioTrue was not cytotoxic at about 50% solution concentration but was highly cytotoxic at full strength. Cytotoxicity rankings of the MPDS are as follows: BioTrue<Opti-Free≤ReNu≤SOL01<SOL02, as shown in FIG. 1.

### Antimicrobial Activity

Antimicrobial activity was evaluated by using the methods described as a ‘stand-alone’ contact lens disinfection test (ISO 14729, 2001), which requires that the MPDS product must be capable of reducing the viability of specified bacterial and fungal species by three logs (99.9%) and one log (90%), respectively, within a particular timeframe. The standard bacterial and fungal organisms prescribed by the standard method are Pseudomonas aeruginosa IF013275 (also referred to as “Pa”), Staphylococcus aureus IF013276 (also referred to as “Sa”), Serratia marcescens ATCC13880 (also referred to as “Sm”), Candida albicans IFO1594 (also referred to as “Ca”) and Fusarium solani ATCC36031 (also referred to as “Fs”). P. aeruginosa, S. aureus and S. marcescens were obtained commercially (ATCC, Manassas, Va.) as lyophilized packs. Bacterial cultures were reconstituted in soya broth and incubated for about 24 hours at 25° C. C. albicans and F. solani were obtained commercially (ATTC) and were grown in Sabouraud's dextrose in a similar fashion. All microorganisms were harvested using methods described in the ISO 14729 standard and adjusted for concentration through centrifugation and dilution with PBS. Final challenge concentrations of each approximated 1.0E+06 to 1.0E+07 CFU/ml.

A known volume (100 μl) of the microbial suspension was added to 9.9 mL of each MPDS in a polypropylene tube under sterile conditions. Mixtures were incubated at 25° C. for 1 hour, 2 hours, or 4 hours. After the prescribed contact time, the mixtures were sterile filtered through 0.45 μm analytical filter funnels (140-4045, Fisher Scientific) and washed twice with sterile DPBS. Each filter was aseptically removed from the filter funnel, laid upon one of TSA (P. aeruginosa, S. aureus), SDA (S. marcescens) or PDA (C. albicans and F. solani), and incubated at 25° C. for growth. Colonies were counted after about 48 hours of incubation. Each assay was performed in duplicate.

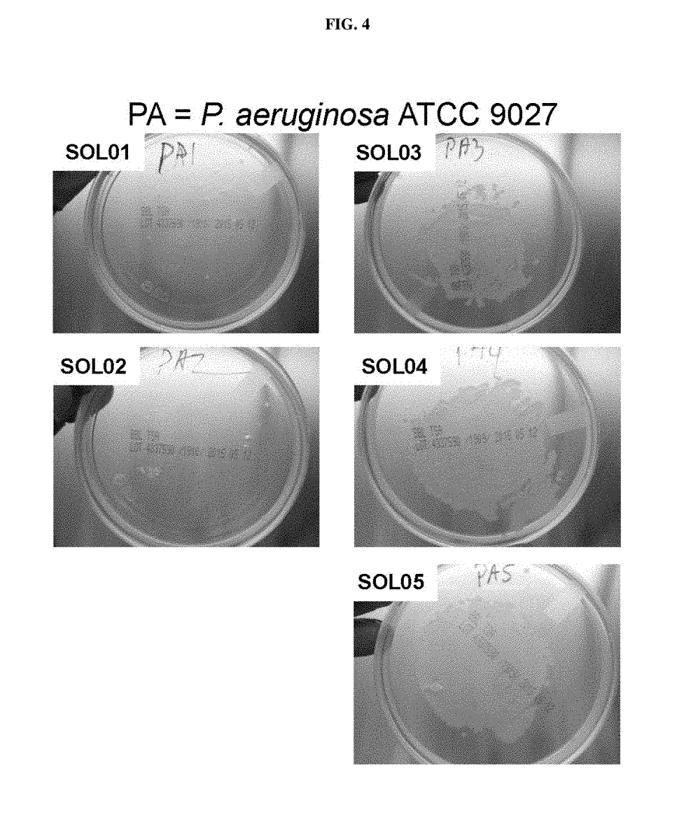

Both SOL01 and SOL02 were completely sterilizing against all of the microorganisms tested. As summarized in Table 2 and depicted in FIG. 4-8, no growth on any of the plates at either the one hour or four hour contact time point was observed.

TABLE 2

| | Contact | | | | | | | | | | | |
|---|---|---|---|---|---|---|---|---|---|---|---|---|
| MPDS | Time | | Pa | | Sa | | Sm | | Ca | | Fs | |
| | | Inoculating conc. | 4.5E+05 | | 1.03E+06 | | 1.0E+08 | | 7.9E+06 | | 3.9E+05 | |
| | 1 hr | (CFU/ml)² | CFU (log reduction) | | | | | | | | | |
| SL1 | | | 0 | (>5) | 0 | (>6) | 0 | (>8) | 0 | (>6) | 0 | (>5) |
| SL2 | | | 0 | (>5) | 0 | (>6) | 0 | (>8) | 0 | (>6) | 0 | (>5) |
| ReNu | | | TNTC | (0) | TNTC | (0) | 19 | (7) | TNTC | (0) | 134 | (4) |
| Opti-Free | | | TNTC | (0) | TNTC | (0) | TNTC | (0) | TNTC | (0) | 2 | (5) |
| BioTrue | | | TNTC | (0) | TNTC | (0) | 55 | (7) | 26 | (6) | 1 | (5) |
| | 4 hr | | CFU (log reduction) | | | | | | | | | |
| SL1 | | | 0 | (>5) | 0 | (>6) | 0 | (>8) | 0 | (>6) | 0 | (>5) |
| SL2 | | | 0 | (>5) | 0 | (>6) | 0 | (>8) | 0 | (>6) | 0 | (>5) |
| ReNu | | | TNTC | (0) | TNTC | (0) | 34 | (7) | 52 | (6) | 4 | (5) |
| Opti-Free | | | TNTC | (0) | TNTC | (0) | 79 | (7) | 164 | (5) | 0 | (>5) |
| BioTrue | | | TNTC | (0) | TNTC | (0) | 3 | (8) | 13 | (6) | 0 | (>5) |

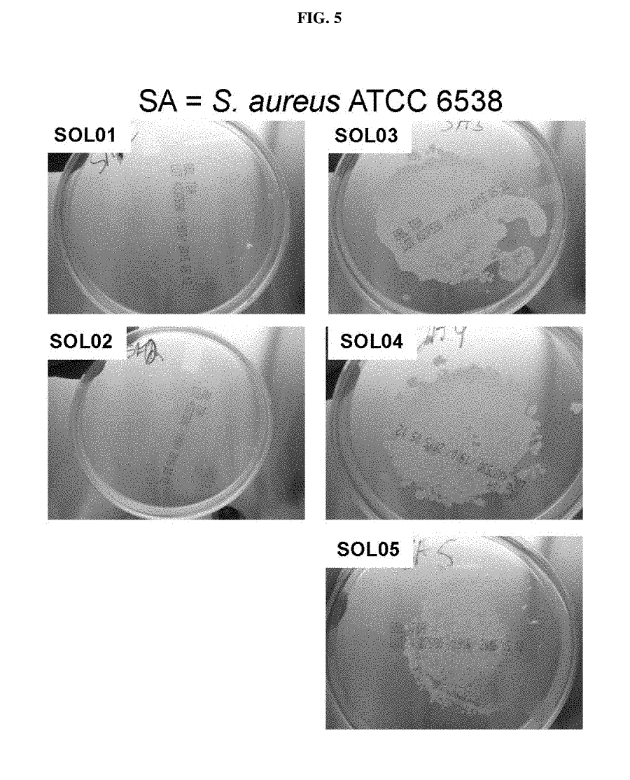

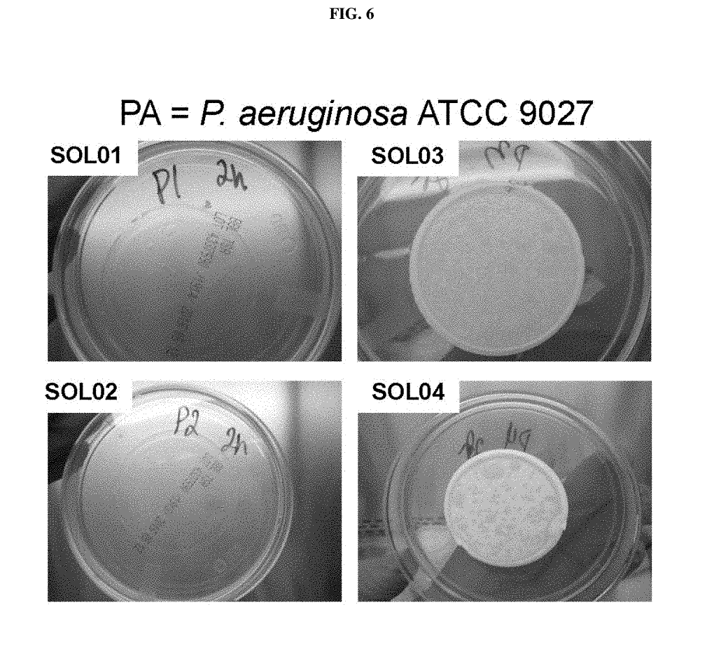

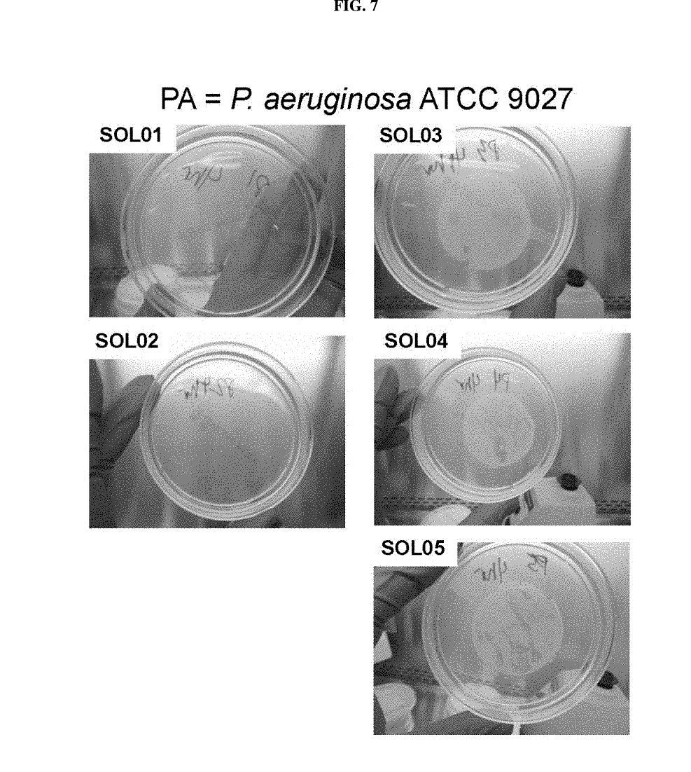

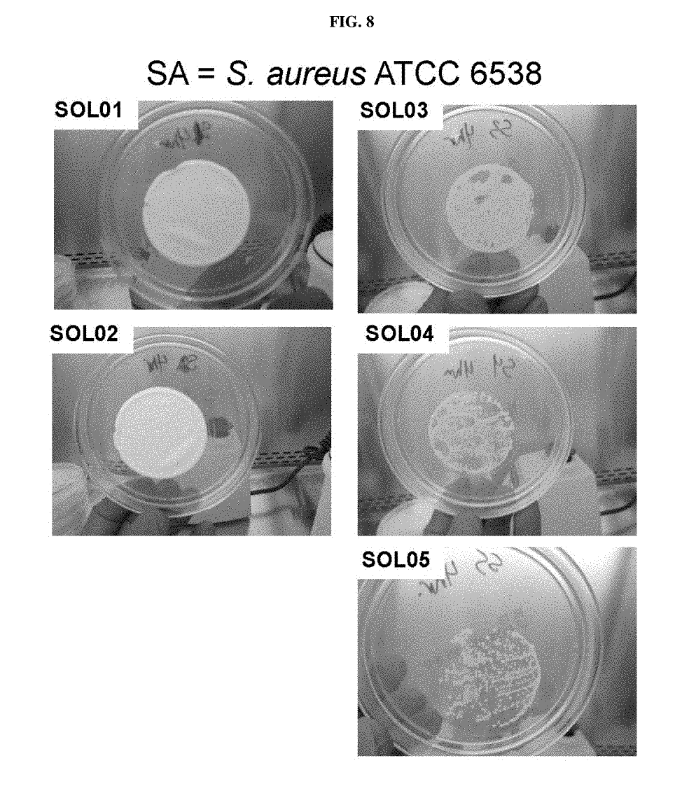

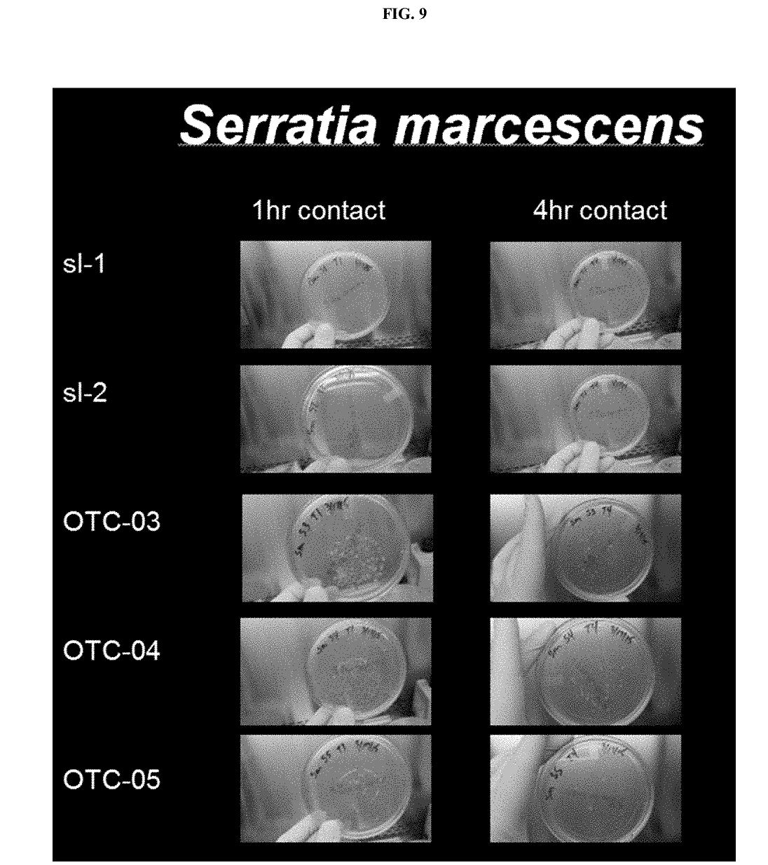

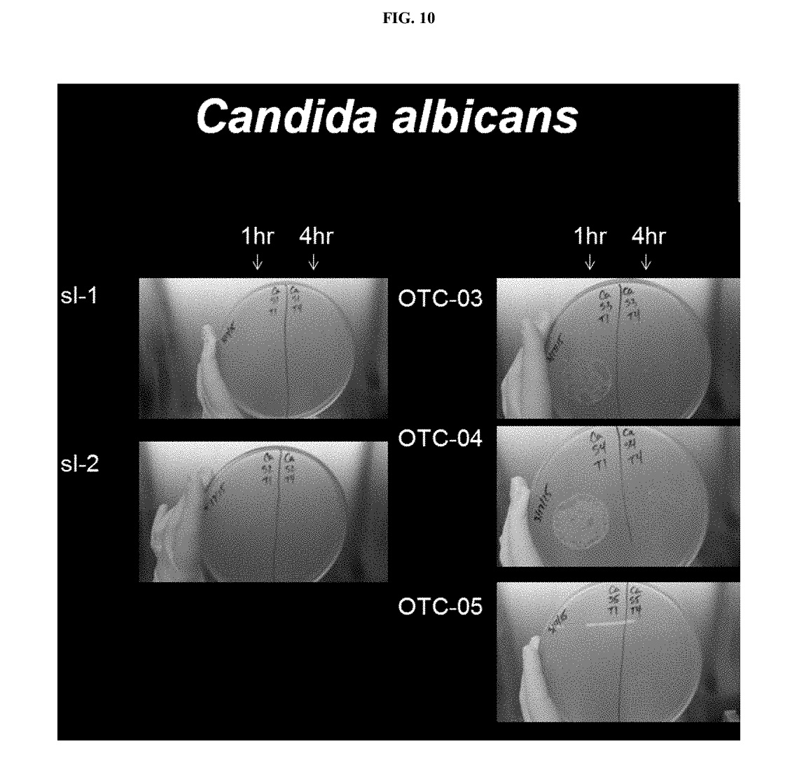

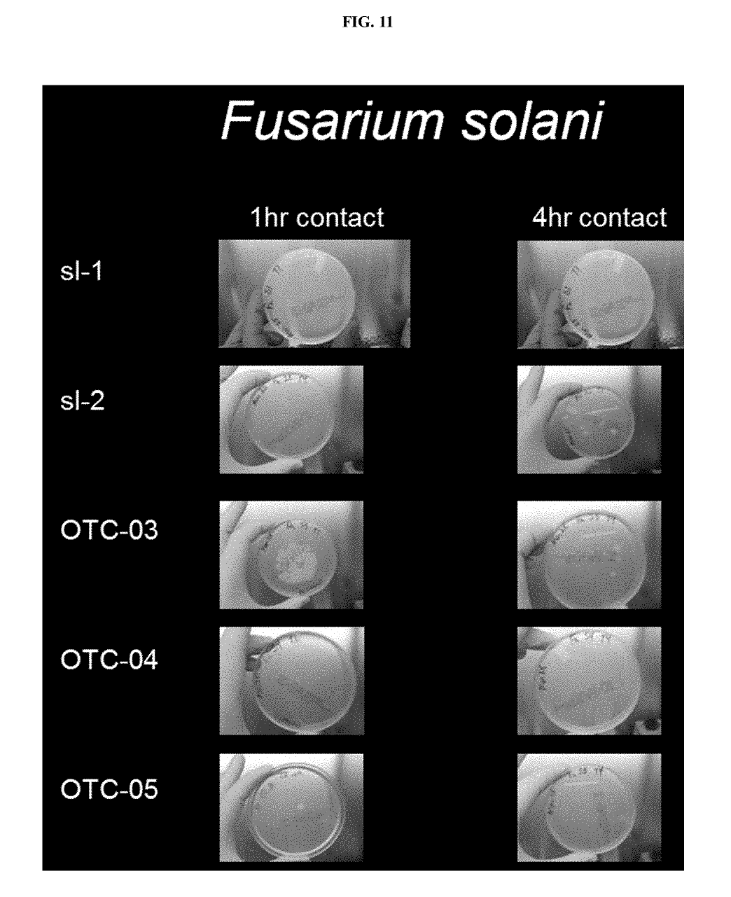

In contrast, none of the three commercially available MPDS tested were effective in achieving any log reduction against P. aeruginosa (see FIG. 4, FIG. 6 and FIG. 7) or S. aureus (see FIG. 5 and FIG. 8) at either the one hour or four hour contact time point. Two of the three commercial solutions (ReNu and BioTrue) showed some effectiveness against S. marcescens at one hour of contact time, with all three solutions achieving adequate microbial reduction at four hours of contact time (see Table 2 and FIG. 9). Only one of the three commercial solutions (BioTrue) was effective against C. albicans at one hour of contact time, with all three requiring four hours of contact time to achieve some microbial reduction (see Table 2 and FIG. 10). All three commercial solutions showed some effectiveness against F. solani at one hour and four hour contact times (see Table 2 and FIG. 11). Two of the three commercial solutions (Opti-free and BioTrue) performed similarly to SOL01 and SOL02 at the four hour contact time against F. solani, indicating no growth of the fungi (see Table 2 and FIG. 10).

Both SOL01 and SOL02 were comprised primarily of stabilized chlorine dioxide. In addition to the chlorine-based disinfectant, a quaternary ammonium salt was included, which was shown to increase disinfection qualities Ammonium chloride was added to increase the available ammonium ions in conjunction with the quaternary ammonium salt. Peracetic acid was added to SOL02, which subsequently lowered the pH to 5.5, possibly accounting for the increase in cytotoxicity in this particular formulation. The cytotoxicity testing for SOL01 indicated a toxicity profile within an acceptable range.

The combination of the three active ingredients in SOL01 resulted in a completely sterilizing action against all of the challenge organisms with as little as one hour contact time. These results are in stark contrast to the performance of the three commercially-available MPDS, which showed little ability to disinfect against standard microbial organisms using contact times of either one hour or four hours. Performance of the commercial MPDS against the two strains of gram negative bacteria (P. aeruginosa and S. Marcescens) and the one strain of gram positive bacteria (S. aureus) was unexpected, as log reductions of these organisms have been achieved in past studies with the same MPDS. Disinfection qualities of the commercial MPDS against the selected fungi, C. albicans and F. solani, were more in line with expectations based upon previous work showing marginal or poor performance. Each of SOL01 and SOL02 in contrast was completely sterilizing against both of the fungal organisms after only one hour of contact time.

### Example 2

### Exemplary BBS Based Formulation with Biocidal Test Results

| Ingredient % (w/w) | A-2 | B-2 | C-2 | D-2 | E-2 | F-2 |
|---|---|---|---|---|---|---|
| Boric Acid | 0.850 | 0.850 | 0.850 | 0.850 | 0.850 | 0.850 |
| Sodium Borate | 0.130 | 0.130 | 0.130 | 0.130 | 0.130 | 0.130 |
| Sodium Chloride | 0.250 | 0.250 | 0.250 | 0.250 | 0.250 | 0.250 |
| Potassium Chloride | 0.250 | 0.250 | 0.250 | 0.250 | 0.250 | 0.250 |
| Tetronic 908 | 0.250 | 0.250 | 0.250 | 0.250 | 0.250 | 0.250 |
| Ammonium Chloride | — | — | — | 0.200 | 0.100 | 0.050 |
| Stabilized Chlorine | 0.04 | 0.02 | 0.01 | — | — | — |
| Dioxide* | | | | | | |
| pH | 7.39/7.44 | 7.39/7.44 | 7.39/7.44 | 7.39/7.44 | 7.39/7.44 | 7.39/7.44 |
| Osmolarity | 290 | 290 | 290 | 290 | 290 | 290 |

Biocidal Test Results

| | | | | | | |
|---|---|---|---|---|---|---|
| Staphylococcus | NG | NG | NG | Positive | Positive | Positive |
| aureus | | | | | | |
| Pseadomonas | NG | NG | NG | Positive | Positive | Positive |
| aeruginosa | | | | | | |
| Candida albicans | NG | NG | Positive | Positive | Positive | Positive |
| Fusarium solani | Positive | Positive | Positive | Positive | Positive | Positive |

*Stabilized chloride dioxide comprises about 3% sodium chlorite
NG: no growth
Positive: at least 1 colony forming unit (CFU) of growth.

### Example 3

### Exemplary Phosphate Buffer Based Formulation with Biocidal Test Results

| Ingredient % (w/w) | A-3 | B-3 | C-3 | D-3 | E-3 | F-3 |
|---|---|---|---|---|---|---|
| Sodium Phosphate, | 0.165 | 0.165 | 0.165 | 0.165 | 0.165 | 0.165 |
| Heptahydrate, | | | | | | |
| Dibasic | | | | | | |
| Sodium Phosphate, | 0.050 | 0.050 | 0.050 | 0.050 | 0.050 | 0.050 |
| Monohydrate, | | | | | | |
| monobasic | | | | | | |
| Sodium Chloride | 0.650 | 0.650 | 0.650 | 0.650 | 0.650 | 0.650 |
| Potassium Chloride | 0.250 | 0.250 | 0.250 | 0.250 | 0.250 | 0.250 |
| Tetronic 908 | 0.250 | 0.250 | 0.250 | 0.250 | 0.250 | 0.250 |
| Ammonium Chloride | — | — | — | 0.200 | 0.100 | 0.050 |
| Stabilized Chlorine | 0.04 | 0.02 | 0.01 | — | — | — |
| Dioxide* | | | | | | |
| pH | 7.0 | 7.0 | 7.0 | 7.0 | 7.0 | 7.0 |
| Osmolarity | 290 | 290 | 290 | 290 | 290 | 290 |

Biocidal Test Results

| | | | | | | |
|---|---|---|---|---|---|---|
| Staphylococcus | NG | NG | NG | Positive | Positive | Positive |
| aureus | | | | | | |
| Pseudomonas | NG | NG | NG | Positive | Positive | Positive |
| aeruginosa | | | | | | |
| Candida albicans | NG | NG | Positive | Positive | Positive | Positive |
| Fusarium solani | Positive | Positive | Positive | Positive | Positive | Positive |

*Stabilized chloride dioxide comprises about 3% sodium chlorite
NG: no growth
Positive: at least 1 colony forming unit (CFU) of growth.

### Example 4

### Exemplary Phosphate Buffer Based Formulation with Biocidal Test Results

| Ingredient % (w/w) | A-4 | B-4 | C-4 | D-4 | E-4 | F-4 |
|---|---|---|---|---|---|---|
| Sodium Phosphate, | 0.165 | 0.165 | 0.165 | 0.165 | 0.165 | 0.165 |
| Heptahydrate, | | | | | | |
| Dibasic | | | | | | |
| Sodium Phosphate, | 0.050 | 0.050 | 0.050 | 0.050 | 0.050 | 0.050 |
| Monohydrate, | | | | | | |
| monobasic | | | | | | |
| Sodium Chloride | 0.650 | 0.650 | 0.650 | 0.650 | 0.650 | 0.650 |
| Potassium Chloride | 0.250 | 0.250 | 0.250 | 0.250 | 0.250 | 0.250 |
| Tetronic 908 | 0.250 | 0.250 | 0.250 | 0.250 | 0.250 | 0.250 |
| Ammonium Chloride | — | 0.200 | 0.200 | — | — | — |
| Stabilized Chlorine | 0.01 | 0.01 | — | — | — | — |
| Dioxide* | | | | | | |
| C12-C14- | — | — | — | 0.050 | 0.025 | 0.0125 |
| alkyl(ethylbenzyl)dimethyl- | | | | | | |
| ammonium | | | | | | |
| chloride (80%) | | | | | | |
| pH | 7.0 | 7.0 | 7.0 | 7.0 | 7.0 | 7.0 |
| Osmolarity | 290 | 290 | 290 | 290 | 290 | 290 |

Biocidal Test Results

| | | | | | | |
|---|---|---|---|---|---|---|
| Staphylococcus | NG | NG | Positive | NG | NG | NG |
| aureus | | | | | | |
| Pseudomonas | NG | NG | Positive | NG | NG | NG |
| aeruginosa | | | | | | |
| Candida albicans | Positive | NG | Positive | NG | NG | NG |
| Fusarium solani | Positive | Positive | Positive | NG | NG | NG |

*Stabilized chloride dioxide comprises about 3% sodium chlorite
NG: no growth
Positive: at least 1 colony forming unit (CFU) of growth.

### Example 5

### Exemplary Phosphate Buffer Based Formulation with Biocidal Test Results

| Ingredient % (w/w) | A-5 | B-5 | C-5 | D-5 | E-5 | F-5 | G-5 |
|---|---|---|---|---|---|---|---|
| Sodium Phosphate, | 0.165 | 0.165 | 0.165 | 0.165 | 0.165 | 0.165 | 0.165 |
| Heptahydrate, | | | | | | | |
| Dibasic | | | | | | | |
| Sodium Phosphate, | 0.050 | 0.050 | 0.050 | 0.050 | 0.050 | 0.050 | 0.050 |
| Monohydrate, | | | | | | | |
| monobasic | | | | | | | |
| Sodium Chloride | 0.650 | 0.650 | 0.650 | 0.650 | 0.650 | 0.650 | 0.650 |
| Potassium Chloride | 0.250 | 0.250 | 0.250 | 0.250 | 0.250 | 0.250 | 0.250 |
| Tetronic 908 | 0.250 | 0.250 | 0.250 | 0.250 | 0.250 | 0.250 | 0.250 |
| Ammonium Chloride | — | — | 0.20 | — | 0.20 | 0.20 | 0.20 |
| Stabilized Chlorine | — | 0.01 | — | 0.01 | — | 0.01 | 0.01 |
| Dioxide* | | | | | | | |
| C12-C14- | 0.0125 | — | — | 0.0125 | 0.0125 | — | 0.125 |
| alkyl(ethylbenzyl)dimethyl- | | | | | | | |
| ammonium | | | | | | | |
| chloride (80%) | | | | | | | |
| pH | 7.0 | 7.0 | 7.0 | 7.0 | 7.0 | 7.0 | 7.0 |
| Osmolarity | 290 | 290 | 290 | 290 | 290 | 290 | 290 |

Biocidal Test Results

| | | | | | | | |
|---|---|---|---|---|---|---|---|
| Staphylococcus | NG | NG | Positive | NG | NG | Positive | NG |
| aureus | | | | | | | |
| Pseadomonas | Positive | Positive | Positive | NG | NG | NG | NG |
| aeruginosa | | | | | | | |
| Candida albicans | NG | Positive | Positive | NG | NG | Positive | NG |
| Fusarium solani | NG | Positive | Positive | NG | NG | Positive | NG |

*Stabilized chloride dioxide comprises about 3% sodium chlorite
NG: no growth
Positive: at least 1 colony forming unit (CFU) of growth.

### Example 6

### Exemplary Phosphate Buffer Based Formulation with Biocidal Test Results

| Ingredient % (w/w) | A-6 | B-6 | C-6 | D-6 | E-6 | F-6 | G-6 |
|---|---|---|---|---|---|---|---|
| Sodium Phosphate, | 0.165 | 0.165 | 0.165 | 0.165 | 0.165 | 0.165 | 0.165 |
| Heptahydrate, | | | | | | | |
| Dibasic | | | | | | | |
| Sodium Phosphate, | 0.050 | 0.050 | 0.050 | 0.050 | 0.050 | 0.050 | 0.050 |
| Monohydrate, | | | | | | | |
| monobasic | | | | | | | |
| Sodium Chloride | 0.650 | 0.650 | 0.650 | 0.650 | 0.650 | 0.650 | 0.650 |
| Potassium Chloride | 0.250 | 0.250 | 0.250 | 0.250 | 0.250 | 0.250 | 0.250 |
| Tetronic 908 | 0.250 | 0.250 | 0.250 | 0.250 | 0.250 | 0.250 | 0.250 |
| Ammonium Chloride | 0.20 | 0.20 | 0.20 | 0.20 | 0.20 | — | — |
| Stabilized Chlorine | 0.01 | 0.01 | 0.01 | 0.01 | 0.01 | — | — |
| Dioxide* | | | | | | | |
| C12-C14- | 0.0125 | 0.0075 | 0.0030 | 0.0010 | 0.0001 | 0.0075 | 0.0030 |
| alkyl(ethylbenzyl)dimethyl- | | | | | | | |
| ammonium | | | | | | | |
| chloride (80%) | | | | | | | |
| pH | 7.0 | 7.0 | 7.0 | 7.0 | 7.0 | 7.0 | 7.0 |
| Osmolarity | 290 | 290 | 290 | 290 | 290 | 290 | 290 |

Biocidal Test Results

| | | | | | | | |
|---|---|---|---|---|---|---|---|
| Staphylococcus | NG | NG | NG | NG | NG | NG | NG |
| aureus | | | | | | | |
| Pseudomonas | NG | NG | NG | NG | NG | NG | NG |
| aeruginosa | | | | | | | |
| Candida albicans | NG | NG | NG | NG | NG | NG | NG |
| Fusarium solani | NG | NG | NG | NG | Positive | NG | NG |

*Stabilized chloride dioxide comprises about 3% sodium chlorite
NG: no growth
Positive: at least 1 colony forming unit (CFU) of growth.

### Example 7

### Exemplary Phosphate/Borate Buffer Based Formulation with Biocidal Test Results

| Ingredient % (w/w) | A-7 | B-7 | C-7 | D-7 | E-7 | F-7 | G-7 | H-7 |
|---|---|---|---|---|---|---|---|---|
| Buffer | Borate | | | | Phosphate | | | |
| Stabilized Chlorine | 0.25 | | | 0.25 | 0.25 | | | 0.25 |
| Dioxide* | | | | | | | | |
| ammonium chloride | | 0.2 | | 0.2 | | 0.2 | | 0.2 |
| C12-14- | | | 0.001 | 0.001 | | | 0.001 | 0.001 |
| alkyl(ethylbenzyl) | | | | | | | | |
| dimethylammonium | | | | | | | | |
| chloride (80%) | | | | | | | | |

Biocidal Test Results

| | | | | | | | | |
|---|---|---|---|---|---|---|---|---|
| P. aeruginosa | NG | Positive | Positive | NG | NG | Positive | Positive | NG |
| S. aureus | NG | Positive | Positive | NG | NG | Positive | Positive | NG |
| C. albicans | Positive | Positive | NG | NG | Positive | Positive | NG | NG |
| F. solani | Positive | Positive | NG | NG | Positive | Positive | NG | NG |

*Stabilized chloride dioxide comprises about 3% sodium chlorite
NG: no growth
Positive: at least 1 colony forming unit (CFU) of growth.

Individual ingredient formulations (A-7 to C-7 and E-7 to G-7) and combinatory components formulation (D-7 and H-7) were tested for antimicrobial activity using standard procedural methodology (International Standards Organization). Minimum inhibitory concentrations (MICs) were defined as the lowest concentration of antimicrobial that will inhibit the visible growth of a micro-organism after incubation. MICs are used by diagnostic laboratories and as a research tool to determine the in vitro activity of new antimicrobials. In this particular set of MIC assays, either one or more of the active components of the novel formulation were added (100 μl) to an innoculum (10 ml) of a Gm+ (Staphylococcus aureus), Gm− (Pseudomonas aeruginosa), or fungi (Candida albicans, Fusarium albicans) each at an approximate concentration of 1.0E+04-1.0E+06 CFU/ml. After a 24 hour contact time, a sample of each test tube was plated on media specific to each bacteria or fungi, and growth was recorded. Growth of more than one colony on any of the agar plates was considered a failure (positive) of one or more of the components of the novel formulation to kill the microbial innoculum at that selected concentration of the component and/or mixture thereof. MIC testing usually were initiated with higher concentrations of the components and then halted once, by virtue of dilution, the MIC is achieved for each component and/or mixtures thereof.

In the present testing, three active components, stabilized chlorine dioxide, ammonium chloride, and C12-14-alkyl(ethylbenzyl) dimethylammonium chloride were provided either singularly or within a mixture in either a phosphate or borate buffering system. The buffering agents are considered inactive ingredients and provide pH adjustment and tonicity requirements for use in human health care applications and have no purported antimicrobial activity.

### Example 8

### Exemplary Formulations

### STR-325 Formulation

| | Concentration | |
|---|---|---|
| Ingredient | % (w/w) | Function |
| C12-C14 | 0.001 | Disinfectant |
| Alkyl(ethylbenzyl)dimethyl- | | |
| ammonium chloride (80%) | | |
| Stabilized chloride dioxide* | 0.01 | Disinfectant |
| Ammonium chloride | 0.20 | Disinfectant |
| Sodium phosphate monobasic | 0.012 | Buffer |
| (monohydrate) | | |
| Sodium phosphate dibasic | 0.195 | Buffer |
| (heptahydrate) | | |
| Sodium chloride | 0.10 | Tonicity agent |
| Potassium chloride | 0.20 | Tonicity agent |
| EDTA | 0.05 | Chelating agent |
| Tetronic ® 908 | 0.25 | Surfactant |
| Propylene glycol | 0.75 | Tonicity agent |
| Purified water | QS to 100 | Diluent |

*Stabilized chloride dioxide comprises about 3% sodium chlorite

### Formulation A Comprising a Phosphate Buffering System and:

| | Concentration | |
|---|---|---|
| Ingredient | % (w/w) | Function |
| C12-C14 | 0.001 | Disinfectant |
| Alkyl(ethylbenzyl)dimethyl- | | |
| ammonium chloride (80%) | | |
| Stabilized chloride dioxide* | 0.02 | Disinfectant |
| Ammonium chloride | 0.025 | Disinfectant |
| Tetronic ® 908 | 0.02 | Surfactant |
| Propylene glycol | 0.0075 | Tonicity agent |

*Stabilized chloride dioxide comprises about 3% sodium chlorite

### Formulation B Comprising a Phosphate Buffering System and:

| | Concentration | |
|---|---|---|
| Ingredient | % (w/w) | Function |
| C12-C14 | 0.003 | Disinfectant |
| Alkyl(ethylbenzyl)dimethyl- | | |
| ammonium chloride (80%) | | |
| Stabilized chloride dioxide* | 0.03 | Disinfectant |
| Ammonium chloride | 0.025 | Disinfectant |
| Tetronic ® 908 | 0.02 | Surfactant |
| Propylene glycol | 0.0075 | Tonicity agent |

*Stabilized chloride dioxide comprises about 3% sodium chlorite

Microbial data summary for Formulation A:

| Target Pathogen | Organism | Contact time | | Maximum killing attained |
|---|---|---|---|---|
| Protozoa | Acanthamoeba | 30 | min | ≥9.9 × 10⁴ |
| | castellanii | | | |
| | Acanthamoeba | 30 | min | ≥9.9 × 10⁴ |
| | polyphaga | | | |
| Bacteria | S. Aureus | 1 | min | ≥9.9 × 10⁷ |
| | MRSA | 1 | min | Direct kill |
| | P. aeruginosa | 10 | min | ≥9.9 × 10⁷ |
| | Serratia marcenses | 10 | min | ≥9.9 × 10⁷ |
| | MDR-Acinetobacter | 1 | min | Direct kill |
| | baumannii | | | |
| Virus | HSV-1 | 60 | min | ≥9.9 × 10⁴ |
| | HSV-2 | 60 | min | ≥9.9 × 10³ |
| | HuAdV-3 | 60 | min | ≥9.9 × 10³ |
| | HuAdV-4 | 60 | min | ≥9.9 × 10² |
| | HuAdV-5 | 60 | min | ≥9.9 × 10² |
| | HIV-2F | 30 | min | ≥9.9 × 10² |
| Fungus | Fusarium solani | 1 | min | >6 log reduction |
| | Candida Albicans | 1 | min | >6 log reduction |

### Example 9

### Formulation is Effective in Killing Infectious Protozoa Acanthamoeba spp

Free living amebae of the genus Acanthamoeba are saprophytic protozoa that are ubiquitous in the environment. Particular species of the genus, including Acanthamoeba castellanii, can cause severe infections in man. One manifestation of A. castellanii infection includes extremely rare opportunistic granulatomas encephalitis that can develop only after accidental oral/nasal insufflation. A more common, albeit rare, disease syndrome includes Acanthamoeba keratitis resulting from inadvertent ocular exposure to environmental sources. The latter condition is considered a severe form of keratitis that can lead to long term sequelae including blindness if left untreated. A significant increase of diagnosed cases of Acanthamoeba keratitis, particularly among contact lens users, has been observed over the last decade.

The anti-protozoal efficacy of STR-325 (comprising stabilized chlorine dioxide, ammonium chloride, and C12-14- alkyl(ethylbenzyl) dimethylammonium chloride as shown in example 8) against A. castellanii trophozoites in an experimental colorimetric assay (McBride, J, Ingram, P R, Henriquez, F L, Roberts, C W. J Clin Microb, Feb;43(2):629-34, 2005) was tested. The efficacy against two leading over-the-counter multipurpose solutions (MPS), chlorhexidine (5% solution), and povidone-iodine (10% solution) was evaluated.

### Acanthamoeba Castellani Acquisition and Propagation

The assay in this study used cultured A. castellanii trophozoites originating from corneal scrapings and was obtained from ATCC (Manassas, Va.). Trophozoites were grown at 25° C. in Yeast Extract Peptone Dextrose (YPD) media with 1% Penicillin/Streptomycin/Amphotericin B in separate co-cultures.

### Trophozoites

Trophozoites were enumerated using a Coulter cell counter and verified by light microscopy and a hemocytometer. Alamar blue was used as a vitality dye, and effectively measures cellular respiration. The intensity of the dye degraded at an equivalent rate of remaining active trophozoites in solution; thus the reciprocal of the measured absorbance of the dye was correlative to the remaining active trophozoites in the test well. Predetermined concentrations of active trophozoites (1.2E+3 cells/well) were used for the assay, and exactly 25 μl of either STR-325, dilutions thereof, or comparator MPS were aliquoted into A. castellanii-loaded wells; testing was performed in sextuplicate (6 wells/solution). Disinfectant-A. castellanii solutions were incubated and then read on a spectrophometer at 570 nm.

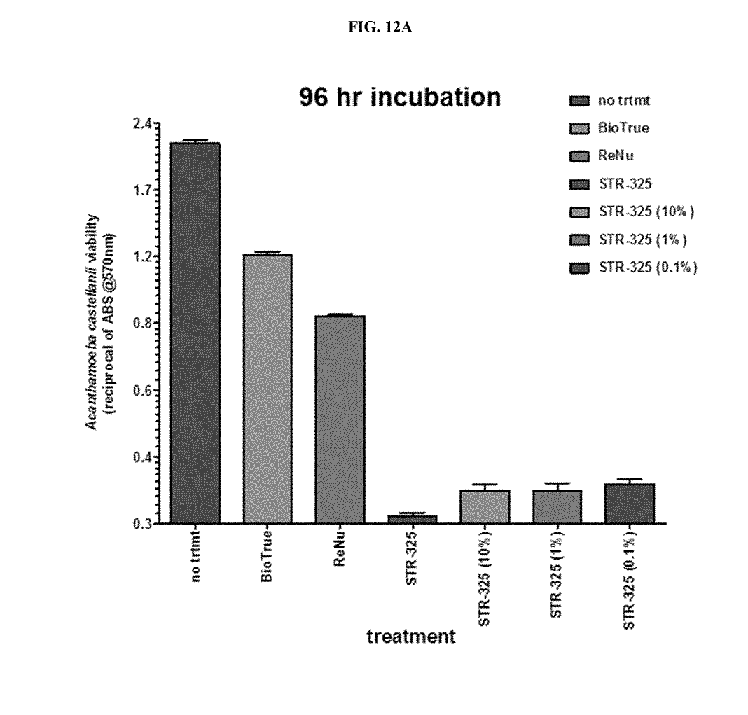

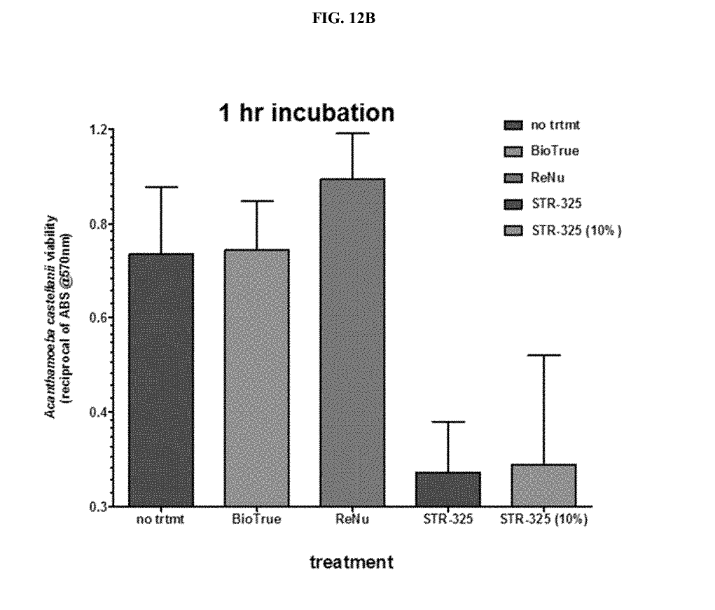

Results of the testing comparing STR-325 with comparator solutions (ReNu, Biotrue) are shown in FIG. 12A and FIG. 12B (96 h or 1 h incubation). STR-325 was efficacious in killing all trophozoites at both incubation times, and maintained effectiveness even when serially diluted (1:10). Both comparator solutions (ReNu, Biotrue) had marginal effects on reduction of active trophozoites at 96 hr incubation, and no effect at the shorter (1 hr) incubation time.

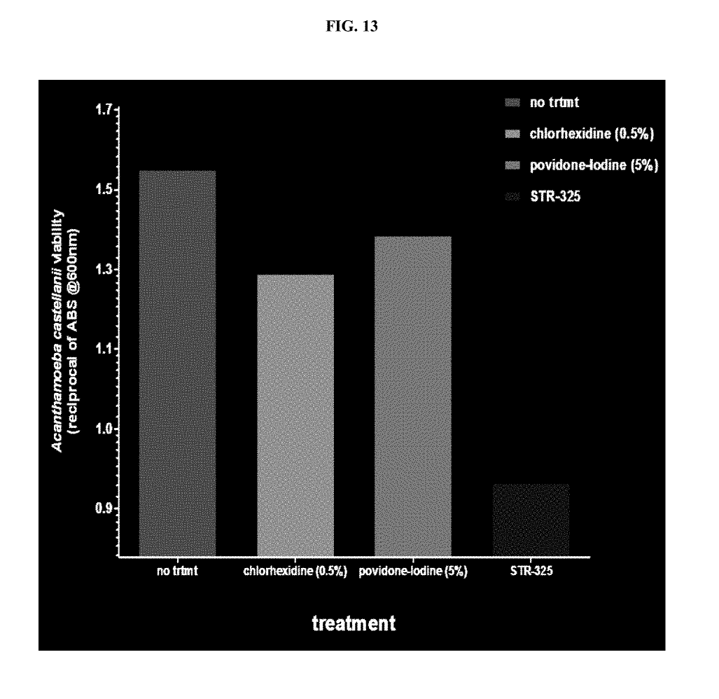

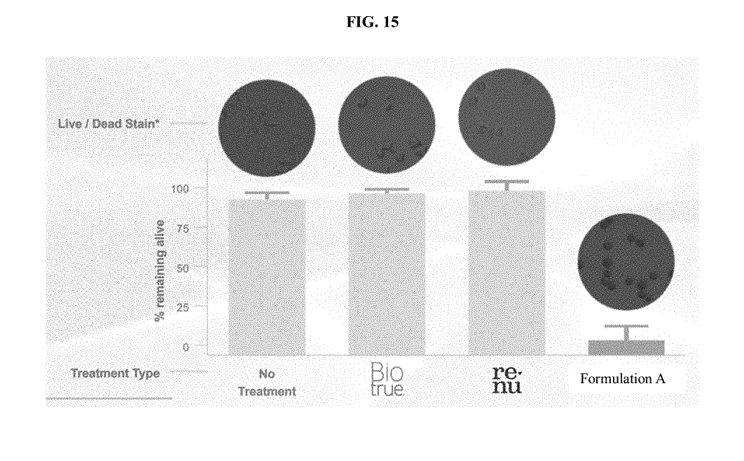

Results of the testing comparing STR-325, chlorhexidine, and povidone-iodine are shown in FIG. 13. FIG. 15 shows elimination of A. castellanii after 30 minute exposure with Formulation A.

### Cysts

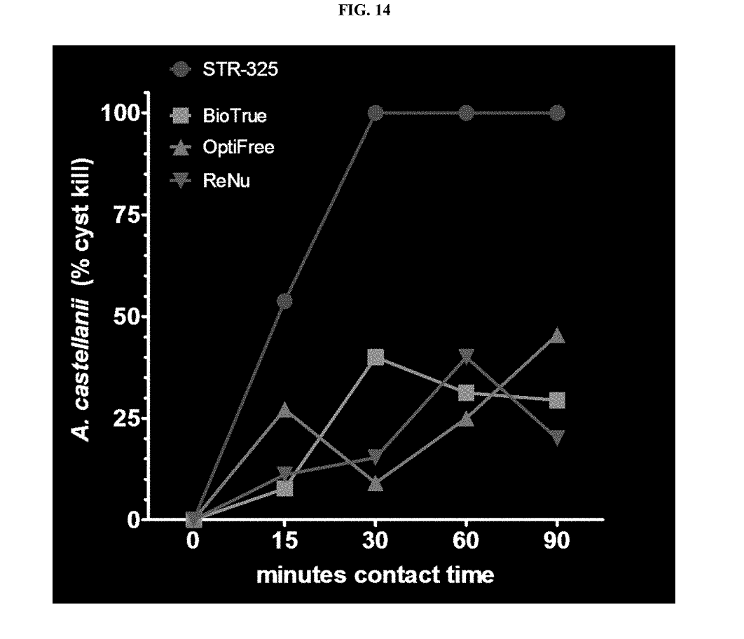

Activity against the cyst form was determined via a tryptan blue assay and compared to currently marketed contact lens cleaners. Cysts were induced via 72 h incubation of 100 μL 10⁶ cells/mL in 10 mL encystment media (95mM NaCl, 5 mM KCl, 8 mM MgSO₄, 0.4 mM CaCl₂, 1 mM NaHCO₃, 20 mM Tris-HCl; pH 9.0). Cysts were washed twice via centrifugation for 5 min at 1000 rpm, and diluted with PBS to 10⁵ cells/mL. Cysts and test solutions were incubated at room temperature in 1:1 ratio (10 μL:10 μL) for duration of contact time (15-90 min). 10 μL tryptan blue was used to visualize microscopically using a hemocytometer with a count of at least 9 fields within the grid. FIG. 14 shows the results. Of the comparator contact lens solutions tested, a maximum cyst kill of about 20-30% was achieved after 4 h contact time. STR-325 achieved 100% kill after only 30 min contact time.

### Example 10

### Exemplary Formulation and Compounding Procedure

The compounding tank was charged with USP water (80% total final volume). The following ingredients were then added: sodium phosphate monobasic, monohydrate; sodium phosphate dibasic, heptahydrate; sodium chloride; and potassium chloride. The mixture was stirred for NLT (not less than) 15 minutes. Tetronic® 908 was slowly added into the compounding tank and mixed until dissolved completed (NLT 30 minutes). The mixture was cooled below 45° C. and the following ingredients were added: ammonium chloride, stabilized chlorine dioxide, and C12-C14-alkyl(ethylbenzyl)dimethylammonium chloride. The resulting mixture was mixed for NLT 30 minutes. The pH and osmolarity were measured (Targeted pH=7.0, and Osmolarity 300). Sterility: use 0.22 μm sterilizing filter (Minipore).

### Example 11

### HIV Antiviral Testing

The ophthalmic composition STR-325 was tested for antiviral activity against Human Immunodeficiency Virus (HIV) as HIV-2f. A 6.6 μl aliquot of media containing 600 TCID₅₀ of HIV-2f was added to 993.4 μl of the STR-325. The co-mixed virus- STR-325 was gently vortexed, and allowed to incubate for either 5, 30, or 60 minutes contact time at 37° C. Phosphate buffered saline (PBS) was used as a control treatment in separate tubes containing virus Immediately following incubation, the test and control mixtures were applied to either CEMx174 or MT-4 cell suspensions and allowed to incubate with the cells in a 6-well plate format for 96 hours. Thereafter, the cytopathogenic effects (CPE) were observed in each well on each plate for qualitative analysis of the formation of viral syncytia and other signs of CPE as a result of viral infection. Results showed that minimal syncytia and minimal dead cells were present in the wells treated with STR-325, and wells looked similar to uninfected, normal suspension-growth cells. In contrast, the infected, PBS-control treated wells contained syncytia and dead cells similar to the infected controls. Thereafter, sample-containing media was harvested from each well on the first day that CPE is observed in the cells. These media samples were tested for the presence and concentration of SIV p27 antigen using ELISA technology in a commercial prepared kit (Zeptometrix Inc.). The presence and concentration of p27 antigen is a product of, and directly correlated to, viral infection and active replication and can be used as a quantitative measurement of viral activity or lack thereof. The results of the p27 viral antigen testing (FIG. 2) shows nearly complete viral inhibition in the samples containing virus+STR-325 in contrast to the infected, untreated samples. The levels of p27 antigen in the virus+STR-325 samples are similar in concentration to the uninfected, sentinel samples. Results are shown in table 3.

TABLE 3

| | | PBS | | |
|---|---|---|---|---|
| | | control | PBS | STR-325 |
| | Incubation | (Uninfected | control | (1:10) + |
| Cell line | time (min) | cells) | (HIV2f) | HIV2f |
| CEMx174 | 5 | 0.06 | 2.80 | 0.19 |
| | 30 | 0.07 | 2.73 | 0.09 |
| | 60 | 0.06 | 2.46 | 0.08 |
| MT-4 | 5 | 0.07 | 0.39 | 0.09 |
| | 30 | 0.06 | 0.40 | 0.08 |
| | 60 | 0.06 | 0.42 | 0.08 |

### Example 12

### Rabbit Eye Irritation Studies

All commercially acquired rabbits (New Zealand White, 2.0-2.9 Kg) were examined prior to testing and were found to be healthy with no previous study inclusion (naïve animals) from ophthalmic-associated testing. A total of 18 rabbits were used in these studies. Rabbits were each administered into the right eye via eyedropper instillation approximately 10 ml of the STR-325 ophthalmic solution, three times/day during a nine-hour day. Animals were observed after each instillation and eye condition and irritation was graded according to standard categorical, cumulative scoring scale, commonly referred to as the ‘Draize test’ as described. No physiological reaction to the solution instillation was observed in any animals receiving the instillation. Upon scoring of the eye, no measurable irritation indicative of the inflammatory response was observed in any animal receiving the solution. No eye scored higher than a ‘2’ in the Draize scoring regiment, which is indicative of no irritation. Other indicators of damage or inflammation, including corneal staining, was not observed after instillation into the eye of any of the study subjects. The pH of the solution used was 7.1, which matches eye physiological pH. All animals in this trial were euthanized after termination the study, and pathology performed on the eyes of the animals treated with the solution. The results of the pathological analysis of tissues showed no abnormalities as reported by the veterinary pathologist.

### Example 13

### Rabbit Model of Adenoviral Keratoconjunctivitis

### Rabbits, Virus, and Infection

Corneas of 20 NZW rabbits (6-8 lb) are scarified with a 3×3 cross-hatch pattern scratch (topical anesthesia of proparacaine hydrochloride ophthalmic solution USP 0.5% is administered; Bausch & Lomb Inc., Tampa, Fla.), and then the corneas are inoculated with the Ad5 strain at 2×106 PFU/eye. The animals are then randomly assigned to one of two treatment groups (ten rabbits/group): STR-325 and balanced salt solution (BSS; Alcon Laboratories).

A slit lamp examination is performed daily before drug treatment. Treatments consisted of 50-μL drops/eye. The drug solutions for the topical applications are stored in brown sterile glass bottles and separately dispensed with disposable pipettes.

### Infectious Virus Titers

Tears are collected daily from the eyes under the eyelids and nictitating membranes of each rabbit with a cotton swab, starting 3 hours after infection; subsequently, the swabs are taken daily in the early morning before administration of drugs over a 10-day period. Drug treatment began 12 hours after viral inoculation and continued for 24, 48, 72, 96, 120, 144, and 168 hours. Virus is eluted from the swabs with RPMI 1640 medium (1 mL; 1×, without 1-glutamine; Cellgro; Mediatech Inc., Manassas, Va.) and kept at −80° C. overnight or until ready for use. Volumes of 200 μL of 10-fold serial dilutions of original eluate/swab are plated in triplicate to individual wells of a 24-well plate containing subconfluent A549 cells and allowed to incubate for 1 hour. Subsequently, excess media are decanted and an overlay of RPMI 1640 containing 0.5% methylcellulose is added to each well. Plates are incubated at 37° C. and 5% CO2 for 3 days, after which monolayers are fixed with 10% formaldehyde, washed, and stained with crystal violet (Hucker's modified solution diluted 1:10 with 20% ethanol and 80% H₂O), and the plaques are counted.

### Slit Lamp Examination and Scoring of Six Clinical Parameters

After corneal Ad5 infection, the eyes are monitored daily in the early morning by two independent observers with a slit lamp microscope (Eye Cap; Haag-Streit International, Mason, OHIO) before administration of the drugs in a masked fashion. The severity of adenoviral keratoconjunctivitis is assessed by scoring six clinical parameters: scleral inflammation (injection), ocular neovascularization, eyelid inflammation (redness), friability of vasculature, inflammatory discharge (pus), and epiphora (excessive tearing). The clinical scoring is based on a weighted scale, using the scoring system described in the table 4.

TABLE 4

| | Clinical scores | | | |
|---|---|---|---|---|
| | 0 | | | |
| | (symptomatic of | | | |
| | naïve uninfected | +1 | +2 | +3 |
| Ocular | with no | (slight | (moderate | (severe |
| parameters | manifestations) | manifestations) | manifestations) | manifestations) |
| Scleral | No redness, no | Reddish tinge, | Red, prominent | Hyperemia with |
| inflammation | prominent blood | blood vessels more | blood vessel net- | individual blood |
| | vessels and no | visible and local- | work and localized | vessels indistin- |
| | chemosis | ized chemosis | chemosis | guishable and |
| | | | | chemosis |
| Ocular | No visible blood | Developing blood | Blood vessel net- | Prominent blood |
| neovascularization | vessels | vessel network | work with 26-75% | vessel network |
| | | with <25% of the | of the surface af- | with >75% of the |
| | | surface affected | fected | surface vascularized |
| Friability of | Nictitating mem- | Nictitating mem- | Nictitating mem- | Nictitating mem- |
| vasculature* | brane and inside of | brane and inside of | brane and inside of | brane and inside of |
| | lower eyelid do | lower eyelid in- | lower eyelid show | lower eyelid show |
| | not show any sign | flamed and reddish | sparse flecks of | ooze of reddish |
| | of bleeding and no | tinge on cotton | blood and red col- | watery fluid and |
| | coloration on cot- | surface after | oration on ~50% | red coloration >50% |
| | ton surface after | swabbing | of cotton surface | of cotton |
| | swabbing | | after swabbing | surface after |
| | | | | swabbing |
| Inflammatory | No mucous dis- | Mucous discharge | Mucous discharge | Mucous discharge |
| discharge | charge | collecting at the | collecting at the | collecting at the |
| | | caruncle and lac- | caruncle, lacrimal | adnexa and caus- |
| | | rimal apparatus | apparatus and eye- | ing peri-ocular fur |
| | | | lid margin | to be matted down |
| | | | | (pseudomembranes) |
| Eyelid | Eyelid margins | Eyelid margins | Eyelid margins | Eyelid margins |
| inflammation | thin with no | showing localized | showing localized | swollen, red and |
| | visible redness | swelling and | swelling and visi- | engorged outer |
| | | redness | ble blood vessel | parts of eyelids |
| | | | network | |
| Epiphora | Moist corneal | Watery fluid col- | Watery fluid col- | Watery fluid col- |
| | surface | lection mainly at | lection mainly at | lection mainly at |
| | | the lower eyelid | the lower eyelid | the lower eyelid |
| | | margin | margin and wetness | margin, wetness of |
| | | | of the entire | the entire outer |
| | | | outer part of | part of eyelids and |
| | | | eyelids | pseudomembranes |

Clinical scores are recorded at 3, 24, 48, 72, 96, 120, 144, 168, 192, and 240 hours after infection. The mean±SEM clinical score is calculated from the daily cumulative of the six clinical parameters as independent events, to evaluate improvement of the rabbit eyes. In addition, the cumulative score values of the six clinical parameters in each drug treatment group are weighted vertically to provide a method of comparison.

### Example 14

### Phase 1 Clinical Trial of STR-325 in Acanthamoeba Keratitis

This study is a randomized, double-masked, placebo-controlled, multiple center, parallel-group Phase 1 study to evaluate the safety and tolerability of STR-325 ophthalmic solution compared to placebo in healthy subjects. The primary objective of the study is to establish the ocular safety, tolerability, and systemic safety of STR-325 in healthy subjects. Safety and tolerability will be compared to those of a placebo.

### Study Design:

Allocation: Randomized
Intervention Model: Parallel Assignment
Masking: Double Blind (Subject, Caregiver, Investigator, Outcomes Assessor)
Primary Purpose: Treatment

### Primary Outcome Measures:

Number of Subjects With Dose-limiting Adverse Events [Time Frame: up to 21 days from date of randomization]

### Other Outcome Measures:

Systolic Blood Pressure [Time Frame: Baseline and Day 14]
Change from baseline systolic blood pressure
Visual Acuity [Time Frame: Baseline and Day 14]: Change from baseline Visual acuity. Best corrected visual acuity was reported in decimal fraction. A decrease of visual acuity during treatment is considered a negative safety outcome.
Ocular Surface Disease Index-OSDI [Time Frame: Baseline and Day 14]: Change from baseline OSDI at D 14. An increase of OSDI values with treatment represents a negative outcome.
Conjunctival Examination [Time Frame: Baseline and Day 14]: change from baseline conjunctival staining at Day 14 Staining with lissamine green is used. The density of staining was graded with the Oxford Score. An increase in the score after treatment represents a negative outcome

| Arms | Assigned Interventions |
|---|---|
| Experimental: STR-325 eye drops | Drug: STR-325 |
| Placebo Comparator: Vehicle | Drug: Vehicle |

### Eligibility

Ages Eligible for Study: 18 Years to 55 Years (Adult)
Sexes Eligible for Study: All
Accepts Healthy Volunteers: Yes

### Criteria

### Inclusion Criteria:

able and willing to give informed consent.
man or woman of any race and 18 to 55 years of age, inclusive.
Body Mass Index of 20-30 kg/m2
willing and able to attend required study visits.
bilateral visual acuity >6/10.
intraocular pressure (TOP) of 14-21 mmHg
ophthalmologic examination without abnormalities.
medical history without major pathology.
laboratory test results without deviations from the normal range.
female subjects of childbearing potential with negative urine pregnancy test and using effective contraception during the study.

### Exclusion Criteria:

presence of bacterial ocular infections.
presence of any concomitant ocular pathology.
performing activities likely to result in an irritated conjunctiva during the study (including heavy alcohol intake, swimming in chlorinated water and heavy smoking).
contact lenses wearing.
ocular surface fluorescein staining score >3.
use of topical or systemic antibiotics, antihistamines, decongestants and non-steroidal anti-inflammatory agents as well as steroids within 7 days before screening.
known or suspected allergy to ingredient of the test treatments.
ocular surgery performed within 12 months before screening.
participation in another clinical study in the preceding 30 days.
one functional eye.
pregnancy or breastfeeding.
use of recreational drugs.

What is claimed is:

1. A method of treating or preventing an eye condition in a subject in need thereof, the method comprising administering to the subject in need thereof a pharmaceutical composition comprising:
(a) a chlorite salt;
(b) a quaternary ammonium salt; and
(c) ammonium chloride.

2. The method of claim 1, wherein the eye condition is an inflammation.

3. The method of claim 2, wherein the inflammation is conjunctivitis, keratitis, blepharitis, meibomianitis, endophthalmitis, or any combination thereof.

4. The method of claim 2, wherein the inflammation is conjunctivitis.

5. The method of claim 2, wherein the inflammation is acanthamoebic keratitis.

6. The method of claim 2, wherein the inflammation is caused by a bacterial infection.

7. The method of claim 2, wherein the inflammation is caused by a viral infection.

8. The method of claim 2, wherein the inflammation is caused by a fungal infection.

9. The method of claim 1, wherein the chlorite salt is present in an amount ranging from about 0.0001% to about 1% (w/w).

10. The method of claim 1, wherein the chlorite salt is an alkali metal chlorite salt.

11. The method of claim 10, wherein the alkali metal chlorite salt is sodium chlorite.

12. The method of claim 11, wherein the sodium chlorite is provided as a stabilized chlorine dioxide solution.

13. The composition of claim 12, wherein the stabilized chlorine dioxide is present in an amount ranging from about 0.005% to about 1% (w/w).

14. The method of claim 1, wherein the quaternary ammonium salt is present in an amount ranging from about 0.0001% to about 0.5% (w/w).

15. The method of claim 1, wherein the quaternary ammonium salt comprises C12 or C14 alkyl chain.

16. The method of claim 1, wherein the quaternary ammonium salt is not benzalkonium chloride.

17. The method of claim 1, wherein the quaternary ammonium salt is a polymeric quaternary ammonium salt.

18. The method of claim 1, wherein the quaternary ammonium salt is C12-C14-alkyl(ethylbenzyl)dimethylammonium chloride.

19. The method of claim 1, wherein the ammonium chloride is present in an amount ranging from about 0.001% to about 2% (w/w).

20. The method of claim 1, wherein the pharmaceutical composition further comprises a pharmaceutically acceptable excipient.

21. The method of claim 20, wherein the pharmaceutically acceptable excipient is selected from a surfactant, a chelating agent, a tonicity agent, a viscosity agent, a non-aqueous solvent, a buffer, a pH adjusting agent, an antioxidant, and any combinations thereof.

22. The method claim 1, wherein the pharmaceutical composition further comprises a buffer to maintain the pH between about 7 and about 8.

23. The method of claim 22, wherein the pH is about 7.4.

24. The method of claim 1, wherein the pharmaceutical composition is in the form of a solution, an emulsion, a solid dispersion, a colloidal dispersion, a suspension, a gel, an ointment, a cream, a lyophilized powder, a nanoparticle formulation, or any combinations thereof.

25. The method of claim 1, wherein the pharmaceutical composition is applied with a pad, cloth, wipe, sponge, brush, or cotton tipped applicator.

26. The method of claim 1, wherein the pharmaceutical composition is in the form of a surgical wash or irrigation solution.

27. The method of claim 26, wherein the surgical wash or irrigation solution is applied to the eye of the subject in need thereof.

28. The method of claim 27, wherein the surgical wash or irrigation solution is applied to the eye of the subject in need thereof prior to a surgery.

29. The method of claim 27, wherein the surgical wash or irrigation solution is applied to the eye of the subject in need thereof after a surgery.

30. The method of claim 1, wherein the pharmaceutical composition is applied by topical administration.
